(19) United States
(12) Patent Application Publication
(10) Pub. No.: US 2022/0395587 A1
(43) Pub. Date: Dec. 15, 2022

(54) CELLULAR MOLECULAR THERANOSTICS NANOPROBE SYSTEMS AND METHODS

(71) Applicant: Duke University, Durham, NC (US)

(72) Inventors: Tuan VO-DINH, Chapel Hill, NC (US); Hsin-Neng WANG, Durham, NC (US); Bridget M. CRAWFORD, Durham, NC (US)

(21) Appl. No.: 17/776,663

(22) PCT Filed: Nov. 16, 2020
(86) PCT No.: PCT/US20/60644

§ 371 Date: May 13, 2022

Related U.S. Application Data
(60) Provisional application No. 62/935,883, filed on Nov. 15, 2019.

(51) Int. Cl. A61K 49/00; C12Q 1/6883; C12Q 1/6895; A61K 47/69

(52) U.S. Cl. CPC A61K 49/00; C12Q 1/6883; C12Q 1/6895; A61K 47/6929; A61K 47/6923; C12Q 1/6825

(57) ABSTRACT

A nanoprobe system for in vivo use comprises a plasmonic-active nanoparticle and a molecular probe system. The molecular probe system comprises an oligonucleotide capable of forming a stem-loop configuration, having a first end and a second end, wherein the oligonucleotide is immobilized to the plasmonic-active nanoparticle at the first end and labeled with a Raman reporter at the second end, a placeholder strand at least partially bound to the oligonucleotide, and an attachment mechanism for attachment to a cell membrane.

### CROSS-REFERENCE TO RELATED APPLICATIONS

This application is a U.S. National Stage Application of International Patent Application No. PCT/US20/60644, filed on Nov. 16, 2020, which claims priority to U.S. Provisional Patent Application No. 62/935,883, filed on Nov. 15, 2019, which are both incorporated by reference herein in their entirety.

### BACKGROUND

There is an important need to develop practical, efficient, “label-free” diagnostic and seamless therapeutic methods and systems to monitor the viability of cellular systems and, in some instances, induce gene regulation, for use in a variety of biomedical applications (e.g., cancer diagnostics and therapy, infectious disease monitoring and treatment, regeneration of damaged tissue, stem cell therapy, tissue engineering, use of artificial organs, treatment of disease, etc.). For example, methods and systems to detect early molecular biomarkers (miRNAs, mRNA, DNA, proteins, peptides, metabolites, etc.) of cellular systems and treat associated diseases are needed.

### SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In a first aspect of the invention, a nanoprobe system for in vivo use comprises a plasmonic-active nanoparticle and a molecular probe system. The molecular probe system comprises an oligonucleotide capable of forming a stem-loop configuration, having a first end and a second end, wherein the oligonucleotide is immobilized to the plasmonic-active nanoparticle at the first end and labeled with a Raman reporter at the second end, a placeholder strand at least partially bound to the oligonucleotide, and an attachment mechanism for attachment to a cell membrane.

In a second aspect of the invention, a method of in vivo monitoring and/or detection comprises administering a nanoprobe system to a subject, the nanoprobe system comprising a plasmonic-active nanoparticle; an oligonucleotide having a first end and a second end, wherein the oligonucleotide is immobilized to the plasmonic-active nanoparticle at the first end and labeled with a Raman reporter at the second end; a placeholder strand complimentary to and at least partially bound to the oligonucleotide; and an attachment mechanism for attachment of the nanoprobe system to a cell membrane. The placeholder strand targets a specific sequence and upon exposure of the nanoprobe system to the specific target sequence, the placeholder strand leaves the oligonucleotide in favor of the target sequence, allowing the oligonucleotide to fold into a closed stem-loop configuration whereby the Raman reporter is near or on the plasmonic-active nanoparticle surface thereby yielding a SERS signal. The SERS signal is detected with a detection device.

In a third aspect of the invention, a method of in vivo therapy comprises administering a nanoprobe system to a subject in need of a desired therapy, the nanoprobe system comprising a plasmonic-active nanoparticle; an oligonucleotide having a first end and a second end, wherein the oligonucleotide is immobilized to the plasmonic-active nanoparticle at the first end and labeled with a Raman reporter at the second end; a placeholder strand complimentary to and at least partially bound to the oligonucleotide; a component for inducing molecular regulation linked to the placeholder strand; and an attachment mechanism for attachment of the nanoprobe system to a cell membrane. The placeholder strand targets a specific sequence, and upon exposure of the nanoprobe system to the specific target sequence, the placeholder strand leaves the oligonucleotide in favor of the target sequence, allowing the oligonucleotide to fold into a closed stem-loop configuration whereby the Raman reporter is near or on the plasmonic-active nanoparticle surface thereby yielding a SERS signal. The component for inducing molecular regulation separates from the placeholder strand thereby initiating the desired therapy. The SERS signal is detected with a detection device.

### BRIEF DESCRIPTION OF THE DRAWINGS

The accompanying Figures are provided by way of illustration and not by way of limitation. The foregoing aspects and other features of the disclosure are explained in the following description, taken in connection with the accompanying example figures relating to one or more embodiments.

FIG. 1 is a schematic diagram of intracellular and extracellular nanoprobe systems attached to a membrane system for in vivo implantation.

FIG. 2 is a schematic diagram of exemplary nanoprobes for intracellular sensing.

FIG. 3 is a schematic diagram of exemplary nanoprobes for extracellular sensing.

FIG. 4 is a schematic diagram of an exemplary membrane-linked nanoprobe for extracellular sensing.

FIG. 5 is a schematic diagram of an exemplary membrane-linked nanoprobe for both intracellular and extracellular sensing.

FIG. 6 is a schematic diagram of iMS Nanoprobes for Intracellular Sensing.

FIG. 7 is a schematic diagram of membrane-linked iMS nanoprobes for extracellular sensing.

FIG. 8 is a schematic diagram of membrane-linked iMS nanoprobes for intracellular sensing.

FIG. 9 is a schematic diagram of membrane-linked iMS nanoprobes via receptors (antibody, cell surface receptor, etc.) for extracellular sensing.

FIG. 10 is a schematic diagram of membrane-linked iMS nanoprobes for Intracellular and Extracellular Sensing.

FIG. 11 is a schematic diagram showing the operating principle of a “Molecular Sentinel” (MS) nanoprobe.

FIG. 12 is a schematic diagram illustrating an embodiment of an iMS nanoprobe.

FIGS. 13A & 13B are schematic diagraming showing an exemplary Ims using a gold nanostar as the nanoparticle platform. FIGS. 14A & 14B are schematic diagrams showing an exemplary nanoprobe system including a linker.

FIGS. 15A, 15B & 15C are schematic diagrams showing an exemplary iMS nanoprobe system using temperature cycling.

FIG. 16 is a schematic diagram showing integration of an iMS nanoprobe system with an aptamer.

FIGS. 17A & 17B are schematic representations of using multiple nanoprobe systems for multiplex detection and blocking (capture) of mRNA and lncRNA.

FIGS. 18A & 18B are schematic representations of using multiple nanoprobe systems for additive detection and blocking (capture) of mRNA and lncRNA.

FIG. 19 is a schematic diagram depicting the molecular process of siRNA.

FIGS. 20A I-VI are schematic diagrams showing a Direct Theranostics Scheme using an iMS nanoprobe system and ds-siRNA.

FIG. 20B I-VI are schematic diagrams showing a different exemplary embodiment of a Direct Theranostics Scheme using an iMS nanoprobe system and ds-siRNA.

FIGS. 21A I-VI are schematic diagrams showing a Direct Theranostics Scheme using an iMS nanoprobe system and ss-siRNA.

FIG. 21B I-VI are schematic diagrams showing a different exemplary embodiment of a Direct Theranostics Scheme using an iMS nanoprobe system and ss-siRNA.

FIGS. 22A-F are schematic diagrams of a Theranostics system using Photocleavage as a stimulus with iMS and ds-siRNA.

FIGS. 23A-E are schematic diagrams of a Theranostics system with iMS and ds-siRNA, with no photocleavage.

FIGS. 24A-F are schematic diagrams showing a different exemplary embodiment of a Theranostics system using Photocleavage with iMS and ds-siRNA.

FIGS. 25A-E are schematic diagrams of a different exemplary embodiment of a Theranostics system with iMS and ds-siRNA.

FIGS. 26A-E are schematic diagrams showing an alternative exemplary embodiment of a Theranostics system Using Photocleavage with iMS and ss-siRNA.

FIGS. 27A-D are schematic diagrams of an alternative exemplary embodiment a Theranostics system with iMS and ss-siRNA.

FIGS. 28A-F are schematic diagrams of an alternative exemplary embodiment a MS-siRNA Theranostics system with ds-siRNA and Alternative linker Using Photocleavage.

FIGS. 29A-E are schematic diagrams of an alternative exemplary embodiment a iMS-siRNA Theranostics system with ss-siRNA Using Photocleavage.

FIGS. 30A-C are schematic diagrams of an alternative exemplary embodiment a Theranostics system Using Photothermal Heating with one laser.

FIGS. 31A-C are schematic diagrams of an alternative exemplary embodiment a Theranostics system using Alternative Photothermal Heating with two different lasers.

FIGS. 32A-C are schematic diagrams of an alternative exemplary embodiment a MS-siRNA Theranostics system Using Thermal Heating.

FIGS. 33A-B are schematic diagrams of an exemplary embodiment of a Theranostics system comprising both siRNA and an aptamer.

FIGS. 34A-C provide another exemplary schematic of a nanoprobe system having an iMS-aptamer.

FIGS. 35A-F are additional exemplary schematics of a nanoprobe system.

FIGS. 36A-J are schematic diagrams showing exemplary embodiments of plasmonics-active nanostructures: (A) Metal nanoparticle, (B) Dielectric nanoparticle core covered with metal nanocap, (C) Spherical metal nanoshell covering dielectric spheroid core, (D) Oblate metal nanoshell covering dielectric spheroid core, (E) Metal nanoparticle core covered with dielectric nanoshell, (F) Metal nanoshell with protective coating layer, (G) Multi-layer metal nanoshells covering dielectric spheroid core, (H) Multi-nanoparticle structures, (I) Metal nanocube and nanotriangle/nanoprism, or (J) Metal cylinder.

FIG. 37 is a schematic diagram showing an embodiment of the nanoprobe system having a “crescent structure” partially covering a dielectric core (e.g., silica, polymeric material, etc.).

FIG. 38 is a schematic diagram of an exemplary surfactant-free nanostar synthesis method.

FIGS. 39A-H are schematic diagrams showing exemplary embodiments of nanoprobe systems: (A) a Plasmonics-active metal nanostar, (B) a Nanostar labeled with optical dye and/or drug molecules, (C) a Nanostar with layers (embedded with label and/or drug), (D) a Nanostar with layer (embedded with label and/or drug) and protective overlayer, (E) a Nanostar with paramagnetic spherical nucleus, (F) a Nanostar with elongated paramagnetic nucleus, (G) a Void-space nanostar, and (H) a Nanostar with empty or dielectric core.

FIGS. 40A-H are schematic diagrams showing exemplary embodiments of nanoprobe systems with bioreceptors: (A) Plasmonics-active metal nanostars with bioreceptor, (B) Nanostar labeled with optical dye and/or drug molecules with bioreceptor, (C) Nanostars with layer (embedded with label and/or drug) with bioreceptor, (D) Nanostar with layer (embedded with label and/or drug) and protective overlayer with bioreceptor, (E) Nanostar with paramagnetic spherical nucleus with bioreceptor, (F) Nanostar with elongated paramagnetic nucleus with bioreceptor, (G) Void-space nanostars with bioreceptor, (H) Nanostar with empty or dielectric core with bioreceptor.

FIG. 41 is a schematic diagram of the synthesis of folate-targeted theranostic nanoprobes.

FIG. 42 shows Raman/SERS mapping of the three different cell lines after 4 hr incubation with the FA-targeted theranostic nanoprobes at 0.1 nM concentration.

FIG. 43 is a schematic diagram showing exemplary embodiments of molecular systems for targeting various miRNA biotargets.

FIG. 44 is a diagram showing an iMS-ASO-siRNA with LNAs.

FIGS. 45A and 45B are schematic diagrams of photocleavable linkers. FIG. 45(A) illustrates a single photocleavable linker, and FIG. 45(B) illustrates multiple photocleavable linkers within the oligonucleotide backbone.

FIGS. 46A and 46B are schematic diagrams of photo-removable macromolecules or a “molecular lock” mechanism.

FIG. 47 is a schematic diagram of photo-activatable iMS nanoprobes using photo-removable caging groups to inactivate iMS.

FIG. 48A is a diagram showing an exemplary design of an iMS theranostic nanoprobe (iMS-ASO) with the capture probe acting as an antisense oligonucleotide (ASO).

FIG. 48B is a diagram showing an exemplary design of an iMS-ASO for miR-21 using unmodified oligonucleotides.

FIG. 48C is a diagram showing an exemplary design of an iMS-ASO for miR-21 using LNA-modified oligonucleotides.

FIGS. 49A, 49B, 49C are diagrams showing an exemplary design of a dual targeting theranostic nanoprobe containing an iMS nanoprobe and siRNA with the capture probe acting as an antisense oligonucleotide (iMS-ASO-siRNA). In FIG. 49(a), the capture probe (ASO) is linked to siRNA directly. In FIG. 49(b), the capture probe (ASO) is linked to siRNA through a hexa-ethylene glycol (HEG) Spacer. In FIG. 49(c), the capture probe (ASO) is linked to siRNA through a photocleavable linker.

FIG. 50 is a schematic diagram of an exemplary embodiment of a dual targeting system with a miR-21 iMS and a siRNA against p53, a common mutated gene in various diseases.

FIG. 51 is a schematic diagram showing an exemplary embodiment of an iMS-Aptamer Theranostic Nanoprobe for Thrombin.

FIG. 52 is a schematic illustration of an exemplary nanoprobe system against miR-155.

FIG. 53 is a schematic illustration of an exemplary nanoprobe system against miR-208a.

FIGS. 54A, 54B, 54C are schematic diagrams illustrating embodiments of how the nanoprobe system can be used for theranostics applications.

FIGS. 55A-B are SERS maps showing cellular distribution in J774 cells following incubation for four hours with 4-ATP dye-labeled silver nanoparticles.

FIG. 56A is a chart showing the results of testing in Example 2.

FIG. 56B is a chart showing the results of testing in Example 2.

FIG. 57 is a schematic diagram of exemplary SERS nanosensors for in vivo detection of nucleic acid targets in a large animal model.

FIG. 58 is a series of charts showing biosafety data for two of different types of GNS: AuNS@Ag-PEG-TAT compared to AuNS-PEG-TAT.

FIG. 59 is a chart showing results of the GNS particle retention study.

FIGS. 60A-D are images of Raman mapping of plant cells incubated with iMS nanoprobes, and FIG. 60D I is a chart showing the results of testing.

FIGS. 61A-B are images of Raman mapping of plant cells incubated with iMS nanoprobes, and FIG. 61B I is a chart showing the results of testing.

FIGS. 62A and 62B are schematic diagrams showing an exemplary operating procedure using nanoprobe systems for in vivo diagnostics for real time, permanent and continuous ‘health monitor’ of systems (stem cells, tissue-engineered organs, etc.) implanted in a subject (for example, a human subject).

FIG. 63 is an illustration showing the design of iMS nanoprobes targeted to miR-21.

FIG. 64 is a chart showing SERS spectra of the three differently labeled iMS nanoprobes targeting to miR-21, miR-194 and miR-39.

FIG. 65 shows an example of a mixture of the four distinct dyes measured using 633 nm laser excitation for non-resonant SERS.

FIGS. 66A, 66B, and 66C are schematic diagrams illustrating the basic principle of Raman/SERS Hyperspectral Imaging (HSI).

FIG. 67A is a schematic diagram illustrating a hyperspectral data cube showing the spectral information of a series of Raman images collected at various wavelength of interest.

FIG. 67B is a schematic diagram of a noncollinear AOTF device showing the diffraction of unpolarized light into three beams: Two narrow-band diffracted (dark arrows) and a broadband undiffracted beam (white).

FIG. 68 is a chart showing SERS spectra for the exemplary probes.

FIG. 69 is a series of images enabling comparison of two-photon photoluminescence in gold nanostar-versus Qtracker-labeled adipose-derived stem cells.

FIG. 70 is a series of images showing gold nanostars as position sensors and trackers for adipose stem cells.

FIG. 71 is a schematic diagram of 2 embodiments of a TAT peptide funcationalized iMS nanoprobe.

FIG. 72 is a chart showing SERS spectra of the TAT-peptide-functionalized miR-21 iMS nanoprobe (embodiment 1) in the presence (+miR-21 Target) or absence (Blank) of miR-21 target sequences.

FIGS. 73A, 73B, and 73C are schematic diagrams showing an exemplary in vivo diagnostic modality for cellular systems having photo-activated nanoprobes.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments; any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a human who is undergoing a procedure using the systems and methods prescribed herein.

Unless otherwise defined, all technical terms used herein have the same meaning as commonly understood by one of ordinary skill in the art to which this disclosure belongs.

Nanoprobe systems and methods of using them for detection, monitoring, diagnostics and/or therapeutics are described herein. In embodiments, the use of nanoprobe systems for in vivo monitoring, detection, diagnostics and/or treatment are described. When nanoprobe systems are used in vivo, consideration of how the systems will interact with, be taken into or delivered to and distributed in cells is important. In embodiments, the nanoprobe system comprises an attachment mechanism for use in attaching the nanoprobe system to a cell membrane. The attachment mechanism can attach the nanoprobe system inside the cell (intracellular) or outside the cell (extracellular). Techniques for intracellular delivery of nanoprobe systems, as well as providing surface modifications to prevent aggregation and optimize surface-enhanced Raman scattering (SERS) signal strength can be important. For example, targeting of cells may be improved by using targeting proteins/peptides on the nanoprobe surface. An exemplary peptide is the HIV-1 TAT for nuclear entry. Aggregation of nanoprobe systems can be addressed by use of pre-stabilized nanoprobes, which may be coated with poly(ethylene glycol) (PEG) ligands. With regard to plants, synthesized magnetic nanoprobe systems can be delivered into plant cells with and without cell walls using an external magnetic field. Two-dimensional (2D) Raman imaging can identify and locate nanoprobe systems within single cells using SERS. The uptake efficiency of nanoprobe systems in single cells can be monitored via SERS imaging, and uptake efficiency can be enhanced via surface modification with charged or uncharged molecular labels.

As used herein, the term nanoprobe system refers to a system comprising a plasmonic-active nanoparticle (for example, gold nanostars, metallic nanostructures, etc.) and a molecular probe system (for example, an oligonucleotide capable of forming a stem-loop configuration, etc,).

A nanoprobe (also referred to as nanosensor) system comprises a plasmonic-active nanoparticle and a molecular probe system; said molecular probe system comprises an oligonucleotide capable of forming a stem-loop configuration, having a first end and a second end, wherein the oligonucleotide is immobilized to the plasmonic-active nanoprobe at the first end and labeled with a Raman reporter at the second end, and an unlabeled placeholder strand at least partially bound to the oligonucleotide. A nanoprobe system may also comprise a siRNA or other molecular probe systems for use in therapy.

A brief summary of basic plasmonic principles is provided in order to explain how the plasmonic-active nanoparticle is able to provide detection and monitoring capabilities. Electromagnetic enhancements are divided into two main classes: a) enhancements that occur only in the presence of a radiation field, and b) enhancements that occur even without a radiation field. The first class of enhancements is further divided into several processes. Plasma resonances on the substrate surfaces, also called surface plasmons, provide a major contribution to electromagnetic enhancement. An effective type of plasmonics-active substrate consists of nanostructured metal particles, protrusions, or rough surfaces of metallic materials. Incident light irradiating these surfaces excites conduction electrons in the metal, and induces excitation of surface plasmons leading to Raman/Luminescence enhancement. At the plasmon frequency, metal nanoparticles (or nanostructured roughness) become polarized, resulting in large field induced polarizations and thus large local fields on the surface. These local fields increase the luminescence/Raman emission intensity. As a result, the effective electromagnetic field experienced by the analyte molecule on theses surfaces is much larger than the actual applied field. In the electromagnetic models, the luminescence/Raman-active analyte molecule is not required to be in contact with the metallic surface but can be located anywhere within the range of the enhanced local field, which can polarize this molecule. There are two main sources of electromagnetic enhancement: (1) first, the laser electromagnetic field is enhanced due to the addition of a field caused by the polarization of the metal particle; (2) in addition to the enhancement of the excitation laser field, there is also another enhancement due to the molecule radiating an amplified Raman/Luminescence field, which further polarizes the metal particle, thereby acting as an antenna to further amplify the Raman/Luminescence signal. Plasmonics-active metal nanoparticles also exhibit strongly enhanced visible and near-infrared light absorption, several orders of magnitude more intense than conventional laser phototherapy agents. The use of plasmonic nanoparticles as highly enhanced photoabsorbing agents has introduced a much more selective and efficient phototherapy strategy. The tunability of the spectral properties of the metal nanoparticles and the biotargeting abilities of the plasmonic nanostructures make their use promising.

The SERS effect can enhance the efficiency of light emitted (Raman or luminescence) from molecules adsorbed at or near a metal nanostructure's Raman scatter.

The intensity of the normally weak Raman scattering process is increased by factors as large as 10¹³ or 10¹⁵ for compounds adsorbed onto a SERS substrate, allowing for single-molecule detection. As a result of the electromagnetic field enhancements produced near nanostructured metal surfaces, nanoparticles have found increased use as fluorescence and Raman nanoprobes.

When a nanostructured metallic surface is irradiated by an electromagnetic field (e.g., a laser beam), electrons within the conduction band begin to oscillate at a frequency equal to that of the incident light. These oscillating electrons, called “surface plasmons,” produce a secondary electric field which adds to the incident field. If these oscillating electrons are spatially confined, as is the case for isolated metallic nanospheres or roughened metallic surfaces (nanostructures), there is a characteristic frequency (the plasmon frequency) at which there is a resonant response of the collective oscillations to the incident field. This condition yields intense localized field enhancements that can interact with molecules on or near the metal surface. Secondary fields are typically most concentrated at points of high curvature on the roughened metal surface.

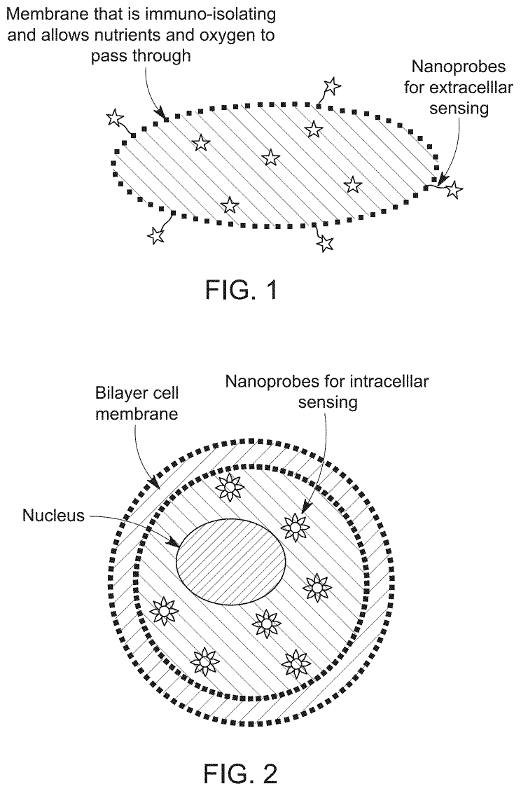

Nanoprobe systems are effective for diagnostic and therapeutic applications because they can attach via attachment mechanism to cell membranes both inside the cell (intracellular) and outside the cell (extracellular) for in vivo use. FIG. 1 shows a schematic diagram of intracellular and extracellular nanoprobe systems attached to a membrane system for in vivo implantation. In embodiments, nanoprobe systems can be encapsulated in a hydrogel scaffold or can be anchored on the surface of a hydrogel through a lipid bilayer derived from cell membrane. For cellular sensing, nanoprobes can be anchored directly in a cell membrane or encapsulated in a thin layer of hydrogel formed on the cell membrane of individual cells for isolating cells from the host immune system. In an exemplary embodiment, nanoprobes can be encapsulated within hydrogels for in vivo implantation. For example, the hydrogel may comprise Poly-HEMA, agar, Poly-NIPAM.

In embodiments, endocytosis can be used as a technique for delivering SERS nanoprobes (NPs) into cells. For example, nanoprobes can be delivered into living cells by modulating the surface charge on the nanoparticles or functionalization with nuclear targeting peptides, such as, for example, HIV-1 protein-derived TAT peptides. Additional nuclear targeting peptides can include, without limitations a glutamate peptide coupled to the N-terminus of the Oct6 NLS, a modified mRNA transporter, and the M9 component of heterogeneous nuclear ribonucleoprotein-A1. Additionally, electroporation can be used to introduce nanoprobes into cells for intracellular sensing. Electroporation applies electrical pulses to induce transient and reversible pores in the cell membrane, which allow exogenous substances like nanoprobes to enter the cells. FIG. 2 is a schematic diagram of exemplary nanoprobes for intracellular sensing.

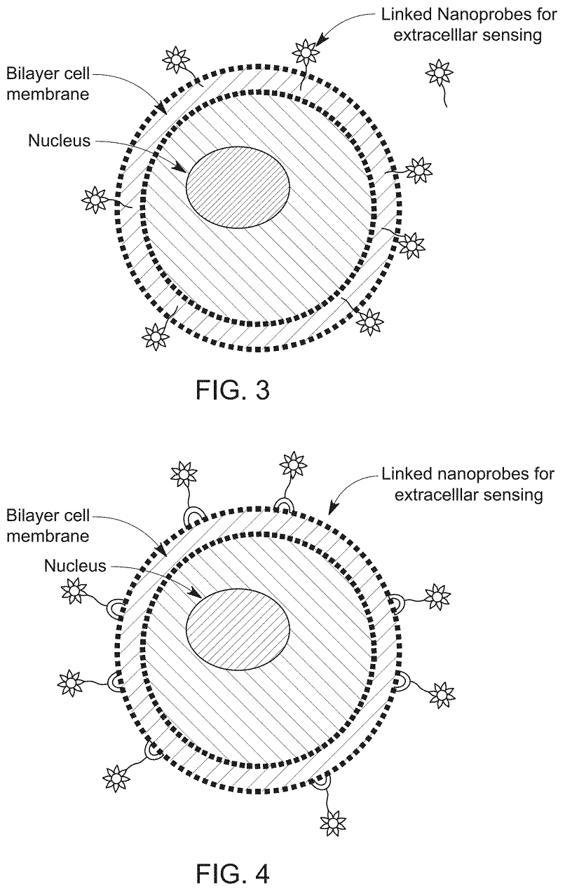

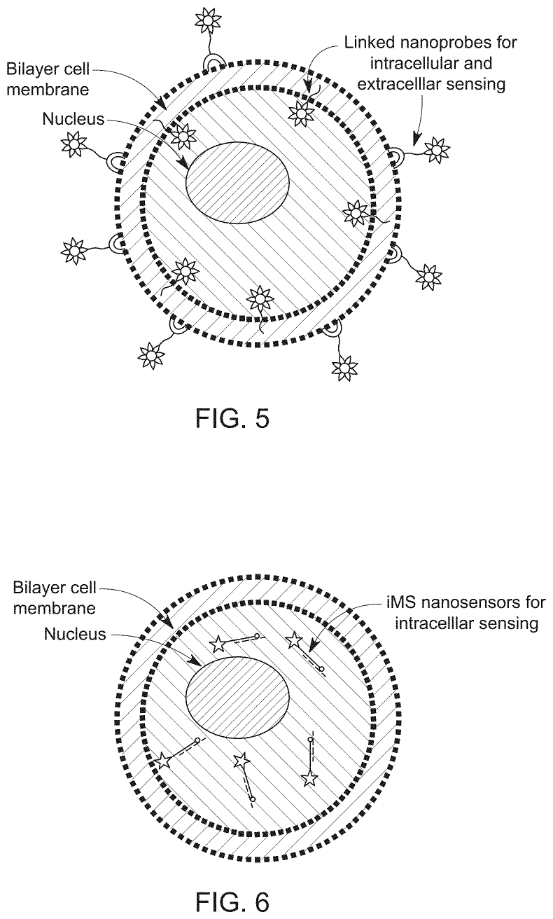

Moreover, in embodiments, nanoprobes can be anchored on cell membranes for extracellular sensing by conjugating nanoparticles with various membrane anchors, including lipids, cholesterol, porphyrin, tocopherol, acyl chain, oleyl chain, or dioleylphosphatidylethanolamine. FIG. 3 is a schematic diagram of exemplary nanoprobes for extracellular sensing. Nanoprobes can also be linked to a cell membrane by functionalizing with antibodies, peptides, or ligands that specifically interact with cell membrane receptors. FIG. 4 is a schematic diagram of an exemplary membrane-linked nanoprobe for extracellular sensing. Nanoprobes can be anchored both inside and outside of a cell membrane for intracellular and extracellular sensing through membrane linkers (as shown in FIG. 3) and cell membrane receptor-specific biomolecules (as shown in FIG. 4). FIG. 5 is a schematic diagram of an exemplary membrane-linked nanoprobe for both intracellular and extracellular sensing.

Inverse molecular sentinel (iMS) nanoprobes, which will be described in greater detail below, can be delivered into or onto living cells in the same way as described above for nanoprobes in general. For example, iMS nanoprobes can be delivered into living cells by modulating the surface charge on the nanoparticles, functionalizing the nanoparticle with nuclear targeting peptides such as HIV-1 protein-derived TAT peptides, and electroporation.

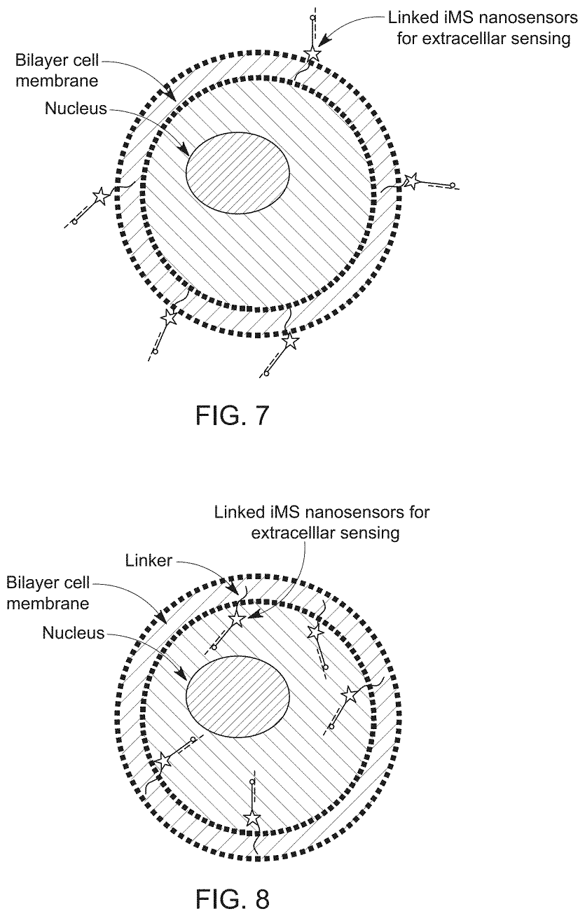

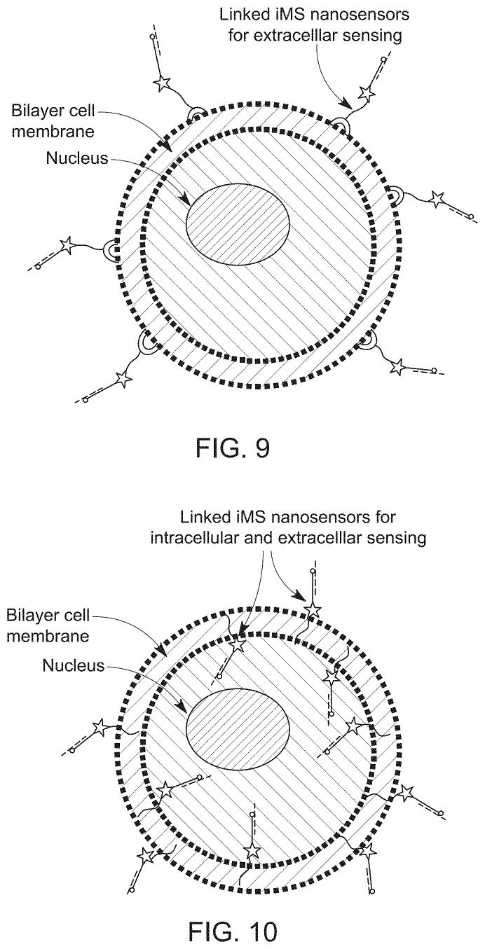

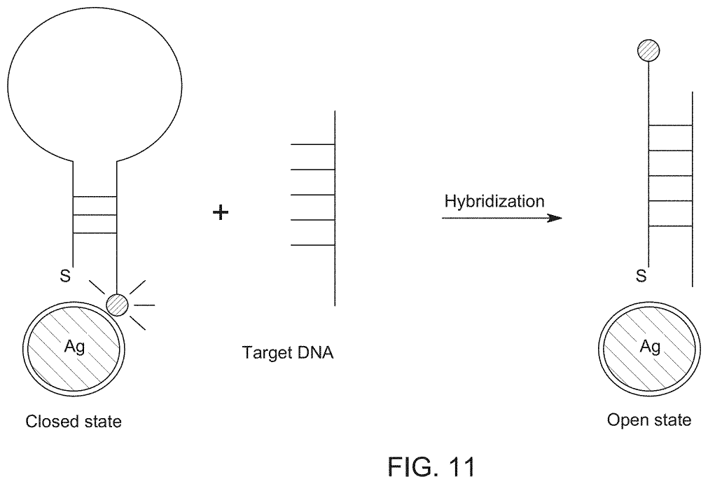

FIGS. 6-10 are exemplary schematic diagrams of iMS nanoprobes being delivered into, onto, or both into and onto living cells. FIG. 6 is a schematic diagram of iMS Nanoprobes for Intracellular Sensing. FIG. 7 is a schematic diagram of membrane-linked iMS nanoprobes for extracellular sensing. FIG. 8 is a schematic diagram of membrane-linked iMS nanoprobes for intracellular sensing. FIG. 9 is a schematic diagram of membrane-linked iMS nanoprobes via receptors (antibody, cell surface receptor, etc.) for extracellular sensing. FIG. 10 is a schematic diagram of membrane-linked iMS nanoprobes for Intracellular and Extracellular Sensing.

Moreover, in embodiments, iMS nanoprobes can be functionalized with membrane anchors, such as lipid, cholesterol, porphyrin, tocopherol, acyl chain, oleyl chain, and dioleylphosphatidylethanolamine, which anchor iMS nanoprobes to the outer cell membrane for extracellular sensing. A membrane-anchor-modified iMS nanoprobe can link to the inner cell membrane for intracellular sensing. Moreover, iMS nanoprobes can be functionalized with antibodies, peptides or ligands that specifically interact with cell membrane receptors for anchoring iMS nanoprobes to the outer cell membrane for extracellular sensing as shown in FIG. 9.

Molecular Sentinel nanoprobes and Inverse Molecular Sentinel nanoprobes will be described in greater detail. “Molecular Sentinel” (MS) nanoprobes are label-free detections system that use SERS for multiplexed detection of gene targets. MS nanoprobes are described in U.S. Pat. No. 7,951,535. FIG. 11 is a schematic diagram showing the operating principle of a “Molecular Sentinel” (MS) nanoprobe. The MS nanoprobe comprises a DNA hairpin probe (30-45 nucleotides) and metal nanoparticles. One end of the hairpin is tagged with a SERS-active label. At the other end, the probe is modified with a thiol group to covalently bond with the nanoparticle. The sequence within the loop region is complementary to the specific target sequence. In the absence of the target, the Raman label is in close proximity (“closed state”) to the metal surface, and a strong SERS signal is detected due to the ‘plasmonic’ enhancement mechanism near the metallic nanoparticle. In the presence of the specific DNA target, hybridization disrupts the stem-loop configuration (“open state”) and separates the Raman label from the metal nanoparticle. The SERS signal is therefore significantly quenched. The sensor turns “off”.

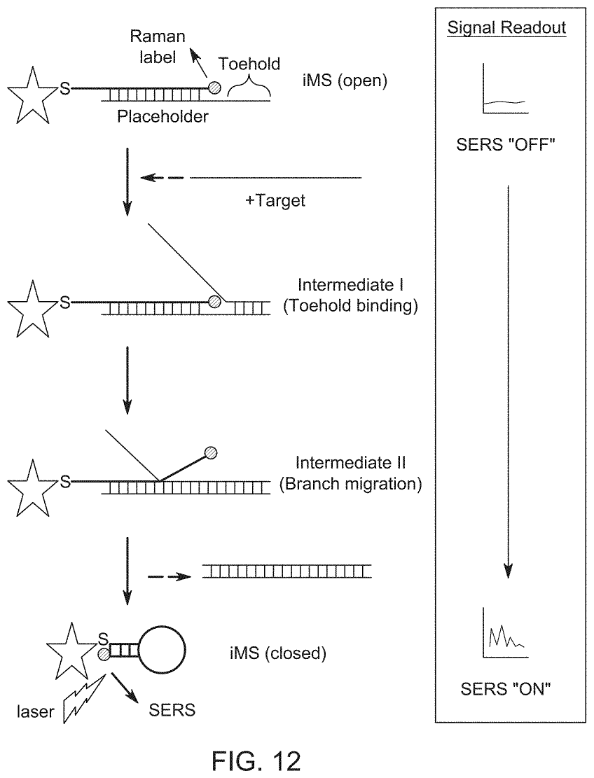

An Inverse Molecular Sentinel (iMS) works in a different way than the MS described above. The nanoprobe system described and used herein is an inverse molecular sentinel system. FIG. 12 is a schematic diagram illustrating an embodiment of a iMS nanoprobe. The iMS nanoprobe system comprises three components: (1) an oligonucleotide capable of forming a stem-loop configuration and labeled with a Raman reporter (Raman label), which provides the source of the Raman signal, (2) a plasmonic-active nanoparticle (e.g. gold nanospheres, nanorods, nanoshells, or nanostars) and (3) an unlabeled placeholder strand or capture probe. The term “placeholder strand” and “capture probe” are used interchangeably herein as the placeholder strand is used to hybridize to or capture the target of interest. The “stem-loop” oligonucleotide of the iMS, having a Raman label at one end of the stem, can be immobilized onto the plasmonic-active nanoparticle, which may be a metallic nanoparticle or nanostar, via a metal-thiol bond. A complementary DNA probe, serving as a “placeholder” strand bound to the iMS nanoprobe, keeps the Raman label away from the nanoparticle surface. The placeholder strand may be partially or completely complementary to the oligonucleotide capable of a stem-loop configuration. In the absence of a target, the nanoprobe system is “open” with very low or no SERS signal (that is, the ‘Off’ state) because the plasmon field enhancement decreases significantly with increasing distance of the Raman label from the metallic surface. Upon exposure to target sequences, the placeholder strand or capture probe leaves the oligonucleotide because of placeholder strand base-pairing with the complementary target strand, following a non-enzymatic strand-displacement process. In the process, the target first binds to a toehold region (intermediate I) of the placeholder and begins displacing the oligonucleotide from the placeholder strand via branch migration (intermediate II), and eventually releases the placeholder strand from the oligonucleotide of the nanoprobe system. Release of the placeholder strand allows the oligonucleotide to fold into the “closed” stem-loop configuration thereby moving the Raman label near or onto the plasmonics-active metal surface, which yields a strong SERS signal. This configuration denotes an ‘On’ status.

In embodiments, the placeholder strand can have a linker to maintain proximity to the nanoprobe after it is released from the oligonucleotide thus enabling the placeholder to be reused.

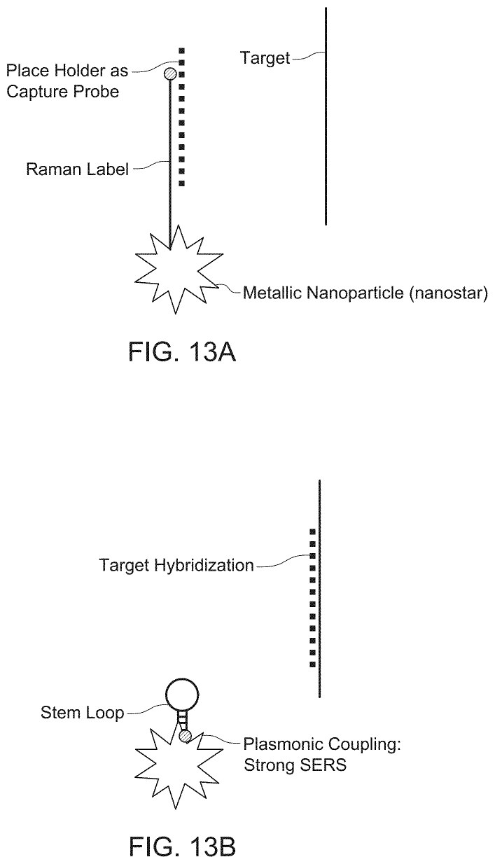

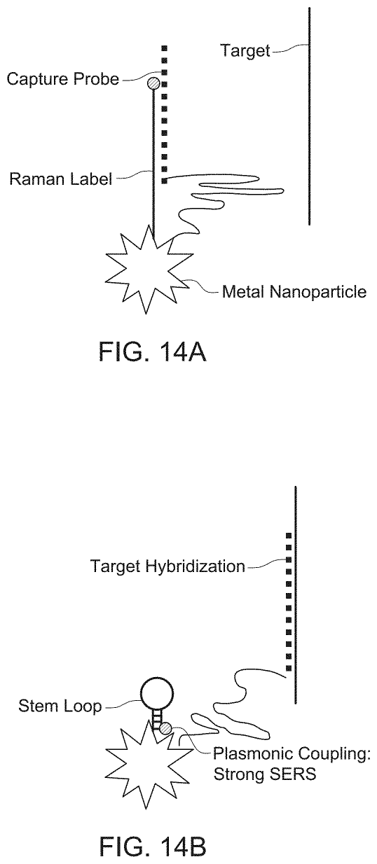

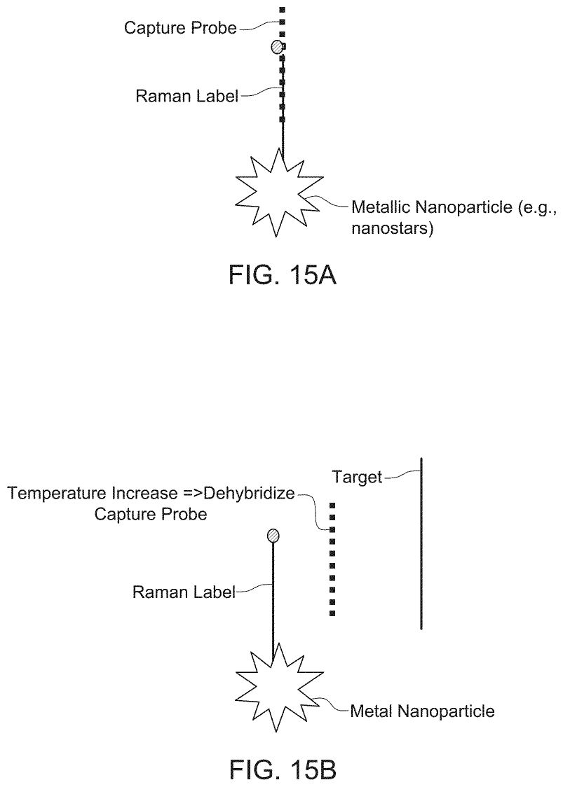

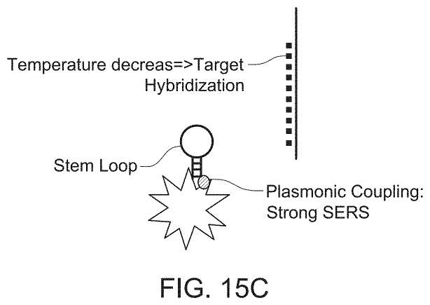

In embodiments, nanoparticles may include, without limitation, silver nanospheres, gold nanospheres, silver and gold nanoshells, and silver and gold nanostars. Such nanoparticles can be used to yield intense SERS signals of the Raman label at different plasmon resonance wavelengths. The “stem-loop” oligonucleotide can have a Raman dye at one end (the first end) as the reporter, and a thiol group at the other end (the second end) for attaching to the nanoparticle. The “stem-loop” oligonucleotide can be designed to comprise a stem duplex region, a spacer and a placeholder binding region. The stem duplex region will allow the stem-loop structure to form after the placeholder-strand binds to the target molecule and leaves the nanoprobe system. The spacer is designed to provide sufficient distance (over 10 nm) between the Raman dye and plasmonics-active nanoparticle surface to reduce background SERS signal when the probe is open. The placeholder binding region binds to the placeholder-strand to prevent the formation of the stem-loop structure in the oligonucleotide. The placeholder-strand is complementary to the placeholder binding region of the oligonucleotide and to the target sequences. FIG. 13 is a schematic diagraming showing an exemplary iMS using a gold nanostar as the nanoparticle platform. FIG. 14 is a schematic diagram showing an exemplary nanoprobe system including a linker. The linker is designed to keep capture probe connected to the nanoprobe system for future re-use in regenerable sensing systems as needed. FIG. 15 is a schematic diagram showing an exemplary iMS nanoprobe system using temperature cycling. As shown, with temperature decrease and in the presence of the target sequence the placeholder strand hybridizes to the target.

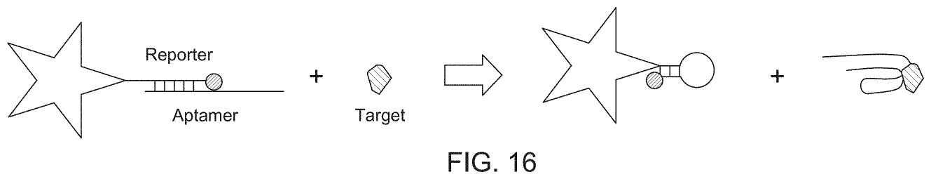

iMS nanoprobe systems may include the use of aptamers. Aptamers are single-stranded DNA or RNA oligonucleotides that can fold into unique structures with high affinity and specificity to their target molecules. Various aptamers have been generated through the process of “systematic evolution of ligands by exponential enrichment” (SELEX) to recognize a wide range of molecules, including proteins, phospholipids, sugars, nucleic acids, as well as small molecules. iMS nanoprobe systems having aptamers (as shown in the FIG. 16) can be used for molecular imaging and detection of various molecules other than nucleic acids. FIG. 16 is a schematic diagram showing integration of an iMS nanoprobe system with an aptamer.

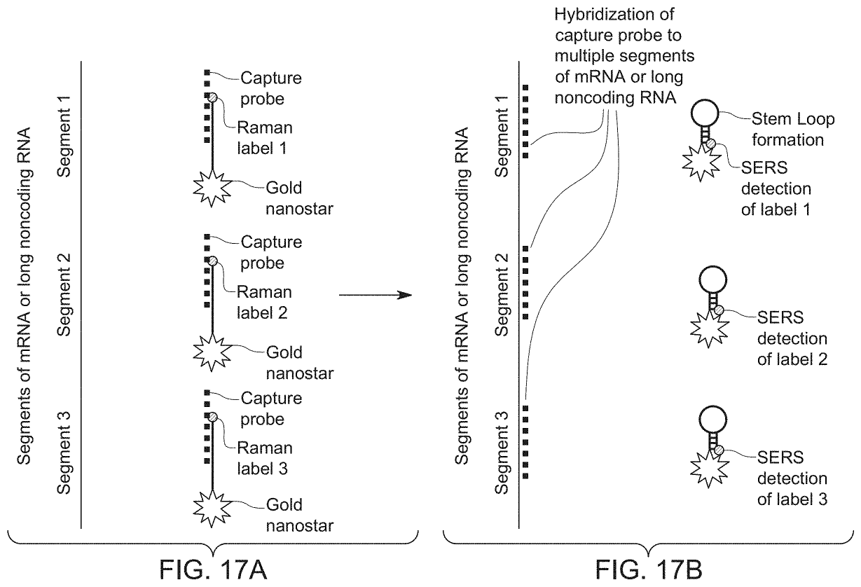

In additional embodiments, an iMS nanoprobe system can serve as an absorber by keeping targets with a capture probe to help remove or decrease the level of target RNA, for example, mRNA, long non-coding RNA (lncRNA) or DNA. In the situation with larger targets, (mRNA, lncRNA, DNA), the iMS nanoprobe system can be designed so that multiple iMS nanoprobe systems, each having shorter sequences, can hybridize cumulatively with the longer mRNA or DNA targets. Each nanoprobe system can have a different Raman/SERS label, thus enabling multiplex detection of multiple targets simultaneously. An additive multiplex approach produces multiple SERS signals associate with the different Raman labels. Spectral analysis of the SERS signals can provide information on the specificity and effectiveness of hybridization. FIG. 17 is a schematic representation of using multiple nanoprobe systems for multiplex detection and blocking (capture) of mRNA and lncRNA.

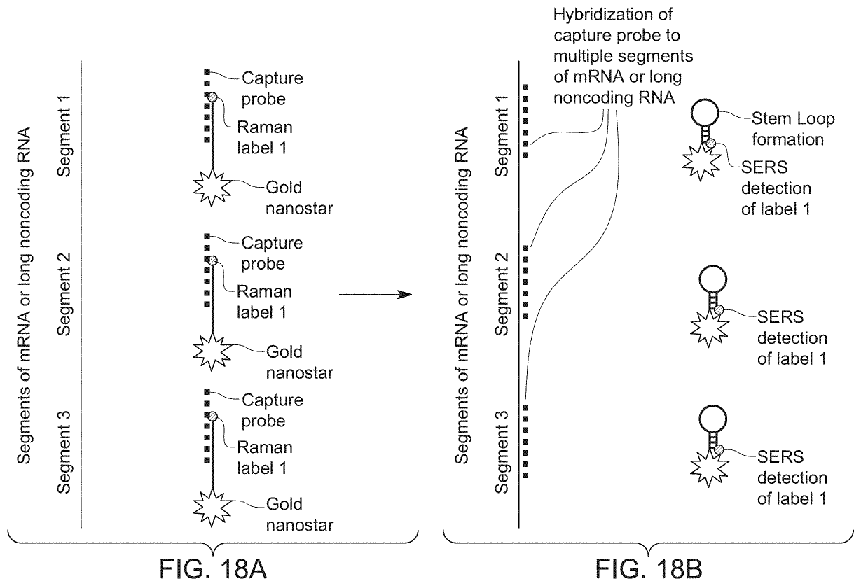

In another embodiment, different nanoprobe systems, having the same Raman label can target multiple segments within the same mRNA or lncRNA target. The presence of targets can be identified from the collective SERS signal from the same Raman/SERS label. This produces an additive effect leading to enhanced total SERS signal and inhibition efficiency. Targeting to multiple segments within the same gene can also reduce the off-target effect. FIG. 18 is a schematic representation of using multiple nanoprobe systems for additive detection and blocking (capture) of mRNA and lncRNA, and therefore, modulating the cellular functions of mRNA, lncRNA as well as miRNAs.

In additional embodiments, an iMS nanoprobe system can further comprise siRNA for therapeutic purposes. Small interfering RNA (siRNA) can regulate the expression of genes, by a phenomenon known as RNAi (RNA interference). The use of siRNA requires ‘carriers’, such as the nanoprobe systems described herein, that can deliver the siRNA to the site of action inside targeted cells. Nanoprobe systems with siRNA can be used for theranostic purposes. As used herein, the term “theranostic or theranostics” includes combined aspects of detecting, monitoring, and/or diagnosing a condition, illness, ailment, disorder and/or disease and treatment or therapy related thereto.

In embodiments, the nanoprobe systems described herein further comprise siRNA thereby enabling the delivery of siRNAs and antisense oligonucleotides, which induce gene regulation (e.g., gene silencing, antisense blocking) and simultaneously produce a signal indicating the gene regulation process. Antisense oligonucleotides are synthetic DNA oligomers that hybridize to target RNA and can be used to induce gene regulation.

Embodiments of the systems, methods and instrumentation described herein can deliver molecular nanoprobe systems comprising various components (e.g., siRNAs, aptamers, or other molecular probes) into cells or outside cells (extracellular matrix, tissues, bodily fluids, etc.) or on cell surface membranes. In embodiments, the nanoprobes described herein can induce molecular regulation mechanisms (e.g., specifically targeted gene silencing, antisense-based blocking, mRNA blocking, miRNA blocking, aptamer-based molecular regulation, etc.) for desired therapy and produce a corresponding sensing signal (theranostics). Such a signal is important for many purposes, such as monitoring the presence of a target and efficiency of treatment.

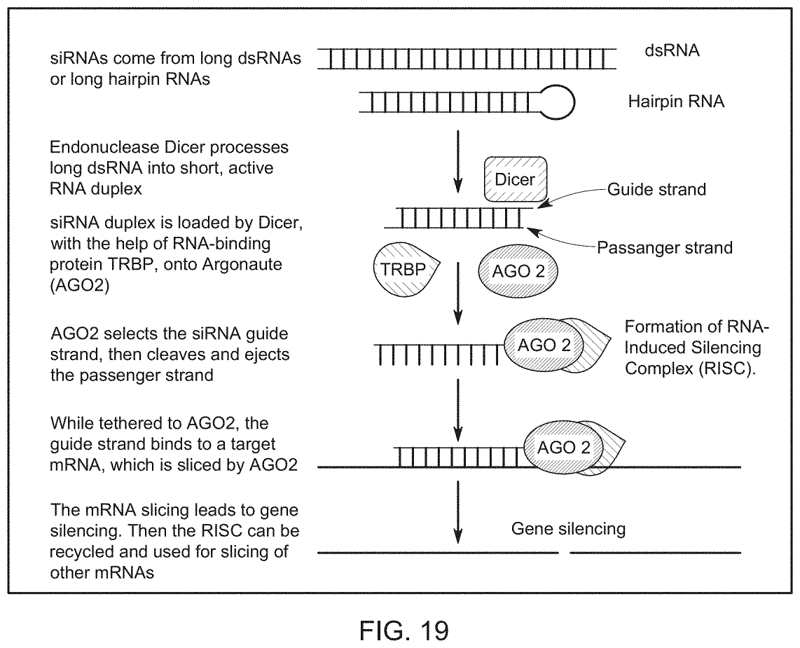

A brief description of siRNA is provided below. FIG. 19 schematically depicts the molecular process of siRNA. siRNA (small interfering RNA) can regulate the expression of genes by selective gene silencing or RNAi (RNA interference). siRNAs originate from long dsRNAs or long hairpin RNAs. Selective gene silencing by RNA interference (RNAi) involves double-stranded small interfering RNA (ds siRNA). The endonuclease Dicer processes long dsRNA into a short, active RNA duplex composed of a single-stranded (ss) guide strand and a passenger strand. Then the siRNA duplex is loaded by Dicer, with the help of RNA-binding protein (TRBP), onto Argonaute (AGO2). AGO2 selects the siRNA guide strand, then cleaves and ejects the passenger strand, which is degraded, thus leading to the formation of an RNA-Induced Silencing Complex (RISC). In the RISC, the guide strand is exposed for base-pairing with its homologous mRNA target, which is then sliced by AGO2. The mRNA slicing leads to gene silencing. Then the RISC can be recycled and used for slicing of other mRNAs.

The concept of using small interfering RNA (siRNA) also includes single-stranded antisense RNA (ss-siRNA), because single-stranded antisense siRNAs also inhibit gene expression.

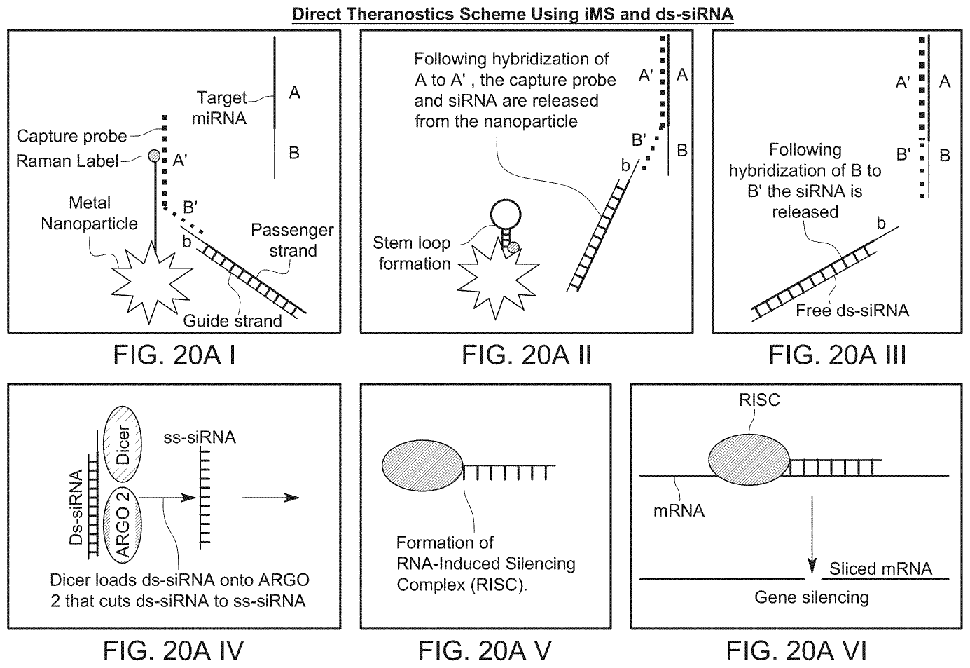

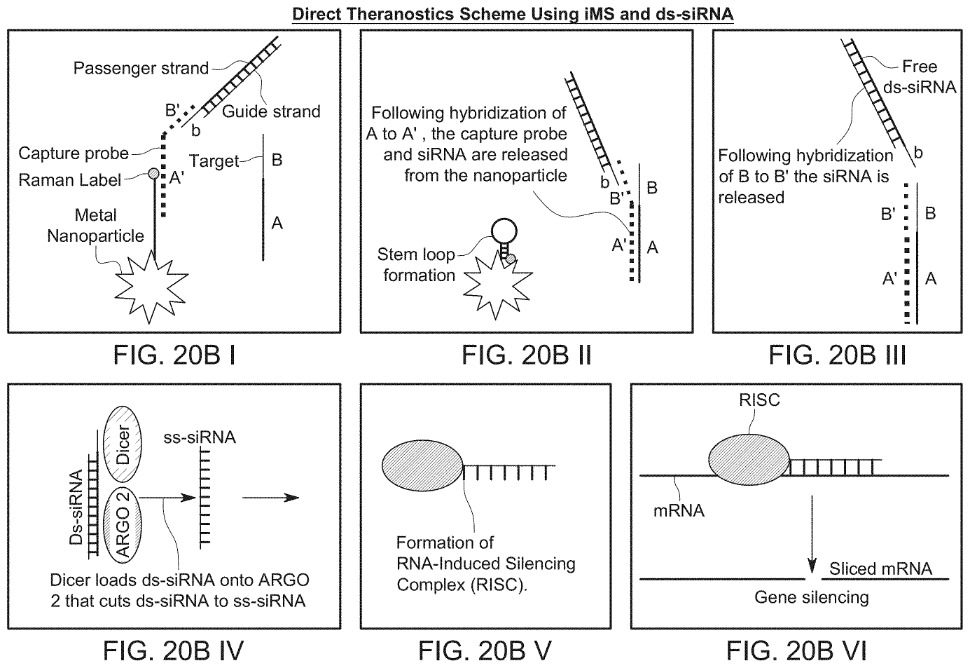

An iMS nanoprobe system comprising siRNA can be used to detect target nucleotides and induce molecular regulation systems. In embodiments, the siRNA can be double strand siRNA (ds-siRNA) or single strand (ss-miRNA). Detection of nucleotides and/or induction of molecular regulations systems can be direct (i.e., take place without outside or additional stimuli) or can be brought about through an external stimulus, such as light (e.g., photocleavage) or heat. Exemplary embodiments comprising siRNA are represented by the schematic diagrams shown in FIGS. 20-32.

FIG. 20 is a schematic diagram showing a Direct Theranostics Scheme using an iMS nanoprobe system and ds-siRNA. The exemplary iMS nanoprobe system comprises a metal nanostar, an oligonucleotide capable of forming a stem loop configuration and having a Raman label, and a placeholder strand having a double strand siRNA that includes a passenger strand and a guide strand. In FIGS. 20-27 and 34, the target is depicted as microRNA. Although not shown, exemplary targets also include mRNA or long non-coding RNA (lncRNA). The placeholder with siRNA hybridizes to the target and the oligonucleotide forms a stem loop configuration whereby the Raman label is at or near the metal nanoparticle and a SERS signal is emitted. Following complete hybridization of the placeholder strand to the target, the siRNA is released, which ultimately leads to sliced mRNA and gene silencing.

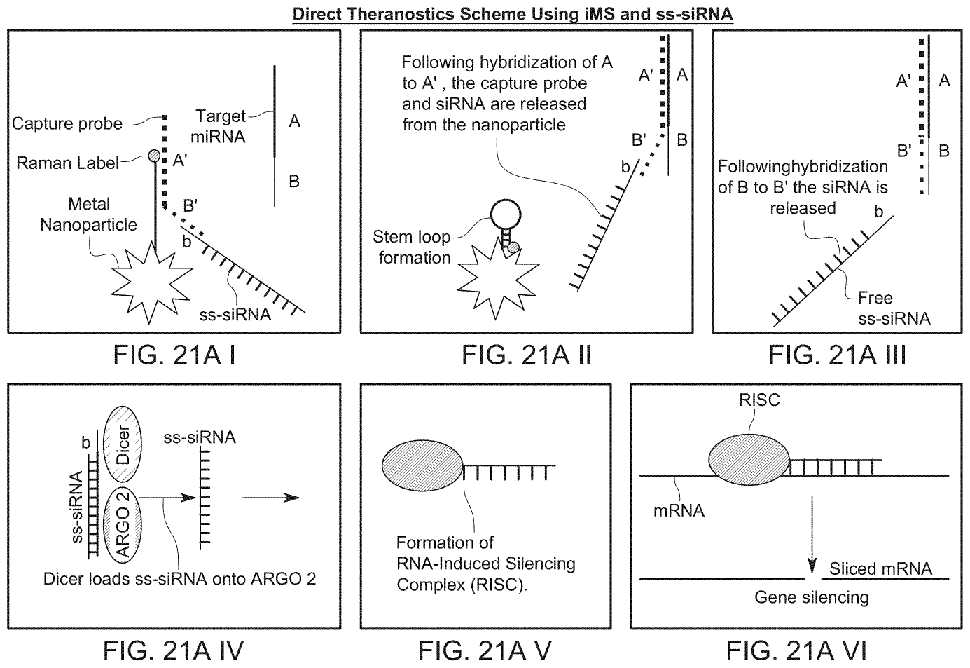

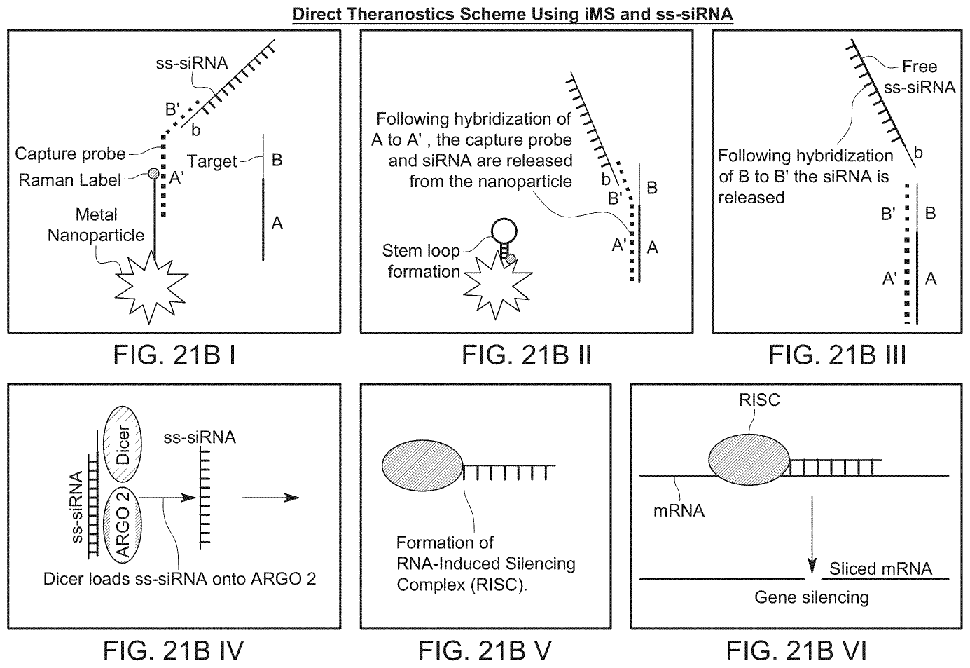

FIG. 21 is a schematic diagram showing a Direct Theranostics Scheme using an iMS nanoprobe system and ss-siRNA. The exemplary iMS nanoprobe system comprises a metal nanostar, an oligonucleotide capable of forming a stem loop configuration and having a Raman label, and a placeholder strand having a single strand siRNA. As stated above, for FIGS. 20-27 and 34, the target is miRNA. The target could also be mRNA or lncRNA. The placeholder with siRNA hybridizes to the target and the oligonucleotide forms a stem loop configuration whereby the Raman label is at or near the metal nanoparticle and a SERS signal is emitted. Following complete hybridization of the placeholder strand to the target, the siRNA is released, which ultimately leads to sliced mRNA and gene silencing.

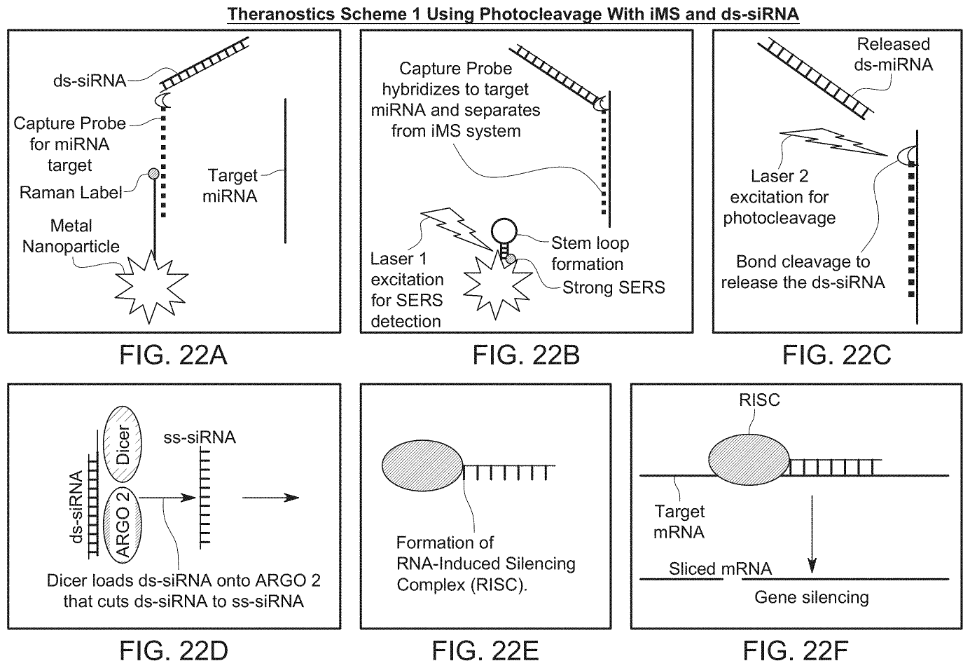

FIG. 22 is a schematic diagram of a Theranostics system using Photocleavage as a stimulus with iMS and ds-siRNA. The exemplary iMS nanoprobe system comprises a metal nanostar, an oligonucleotide capable of forming a stem loop configuration and having a Raman label, and a placeholder strand having a double strand siRNA. The target is miRNA. The placeholder with siRNA hybridizes to the target and the oligonucleotide forms a stem loop configuration whereby the Raman label is at or near the metal nanoparticle and a SERS signal is emitted. Laser excitation is used to emit a SERS signal. Following complete hybridization of the placeholder strand to the target, laser excitation can be used for photocleavage to release the siRNA is, which ultimately leads to sliced mRNA and gene silencing.

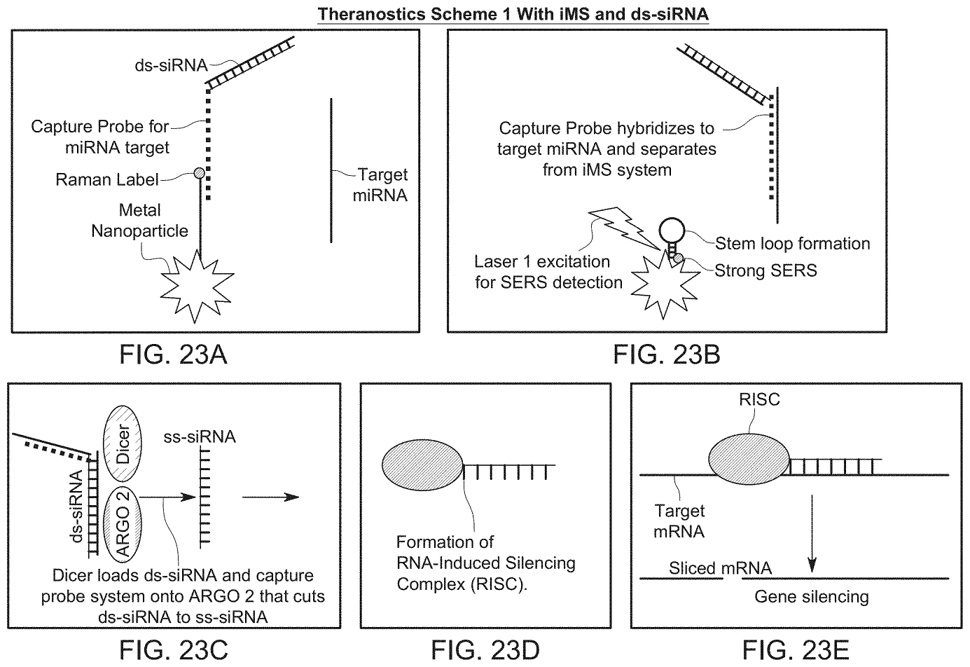

FIG. 23 shows a schematic diagram of a Theranostics system with iMS and ds-siRNA, with no photocleavage. The exemplary iMS nanoprobe system comprises a metal nanostar, an oligonucleotide capable of forming a stem loop configuration and having a Raman label, and a placeholder strand having a single strand siRNA. The target is miRNA. The placeholder with siRNA hybridizes to the target and the oligonucleotide forms a stem loop configuration whereby the Raman label is at or near the metal nanoparticle and a SERS signal is emitted. Laser excitation is used to emit a SERS signal. Following complete hybridization of the placeholder strand to the target, the siRNA is released, which ultimately leads to sliced mRNA and gene silencing.

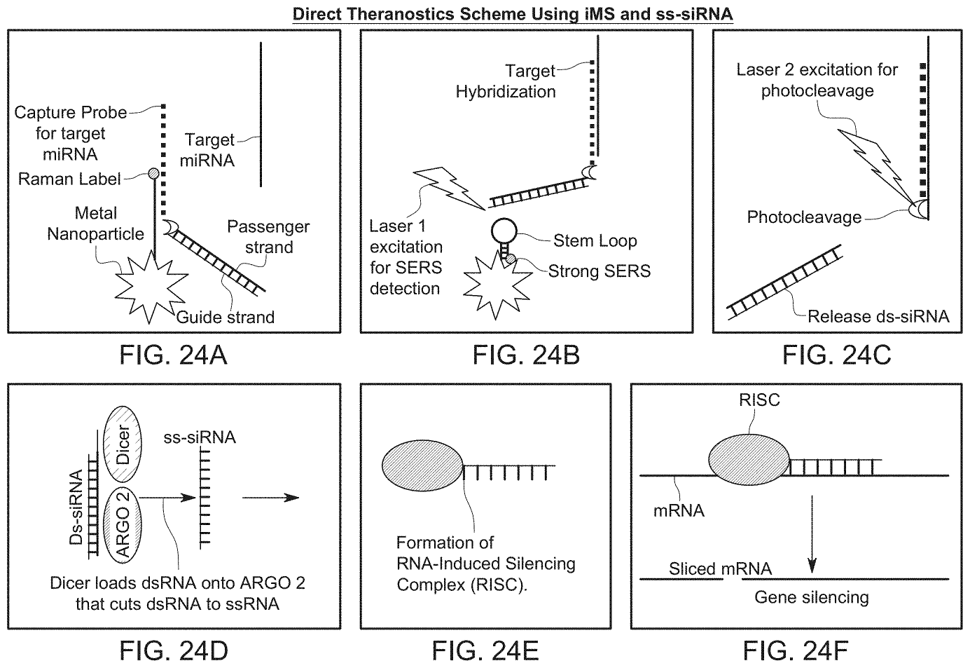

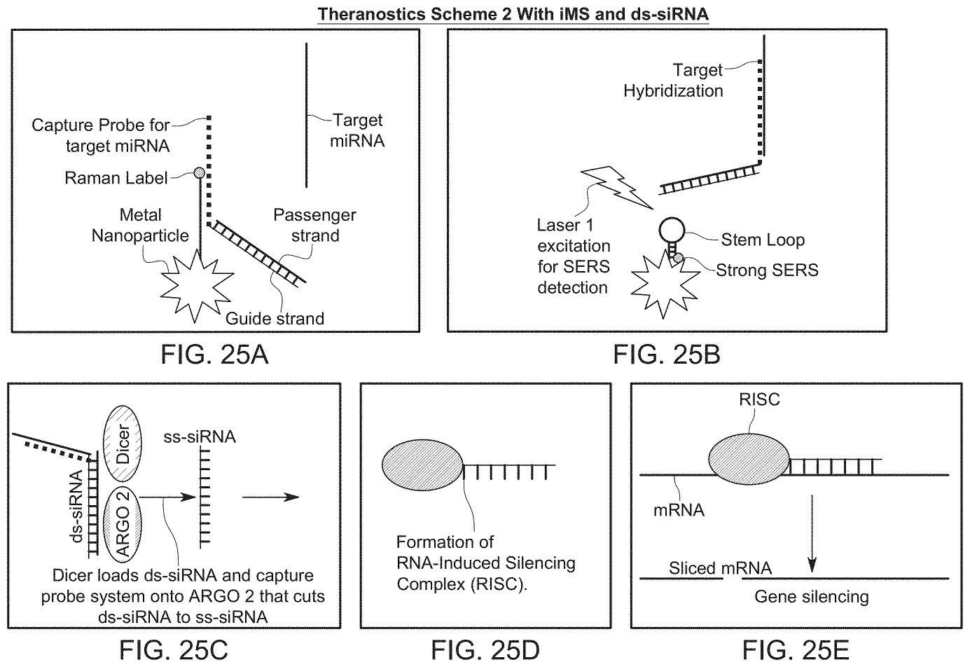

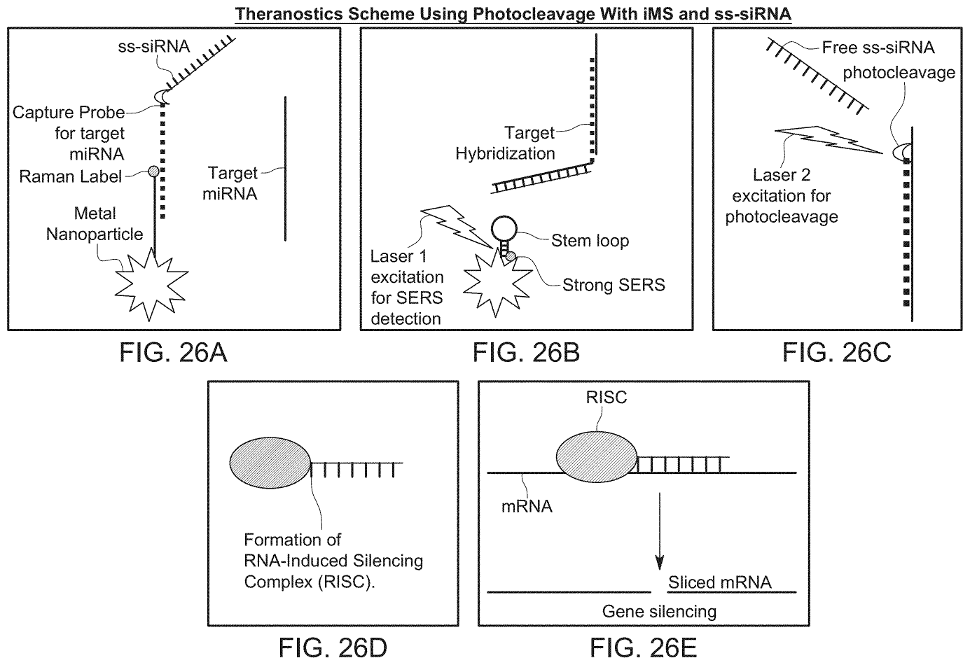

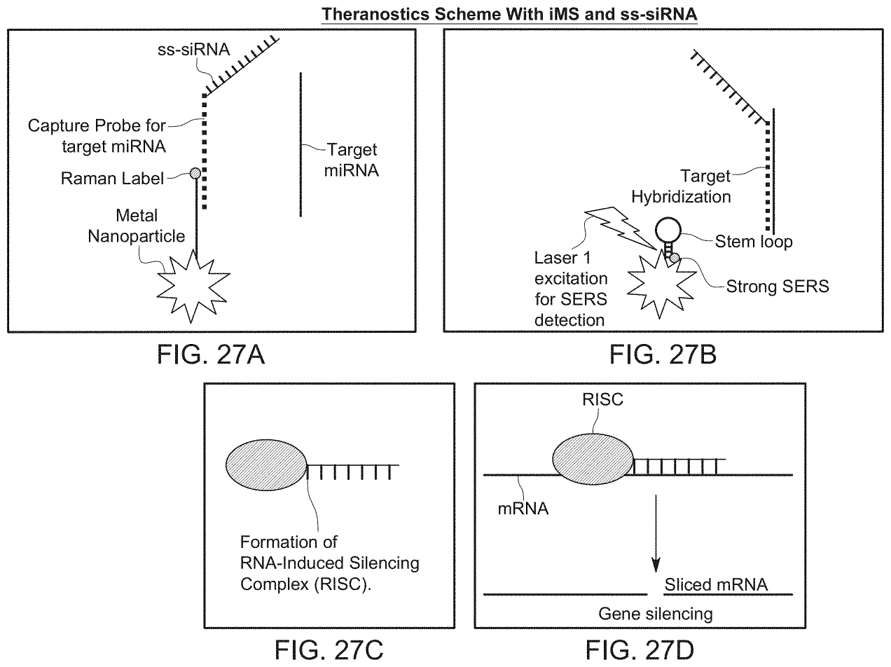

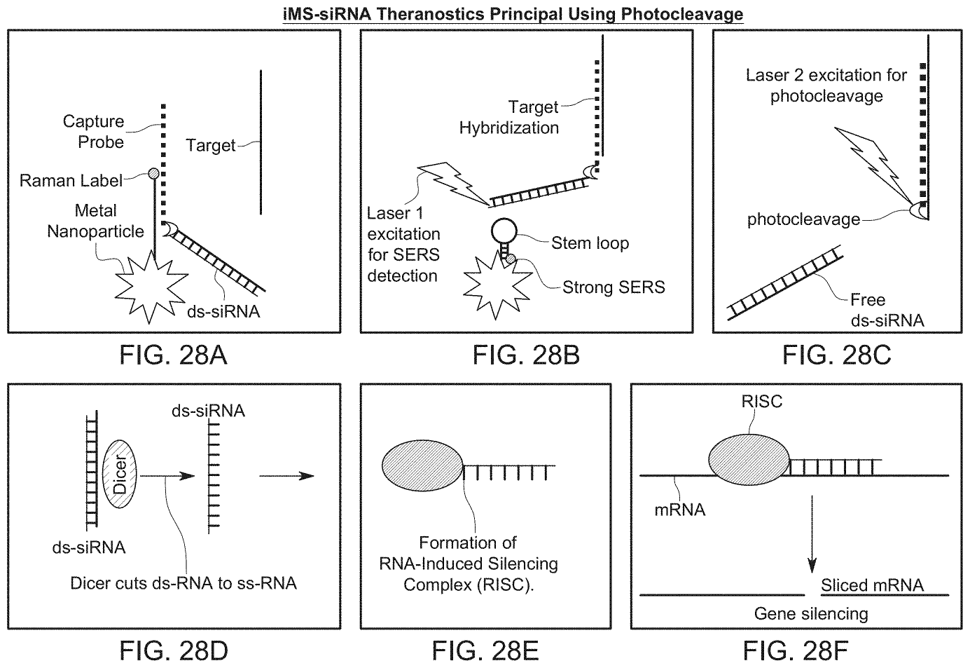

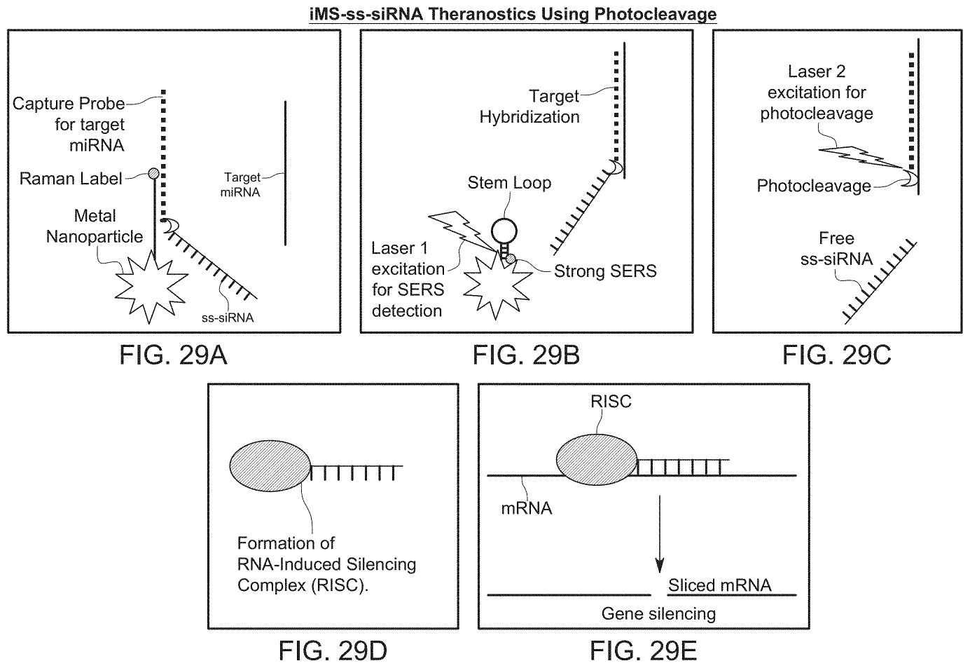

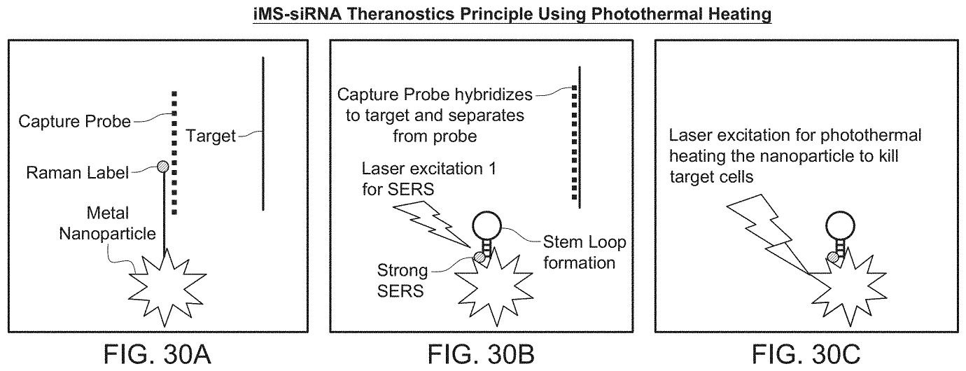

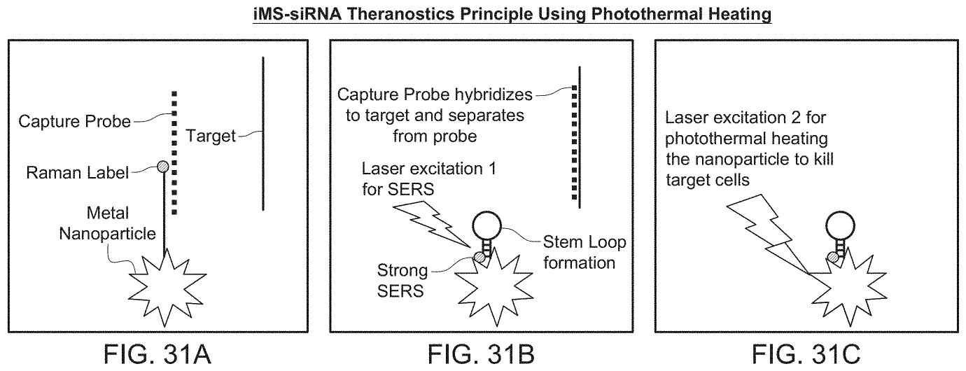

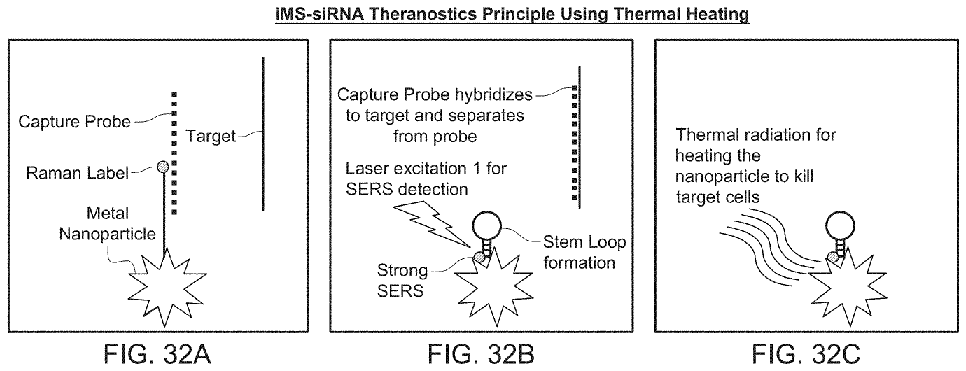

FIG. 24 is a schematic diagram showing a different exemplary embodiment of a Theranostics system using Photocleavage with iMS and ds-siRNA. FIG. 25 is a schematic diagram of a different exemplary embodiment of a Theranostics system with iMS and ds-siRNA. FIG. 26 is a schematic diagram showing an alternative exemplary embodiment of a Theranostics system Using Photocleavage with iMS and ss-siRNA. FIG. 27 shows a schematic diagram of an alternative exemplary embodiment a Theranostics system with iMS and ss-siRNA. FIG. 28 shows a schematic diagram of an alternative exemplary embodiment a MS-siRNA Theranostics system with ds-siRNA and Alternative linker Using Photocleavage. FIG. 29 shows a schematic diagram of an alternative exemplary embodiment a iMS-siRNA Theranostics system with ss-siRNA Using Photocleavage. FIG. 30 shows a schematic diagram of an alternative exemplary embodiment a Theranostics system Using Photothermal Heating with one laser, first for exciting the SERS signal with low laser energy and later to photothermally heat the gold nanostar to kill the target cell with higher energy. FIG. 31 shows a schematic diagram of an alternative exemplary embodiment a Theranostics system using Alternative Photothermal Heating with two different lasers, one laser for exciting the SERS signal and the other laser to photothermally heat the gold nanostar to kill the target cell. FIG. 32 shows a schematic diagram of an alternative exemplary embodiment a MS-siRNA Theranostics system Using Thermal Heating.

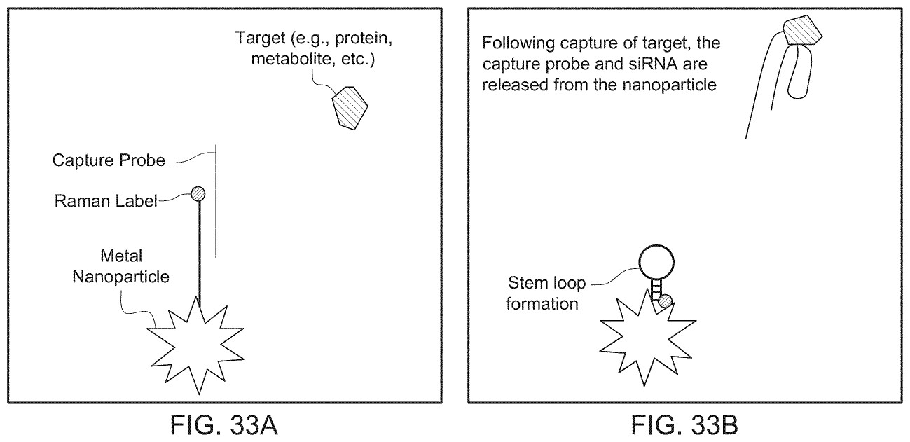

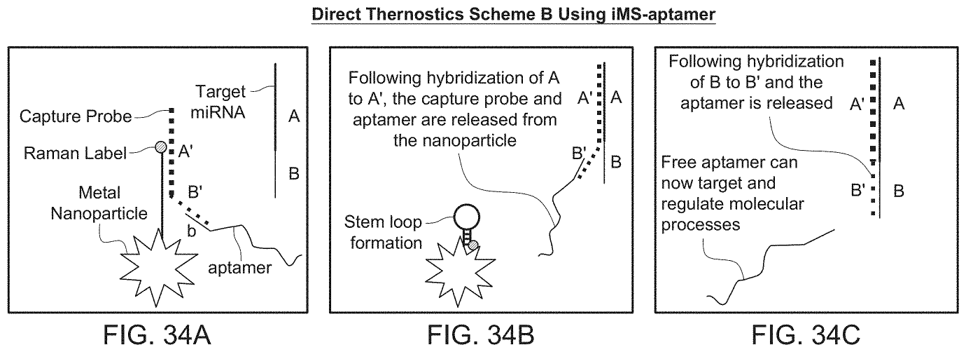

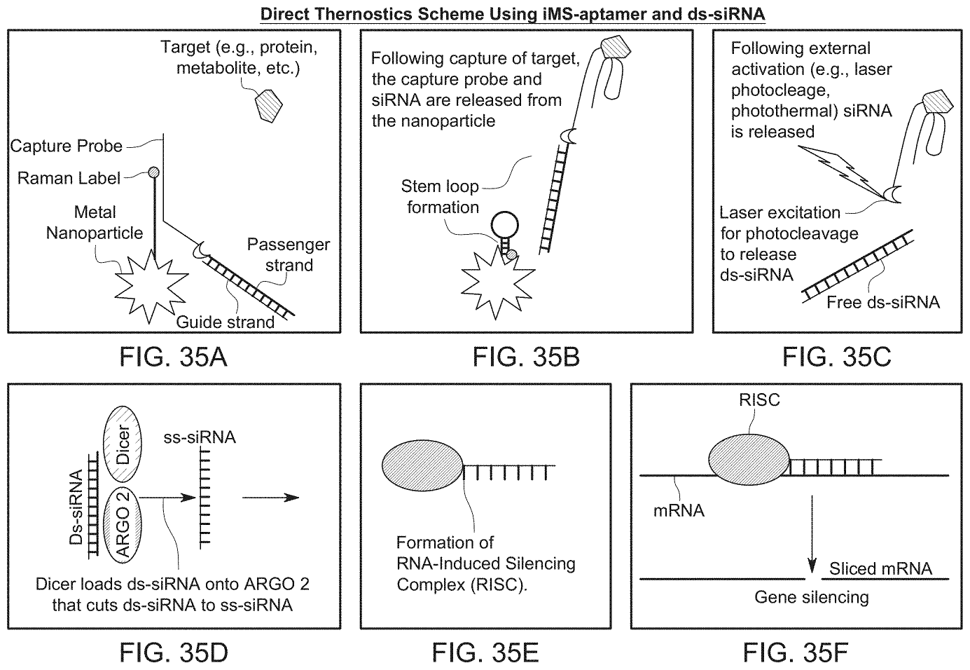

In embodiments, the nanoprobe system can comprise both siRNA and an aptamer. FIG. 33 shows a schematic diagram of an exemplary embodiment of a Theranostics system comprising both siRNA and an aptamer. In the embodiment, the capture probe or placeholder strand is designed as an aptamer with a stronger binding affinity to its target than to the oligonucleotide capable of a stem-loop configuration. The aptamer can bind to its target either inside a cell or on a cell membrane. The aptamer can have a siRNA linked thereto. FIG. 34 provides another exemplary schematic of a nanoprobe system having an iMS-aptamer. In this example, the detection of a target miRNA will trigger a release of an aptamer that is designed to regulate molecular processes for a desired therapy. FIG. 35 is another exemplary schematic of a nanoprobe system. The system shown in FIG. 35 includes iMS, aptamer and ds-siRNA stimulated with photocleavage.

Nanoparticles in the nanoprobe systems can have different shapes and configurations. For example, nanoprobes may have semi-nanoshells and nanoshells, which are partial or complete metallic coatings on a nanoparticle. For example, a semi-nanoshell can be a coating of silver on one side (nanocaps or half-shells) of a nanoparticle. Plasmon resonance of a shell can be tuned by controlling the shell thickness. The shells typically are constructed of a metallic layer over a dielectric core. Prolate and oblate spheroidal shells have been studied. The spheroidal shell has two degrees of freedom for tuning: the shell thickness and the shell aspect ratio. Additionally, the nanoparticle can have a star-shaped structure. Nanostars can be plasmonics-active and induce strong SERS signals.

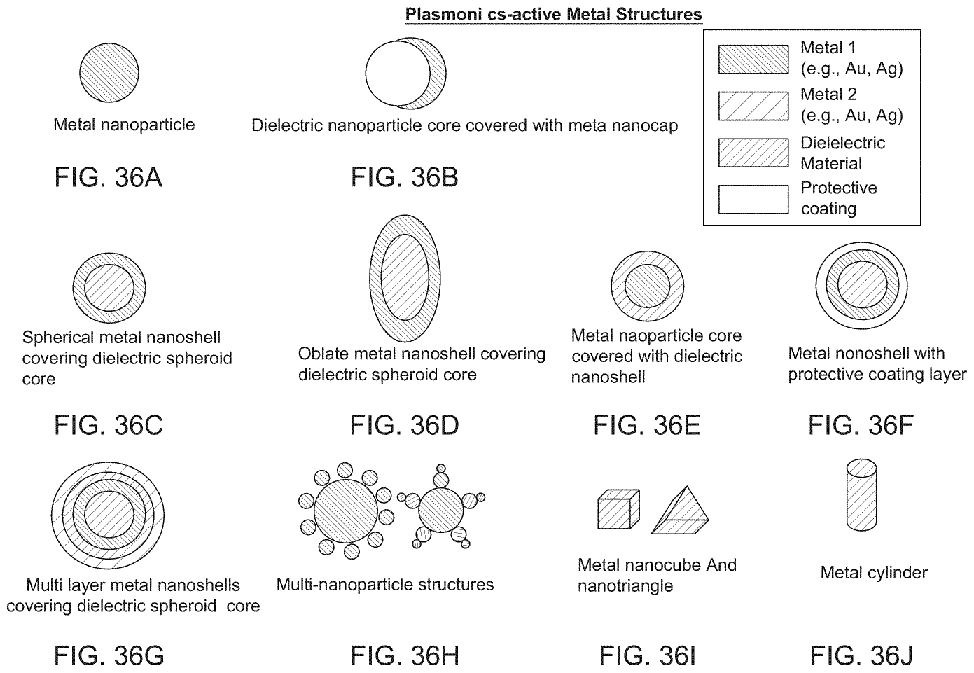

FIG. 36 shows exemplary embodiments of plasmonics-active nanostructures: (A) Metal nanoparticle, (B) Dielectric nanoparticle core covered with metal nanocap, (C) Spherical metal nanoshell covering dielectric spheroid core, (D) Oblate metal nanoshell covering dielectric spheroid core, (E) Metal nanoparticle core covered with dielectric nanoshell, (F) Metal nanoshell with protective coating layer, (G) Multi-layer metal nanoshells covering dielectric spheroid core, (H) Multi-nanoparticle structures, (I) Metal nanocube and nanotriangle/nanoprism, or (J) Metal cylinder.

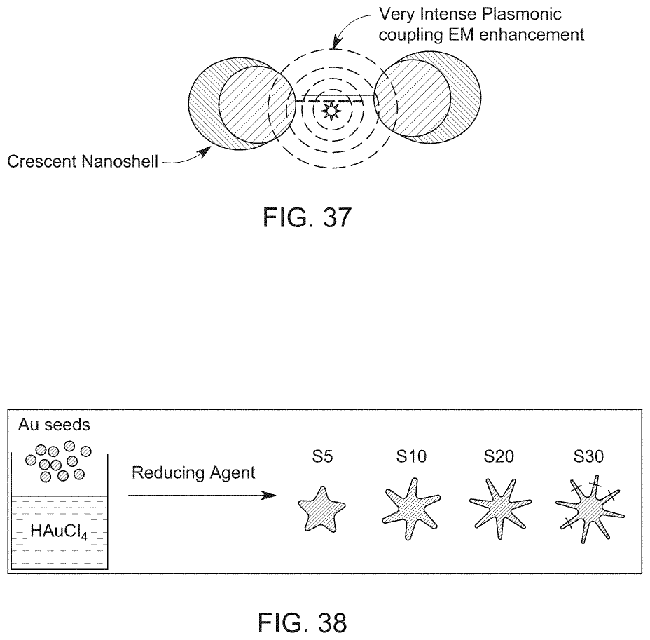

FIG. 37 is a schematic diagram showing an embodiment of the nanoprobe system having a “crescent structure” partially covering a dielectric core (e.g., silica, polymeric material, etc.). The side of the crescent end produces strong plasmonics enhancement. Furthermore, the plasmonic coupling between the crescent—induced enhancements produces a strong SERS effect.

Nanostars of different plasmonic properties can be synthesized using a surfactant-free seed-mediated growth method to evaluate SERS intensity. FIG. 38 is a schematic diagram of an exemplary surfactant-free nanostar synthesis method.

By controlling the geometry, the nanostar plasmon can be tuned to match the excitation laser frequency. To achieve superior SNR and scattering background in tissue, both the laser wavelength working range and the nanostar plasmon will be optimized experimentally. The size of nanostars can be controlled or manipulated by adding different amounts of Au seeds during synthesis: the more seeds added, the smaller the final nanostars size.

Nanostars can exhibit superior SERS properties because of their tunable plasmon, for matching the excitation wavelength, and multiple sharp branches, each with a strongly enhanced electromagnetic field localized at its tip (i.e. “lightning rod” plasmonic effect). The largest E-field enhancement occurs at the tips of the branches of the star. Nanostars size and shape can be tuned based on end use application. That is nanostars (e.g., gold nanostars) of varying sizes and numbers of branches can be synthesized to generate the most intense SERS signal for a given label.

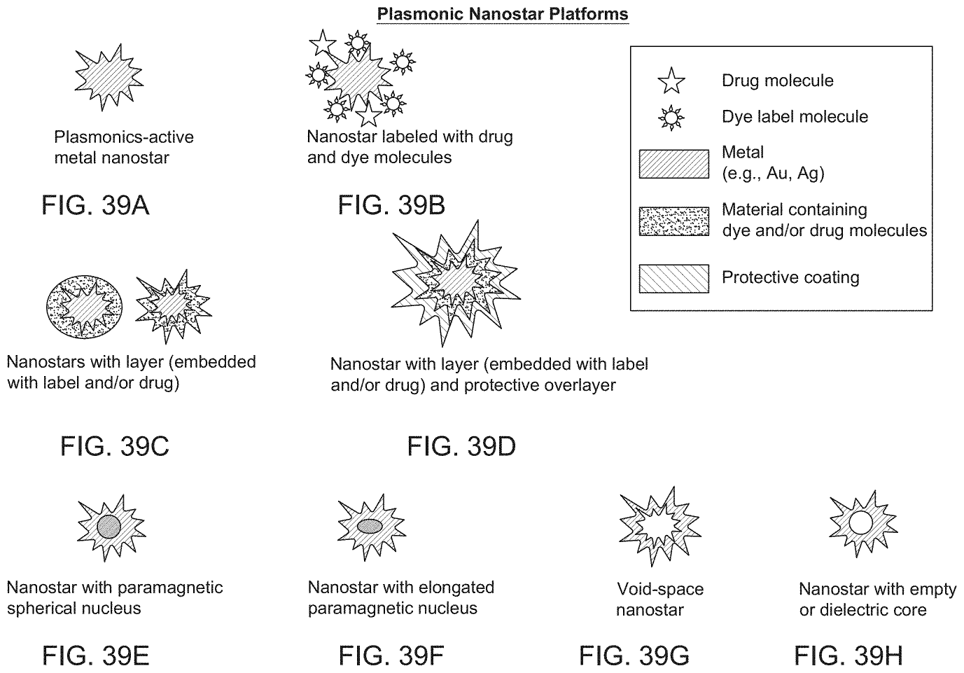

Many embodiments of nanoprobe systems are envisaged. Some exemplary embodiments are shown in FIG. 39. The exemplary embodiments in FIG. 39 include: (A) a Plasmonics-active metal nanostar, (B) a Nanostar labeled with optical dye and/or drug molecules, (C) a Nanostar with layers (embedded with label and/or drug), (D) a Nanostar with layer (embedded with label and/or drug) and protective overlayer, (E) a Nanostar with paramagnetic spherical nucleus, (F) a Nanostar with elongated paramagnetic nucleus, (G) a Void-space nanostar, and (H) a Nanostar with empty or dielectric core.

The nanoprobe systems described herein include a bioreceptor. The bioreceptor generally includes the oligonucleotide and the capture probe or placeholder strand of the nanoprobe system. Bioreceptors are the key to specificity for targeting disease cells or mutated genes or specific biomarkers. Bioreceptors can take many forms. However, bioreceptors can generally be classified into five different major categories. The categories include: 1) antibody/antigen, 2) enzymes, 3) nucleic acids/DNA, 4) cellular structures/cells and 5) biomimetic (aptamers, peptides, etc.).

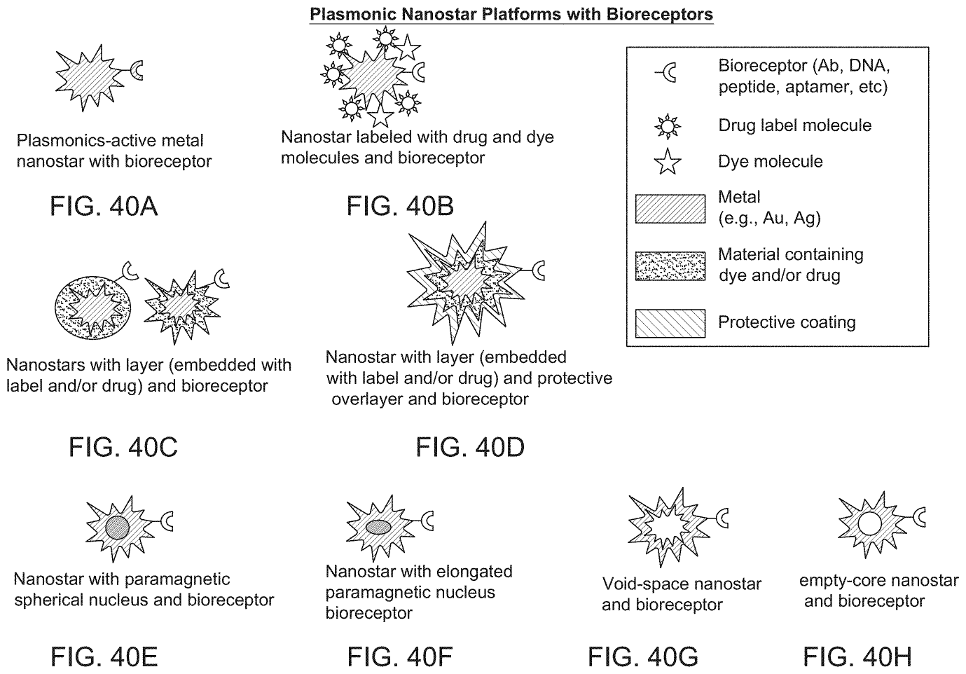

FIG. 40 provides a schematic diagram of various exemplary embodiments of nanoprobe systems with bioreceptors: (A) Plasmonics-active metal nanostars with bioreceptor, (B) Nanostar labeled with optical dye and/or drug molecules with bioreceptor, (C) Nanostars with layer (embedded with label and/or drug) with bioreceptor, (D) Nanostar with layer (embedded with label and/or drug) and protective overlayer with bioreceptor, (E) Nanostar with paramagnetic spherical nucleus with bioreceptor, (F) Nanostar with elongated paramagnetic nucleus with bioreceptor, (G) Void-space nanostars with bioreceptor, (H) Nanostar with empty or dielectric core with bioreceptor.

Bioreceptors can include DNA. Biologically active gene probes (i.e., DNA) can be directly or indirectly immobilized onto a nanoparticle surface to ensure optimal contact and maximum binding. When immobilized onto gold nanoparticles, the gene probes are stabilized and, therefore, can be reused repetitively. Several methods can be used to bind DNA to different supports.

Bioreceptors can include Aptamers. Aptamers are oligonucleotide or peptide molecules that bind to a specific target molecule. Aptamers are usually created by selecting them from a large random sequence pool, but natural aptamers also exist in riboswitches. Aptamers can be combined with ribozymes to self-cleave in the presence of their target molecule.

Bioreceptors can include Antibody Probes. Antibodies are biological molecules that exhibit very specific binding capabilities for specific structures. An antibody is a complex biomolecule, made up of hundreds of individual amino acids arranged in a highly ordered sequence. For an immune response to be produced against a particular molecule, a certain molecular size and complexity are necessary: proteins with molecular weights greater than 5000 Da are generally immunogenic. The way in which an antigen and its antigen specific antibody interact may be understood as analogous to a lock and key fit, by which specific geometrical configurations of a unique key enables it to open a lock. In the same way, an antigen specific antibody “fits” its unique antigen in a highly specific manner. This unique property of antibodies is the key to their usefulness in immunosensors where only the specific analyte of interest, the antigen, fits into the antibody binding site.

Bioreceptors can include Enzyme Probes. Enzymes are often chosen as bioreceptors based on their specific binding capabilities as well as their catalytic activity. In biocatalytic recognition mechanisms, the detection is amplified by a reaction catalyzed by macromolecules called biocatalysts. With the exception of a small group of catalytic ribonucleic acid molecules, all enzymes are proteins. The catalytic activity provided by enzymes allows for lower limits of detection than would be obtained with common binding techniques. The catalytic activity of enzymes depends upon the integrity of their native protein conformation. If an enzyme is denatured, dissociated into its subunits, or broken down into its component amino acids, its catalytic activity is destroyed. Enzyme-coupled receptors can also be used to modify the recognition mechanisms.

Bioreceptors can include Protein-catalyzed capture agents (PCCs). PCCs are synthetic and modular peptide-based affinity agents that can be used as receptors.

In embodiments of the nanoprobe system, silver nanoparticles are used. The surface of the silver nanoparticle can be functionalized to enable more stable immobilizing of the bioreceptor to the nanoparticle. For example, the Ag surface can functionalized with alkylthiols, which form stable linkages. Alkylthiols readily form self-assembled monolayers (SAM) onto silver surfaces in micromolar concentrations. The terminus of the alkylthiol chain can then be directly used to bind biomolecules, or can be easily modified to do so. The length of the alkylthiol chain can keep the biomolecules away from the surface of the nanoparticle. Furthermore, to avoid direct, non-specific DNA adsorption onto the surface of the nanoparticle, alkylthiols can be used to block further access to the surface, allowing only covalent immobilization through the linker.

Moreover, the surface of the nanoparticle can also be functionalized with folic acid. Folic acid (FA) is one of the most common targeting ligands employed for nanoparticle delivery. Many cancer cells overexpress the folate receptor, while normal cells typically have little to no folate receptor expression. By functionalizing the surface of nanoparticles with FA, they can be used to specifically label FR-positive cells for detection by SERS, followed by PDT treatment. In an exemplary embodiment, the nanoprobe system can comprise a silver-embedded gold nanostar that acts as a SERS tag for Raman imaging Photosensitizer molecules can be loaded onto the SERS tag by encapsulating them in a silica shell for PDT treatment. Selective detection and treatment of folate receptor positive cells have been demonstrated when breast cancer cells were used as a folate receptor negative control.

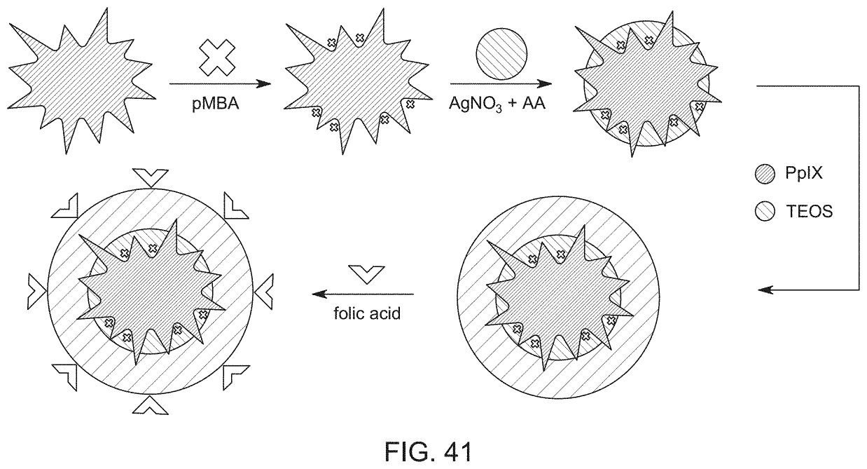

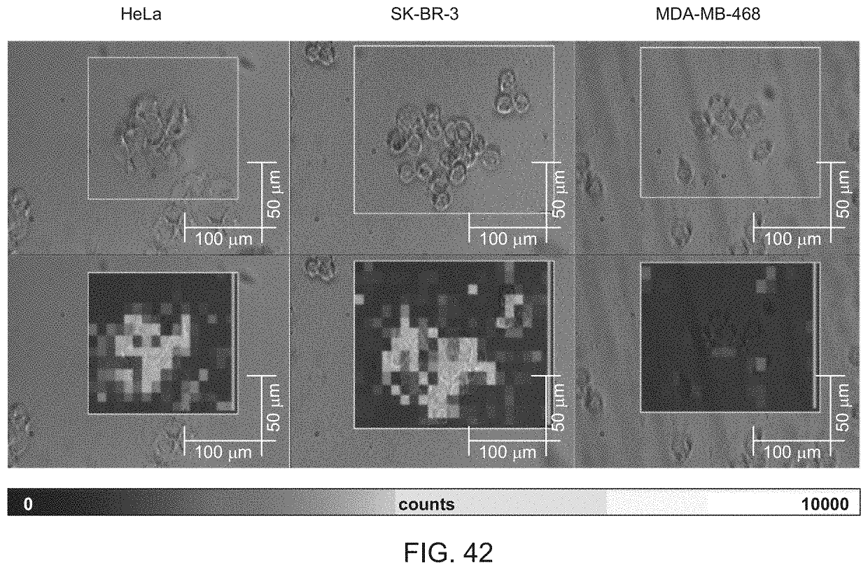

Folate-targeted Raman tags can be synthesized by coating pMBA-labeled nanostars with silver to enhance the SERS signal and subsequently coated with silica using a modified Stöber method. The silica-coated particles can be first modified with (3-aminopropyl)triethoxysilane (APTES) to provide free amine groups on the particle surface prior to being functionalized with Folic acid (FA)-PEG-NHS. FIG. 41 provides a schematic diagram of the synthesis. As shown, gold nanostars are first labeled with the Raman dye pMBA. Silver coating is performed by reducing silver nitrate with ascorbic acid, embedding the pMBA between the gold core and silver shell. The photosensitizer, PpIX, is loaded onto the particles by encapsulation within a silica shell, achieved by adding PpIX during condensation reaction of the silica precursor, TEOS. Targeting functionality is realized by the conjugation of folic acid to the outer surface of the silica shell. FIG. 42 shows Raman/SERS mapping of the three different cell lines after 4 hr incubation with the FA-targeted theranostic nanoprobes at 0.1 nM concentration. The folate receptor positive cell lines (HeLa and SK-BR-3) show high Raman intensity coming from the cluster of cells, while the folate receptor negative cell line (MDA-MB-468) shows little to no Raman signal. Scale bars are 100×50 μm.

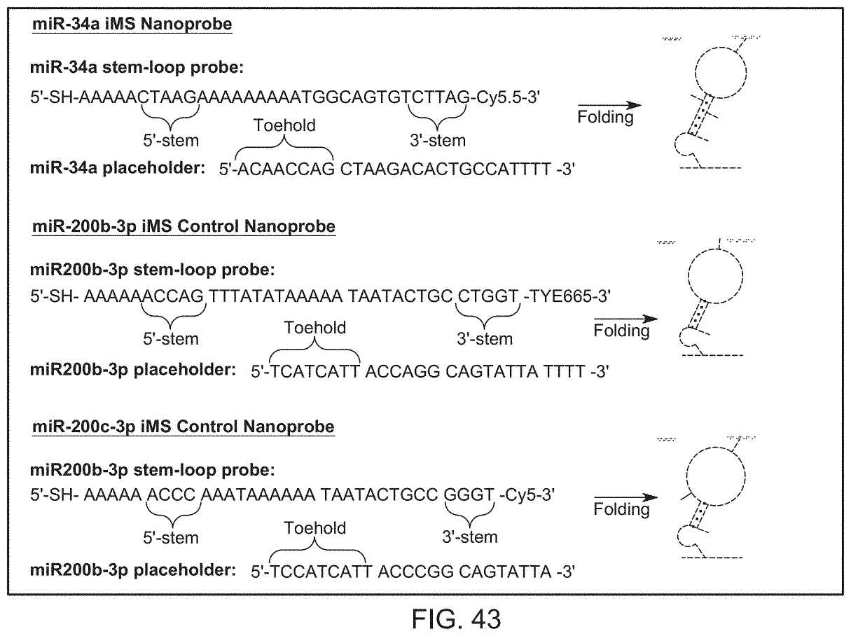

Exemplary embodiments of molecular systems for targeting various miRNA biotargets are shown in FIG. 43. The examples include a miR-34a iMS nanoprobe system, a miR-200b-3p iMS nanoprobe system, and a miR-200c-3p iMS nanoprobe system. The systems include a 5′ stem and a 3′ stem in the oligonucleotide capable of forming a stem-loop configuration and a toehold section in the placeholder segment.

When nanoprobe systems comprising oligonucleotides are used in cell culture or in vivo applications, degradation by nucleases is a concern. Unmodified DNA and RNA oligonucleotides are quickly digested in vitro and in vivo by endogenous nucleases. Multiple endo- and exonucleases exist in vivo. Generally, in serum, the bulk of biologically significant nucleolytic activity occurs as 3′ exonuclease activity, while within the cell, nucleolytic activity is affected by both 5′ and 3′ exonucleases.

To limit nuclease sensitivity, different modifications to the oligonucleotide, including a phosphorothioate bond, can be used. The phosphorothioate (PS) bond substitutes a sulfur atom for non-bridging oxygen in the phosphate backbone of an oligonucleotide. Approximately 50% of the time (due to the 2 resulting stereoisomers that can form), PS modification renders the internucleotide linkage more resistant to nuclease degradation. In an exemplary embodiment of the nanoprobe system, at least 3 PS bond modifications at the 5′ and 3′ oligonucleotide ends can be included to inhibit exonuclease degradation. Including PS bonds throughout the entire oligonucleotide can help reduce attack by endonucleases as well, but may also increase toxicity.

Additional modifications to the oligonucleotide can be made. A naturally occurring post-transcriptional modification of RNA, 2′OMe is found in tRNA and other small RNAs. Oligonucleotides can be directly synthesized to contain 2′OMe. This modification prevents attack by single-stranded endonucleases, but not exonuclease digestion. Therefore, the oligos having 2′OMe modification can also have end block modifications as well. DNA oligonucleotides that include this modification are typically 5- to 10-fold less susceptible to DNases than unmodified DNA. The 2′OMe modification increases stability and binding affinity to target transcripts.

Locked nucleic acids (LNA) are conformationally restricted nucleic acid analogues, in which the ribose ring is locked into a rigid C3′-endo (or Northern-type) conformation by a simple 2′-O, 4′-C methylene bridge. LNA has many attractive properties, such as high binding affinity, excellent base mismatch discrimination capability, and decreased susceptibility to nuclease digestion.

Duplexes involving LNA (hybridized to either DNA or RNA) display a large increase in melting temperatures ranging from +3.0° C. to +9.6° C. per LNA modification, compared to corresponding unmodified reference duplexes. Furthermore, LNA oligonucleotides can be synthesized using conventional phosphoramidite chemistry, allowing automated synthesis of both fully modified LNA and chimeric oligonucleotides such as DNA/LNA and LNA/RNA. Other advantages of LNA include its close structural resemblance to native nucleic acids, which leads to very good solubility in physiological conditions and easy handling. In addition, LNA is nontoxic. All these properties are highly advantageous for a molecular tool for diagnostic applications. LNA can be used in oligonucleotide-based therapeutics.

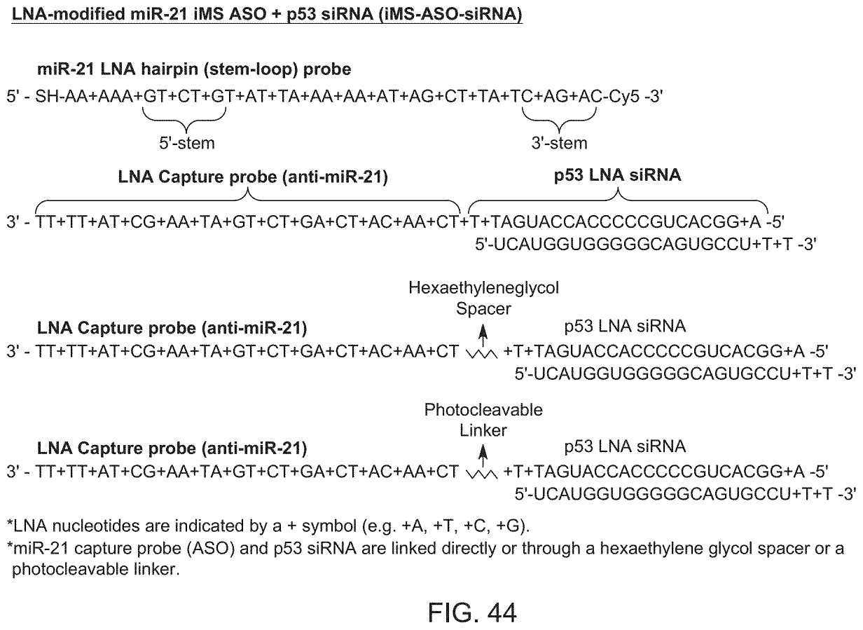

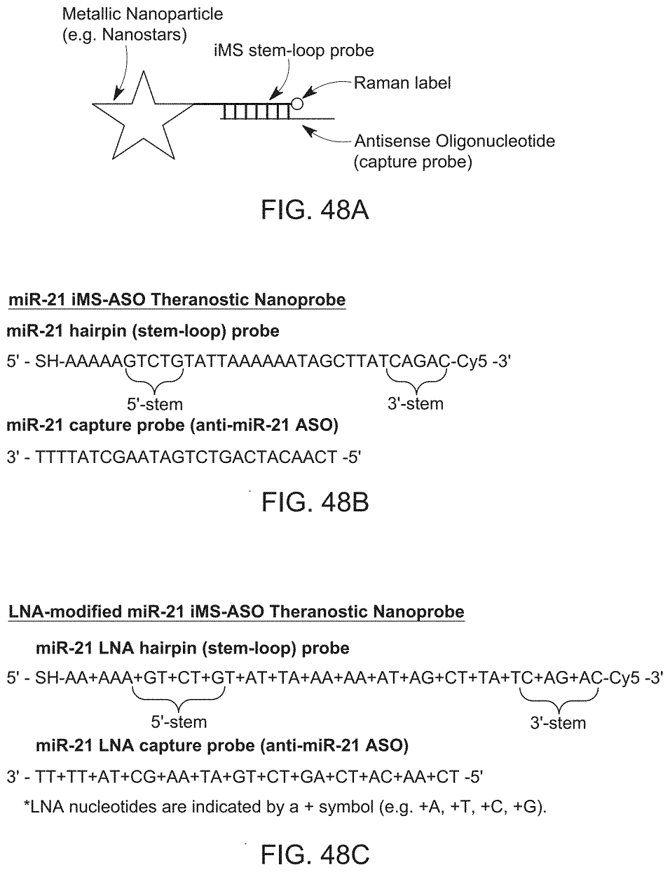

In an exemplary embodiment, a DNA/locked nucleic acid (LNA) chimeric miR-21 iMS and p53-siRNA can be used. FIG. 48c (explained below) shows an iMS-ASO with a LNA, and FIG. 44 shows an iMS-ASO-siRNA with LNAs. LNAs provide high binding affinity, excellent base mismatch discrimination capability, and decreased susceptibility to nuclease digestion.

Thiol linkers can also be used to improve stability of the nanoprobe system. As described above, the oligonucleotide capable of forming a stem-loop configuration (stem-loop probe) is immobilized onto a metallic nanoparticle or nanostar with a metal-thiol bond. The stem-loop probe can be functionalized with either monothiol (such as alkyl-thiol) or multi-thiol anchoring groups (such as one, two or three cyclic disulfide linkers (DTPA) resulting in 2, 4, or 6 metal-sulfur bonds) for binding to a nanoparticle. Multiple thiol groups can increase the binding affinity of oligonucleotides for the metallic surface of the nanoparticle, thus leading to higher stability of metallic nanoparticle-oligonucleotide conjugates.

Raman spectroscopy offers distinct features that are important for in vivo monitoring of cellular systems for a wide variety of applications including drug discovery, biotechnology monitoring, and regenerative medicine. Following laser irradiation of a sample, the observed Raman shifts are equivalent to the energy changes involved in molecular transitions of the scattering species and are therefore characteristic of it. These observed Raman shifts, which correspond to vibrational transitions of the scattering molecule, exhibit very narrow linewidths (one Angstrom or less). For these reasons, Raman spectroscopy has a great potential for multiplexing application. That is, many organic compounds with distinct Raman spectra may be used as dyes to label biological macromolecules and each labeled molecular species will be able to be distinguished on the basis of its unique Raman spectra. This is not the case with fluorescence, because the broad spectral characteristics of fluorescence excitation and emission spectra result in large spectral overlaps if more than 3-4 fluorescent dyes are to be detected simultaneously. Note that the fluorescence emission lines of quantum dots (1-5 nm) are much larger than the Raman peaks (<0.1 nm).

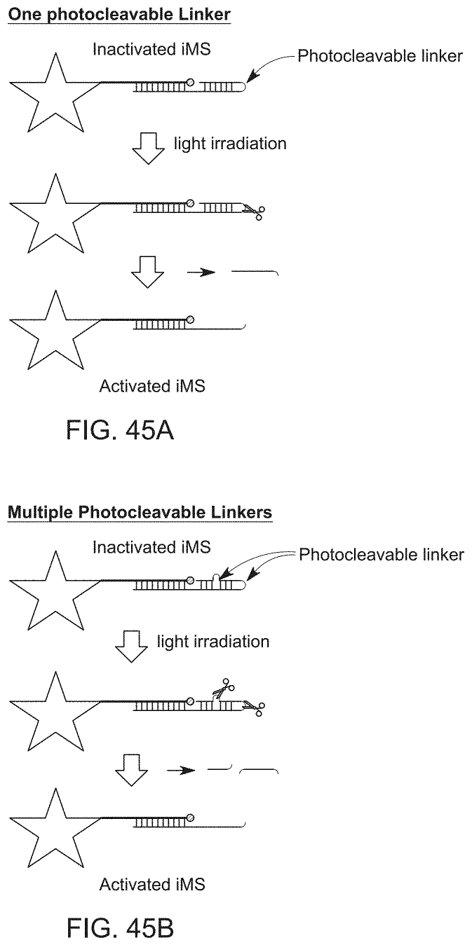

In some applications, it is desirable to keep nanoprobes inactivated (i.e., not operational) for an amount of time before they are activated on demand. The iMS nanoprobe systems described herein can be designed to be photo-activatable biosensors. The nanoprobe systems can be modified to add photocleavable linkers or photo-cages to enable photoactivation. For example, a photocleavable linker can be added within the oligonucleotide backbone. FIGS. 45A and 45B provide schematic diagrams of photocleavable linkers. FIG. 45(A) illustrates a single photocleavable linker, and FIG. 45(B) illustrates multiple photocleavable linkers within the oligonucleotide backbone. As shown, an inhibitor strand complementary to the toehold of a placeholder strand can be tethered to the placeholder strand through a photocleavable linker or linkers.

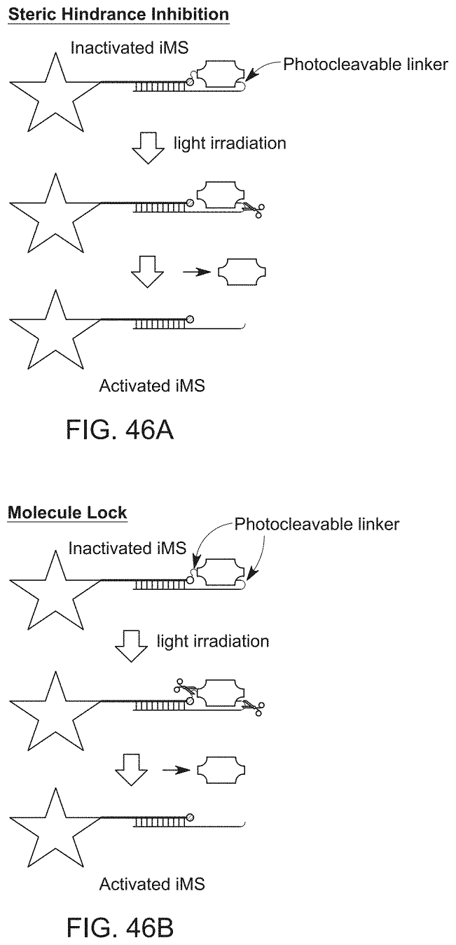

In the absence of light irradiation, the nanoprobe system is inactive. After light irradiation, the linker is cleaved and released, which activates iMS nanoprobe system. In addition, photo-removable macromolecules (e.g. proteins) can be used to inactivate iMS through steric hindrance. FIGS. 46A and 46B provide schematic diagrams of photo-removable macromolecules or a “molecular lock” mechanism

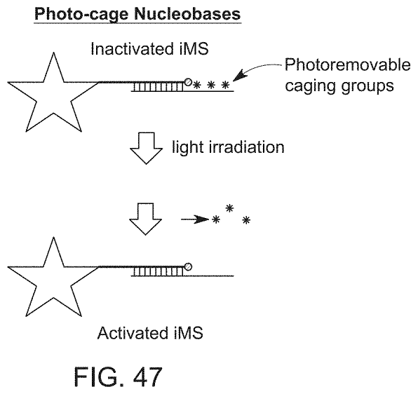

Additionally, photo-cage nucleobases can be used to inactivate iMS by preventing Watson—Crick base pairs between placeholder and target strands. Exemplary photo-removable caging groups, include 1-(ortho-nitrophenyl)ethyl (NPE) and 2-(ortho-nitrophenyl)propyl (NPP). FIG. 47 is a schematic diagram of photo-activatable iMS nanoprobes using photo-removable caging groups to inactivate iMS.

In embodiments wherein the nanoprobe system includes therapeutic components, the placeholder strand of the nanoprobe system can perform as an antisense oligonucleotide (iMS-ASO) and can also have a siRNA linked thereto (iMS-ASO-siRNA). FIG. 48(a) is a diagram showing an exemplary design of an iMS theranostic nanoprobe (iMS-ASO) with the capture probe acting as an antisense oligonucleotide (ASO). FIG. 48(b) is a diagram showing an exemplary design of an iMS-ASO for miR-21 using unmodified oligonucleotides. FIG. 48(c) is a diagram showing an exemplary design of an iMS-ASO for miR-21 using LNA-modified oligonucleotides. The placeholder strand (acting as the antisense oligonucleotide) can target either DNA or RNA in the nucleus or cytoplasm, such as genomic DNA, pre-mRNA, mRNA, microRNA (miRNA), as well as various non-coding RNAs (ncRNAs). For example, the placeholder strand can be used as the anti-miRNA (i.e. miRNA inhibitor) in an iMS-ASO theranostic nanoprobe for antisense-based therapeutics by suppressing the growth of cancer cells with increased apoptosis and decreased cell proliferation.

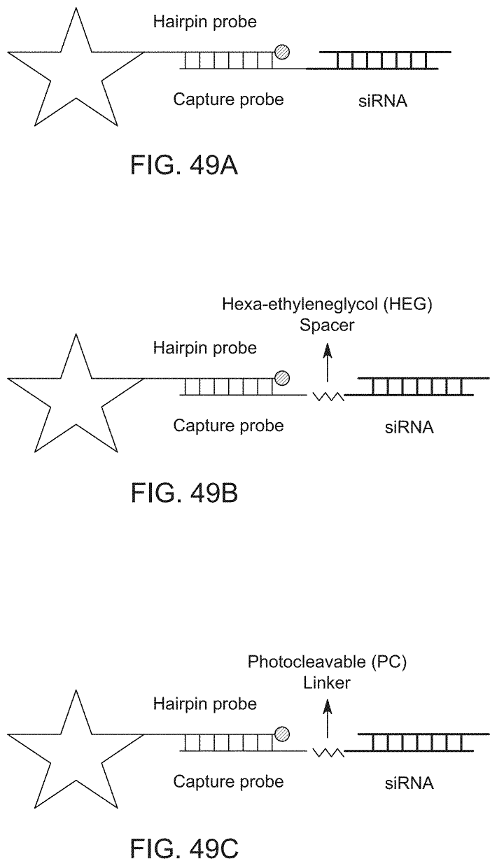

FIGS. 49A, 49B, 49C are diagrams showing an exemplary design of a dual targeting theranostic nanoprobe containing an iMS nanoprobe and siRNA with the capture probe acting as an antisense oligonucleotide (iMS-ASO-siRNA). In FIG. 49(a), the capture probe (ASO) is linked to siRNA directly. In FIG. 49(b), the capture probe (ASO) is linked to siRNA through a hexa-ethylene glycol (HEG) Spacer. In FIG. 49(c), the capture probe (ASO) is linked to siRNA through a photocleavable linker.

In the dual-targeting scheme using the iMS-ASO-siRNA theranostic nanoprobe, the siRNA can be linked to the capture probe (acting as an ASO) directly or through a hexaethyleneglycol spacer. This design is based on the fact that siRNAs can be released from gold nanoparticles by Dicer cleavage. Alternatively, the placeholder can be linked to the siRNA through a photocleavable linker in order to release siRNAs with exposure to UV light in the 300-350 nm spectral range.

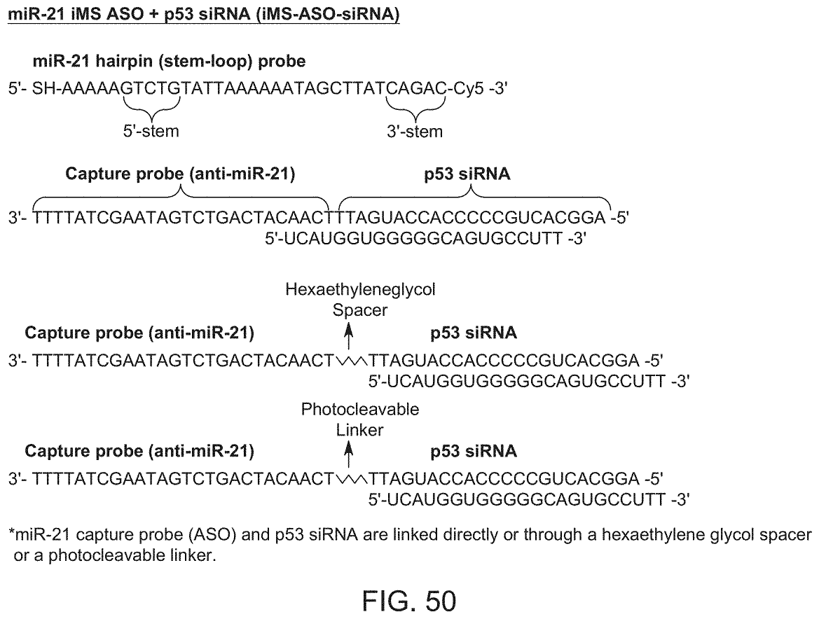

FIG. 50 shows an exemplary embodiment of a dual targeting system with a miR-21 iMS and a siRNA against p53, a common mutated gene in various diseases. Tumor growth has been retarded significantly using p53-specific siRNAs. The capture probe acting as an ASO can be linked with the siRNA directly or through either a hexaethyleneglycol spacer or a photocleavable linker. The capture probe can be designed as a miR-21 ASO (anti-miR-21) and the siRNA can be designed to target R175H p53 mutant.

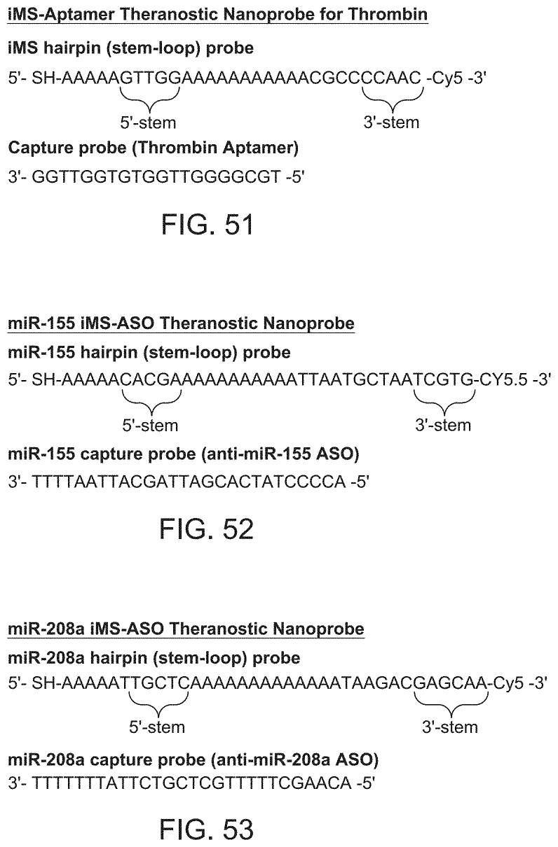

Aptamers can also be used in nanoprobe systems for theranostic applications. For example, thrombin aptamers, which can be used as anticoagulants can be used in the nanoprobe systems described herein. FIG. 51 is a schematic diagram showing an exemplary embodiment of an iMS-Aptamer Theranostic Nanoprobe for Thrombin.

One of the challenges in using nucleic acids as therapeutics is the potential stimulation of an immune response in the subject. For example, depending on the siRNA structure, sequence, and delivery method, siRNAs could stimulate innate immunity, leading to undesired side effects in vivo.

Strategies are available to overcome this challenge. For example, low immunostimulatory vehicles can be used for siRNA delivery. For instance, the innate immune response to densely functionalized, oligonucleotide-modified gold nanoparticles is less in comparison to conventional nucleic acid transfection materials, such as lipoplex. Thus, to avoid a potential immune response, the oligonucleotide density on the iMS-based theranostic nanoprobes can be increased and optimized as a low immunostimulatory system.

In addition, non-immunostimulatory iMS oligonucleotides can be designed using various 2′-modified nucleotides, including but not limited to DNA bases, 2′-O-methyl purines, 2′-fluoropyrimidines, PS linkage modifications, and terminal inverted-dT bases at certain points, which prevent siRNA immune activation. Moreover, immune response increases with the increase in hydrophobicity of the gold nanoparticle surface. Thus, the hydrophobicity of the iMS-based theranostic nanoprobe system can be optimized with PEG linkers having different functional groups.

The nanoprobe systems can be designed to identify and modify different and specific targets for various diseases. For example, an embodiment of a nanoprobe system can be designed to treat brain diseases. As described above, a nanoprobe can be designed to detect and modify miR-21 for cancer therapy. Additionally, a nanoprobe system can be designed against miR-155, which is a key regulator and therapeutic target of many neuroinflammatory and neurodegenerative disorders including Parkinson's disease, Alzheimer's disease, multiple sclerosis, amyotrophic lateral sclerosis (ALS), traumatic brain injury, and autism. FIG. 52 is a schematic illustration of an exemplary nanoprobe system against miR-155. Inhibition of miR-155 with antisense oligonucleotides can upregulate miR-155 expression and extend survival by 38% in an ALS mouse model. Thus, the described nanoprobe system against miR-155 can be effective in treating brain diseases.

Additionally, a nanoprobe system can be designed against miR-208a. miR-208a is a therapeutic target found at much higher levels during cardiac tissue injury in cardiovascular disease. FIG. 53 is a schematic illustration of an exemplary nanoprobe system against miR-208a. Cardiac function, overall health and survival has been shown to be improved by systemically delivered anti-miR-208a during hypertension induced heart failure in Dahl hypertensive rats.

In embodiments, a nanoprobe system can be designed to detect and/or capture circulating tumor DNA (ctDNA) and cell-free DNA (cfDNA), which are nucleic acid fragments that enter the bloodstream during apoptosis or necrosis of normal and diseased cells.

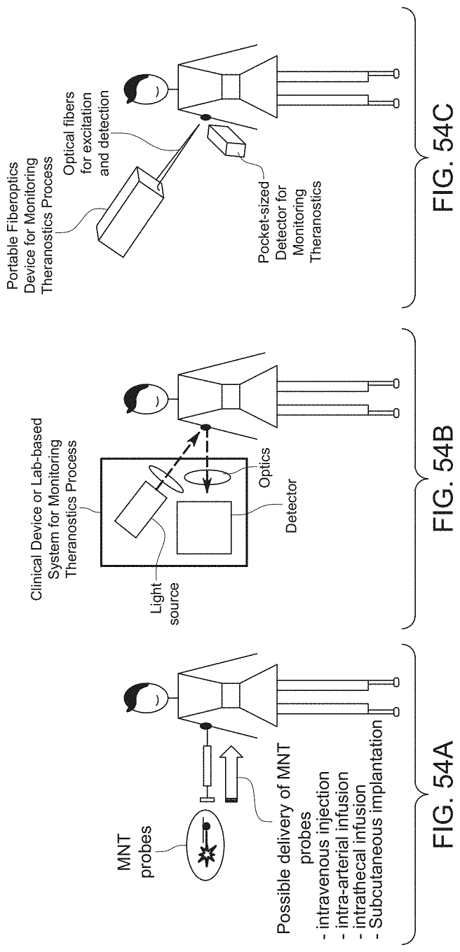

In use, embodiments of the nanoprobe system as described herein can be used for in vivo theranostics that can serve as a real time monitor of the theranostics process to diagnose and provide treatment. FIGS. 54A, 54B, 54C are schematic diagrams illustrating embodiments of how the nanoprobe system can be used for theranostics applications. As shown in FIG. 54A, the nanoprobe system can be delivered using multiple administration methods, including, for example intravenous injection, intra-arterial infusion, intrathecal infusion, and subcutaneous implantation. Various theranostics systems are possible, depending on the degree of miniaturization of the components. For example, as shown in FIG. 54B, detection and monitoring can be performed in a lab setting wherein an excitation light source and optical detector are available. Alternatively, as shown in FIG. 54C, a relatively smaller, portable fiberoptics device using optical fibers for excitation and detection can be used outside of a lab setting. Using available technology, the size of a portable, battery-operated Raman diagnostic and therapeutic system can be reduced to a pocket-sized or wristwatch-sized system. In embodiments, smaller-sized systems can be operated remotely by a smart phone or similar device. It is contemplated, that the theranostics system can be further reduced in size to provide a wearable (e.g., matchbox or coin-size) system.

The nanoprobe systems described herein can be used to monitor the viability, operation, and shelf-life for a wide variety of medical applications, including, but not limited to, stem and precursor cells from a wide variety of sources (e.g., embryos, gestational and adult tissues), stem Cells from reprogrammed differentiated cells, insulin-producing pancreatic islets, tissue-engineered heart muscle, functional tissues and organs, tissue-engineered skin, derived from a patient's own cells, tissue-engineered bladder, derived from a patient's own cells, small intestinal submucosa (SIS), used to help the body close hard-to-heal wounds, tissue-engineered products used to induce bone and connective tissue growth, tissue-engineered vascular grafts for heart bypass surgery and cardiovascular disease treatment, and “Made-to-order” organs from 3D molecular/organic 3D printing.

The nanoprobe systems can also be applied to other non-medical applications. Additionally, nanoprobe systems can be useful in a variety of applications based on DNA/RNA/protein detection including (but not limited to), biomedical applications, point-of-care diagnostics, food safety, environmental monitoring, industrial process sensing, quality control applications, biotechnology industrial control, quality control, global health, cancer research, heart disease diagnostics, and homeland defense.

The nanoprobe system also provide enabling technologies for molecular theranostics applications that can improve cellular systems for in situ in vivo sensing (smart tattoos, implanted sensors), regenerative medicine (stem cells) and other biomedical applications. The nanoprobe systems can be used in a variety of applications including, but not limited to, disease treatment, cancer therapy, food safety, biotechnology, global health, and homeland defense.

### EXAMPLES

### Example 1. Delivery of SERS Nanoprobes into Living Cells

Raman spectroscopy and two-dimensional Raman imaging were used to identify and locate nanoprobes via their surface-enhanced Raman scattering (SERS) detection. To study the efficiency of cellular uptake, silver nanoparticles functionalized with three different positive-, negative-, and neutrally-charged Raman labels were co-incubated with cell cultures, and allowed to be taken up via normal cellular processes. The surface charge on the nanoparticles was observed to modulate their uptake efficiency during a four-hour co-incubation, demonstrating a dual function of the surface modifications as tracking labels and as modulators of cell uptake.

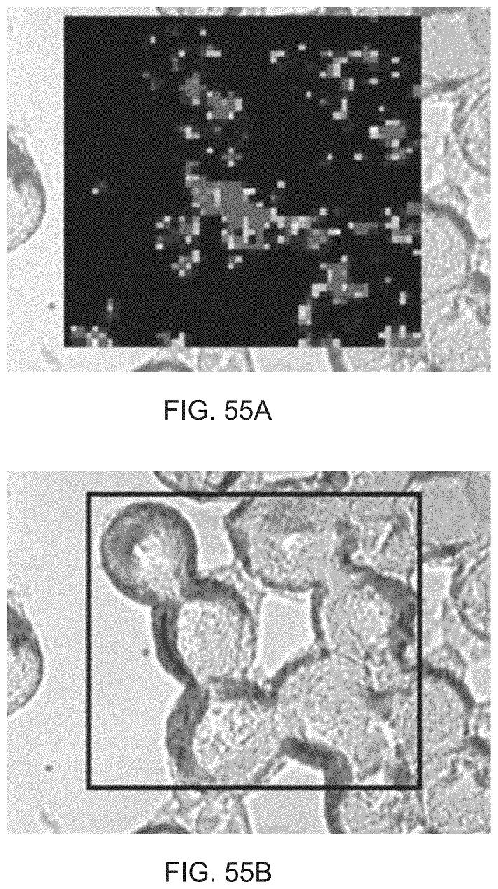

The results indicate that the functionalized nanoprobe construct has the potential for sensing and delivery in single living cells. FIG. 55 is a SERS map showing cellular distribution in J774 cells following incubation for four hours with 4-ATP dye-labeled silver nanoparticles.

### Example 2. Protection of iMS Nanoprobe Systems from Complex Media by Encapsulation in a Gel Matrix

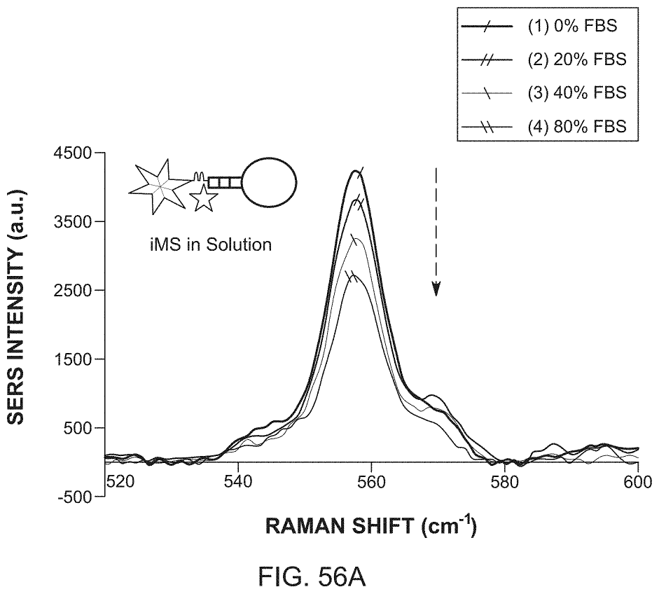

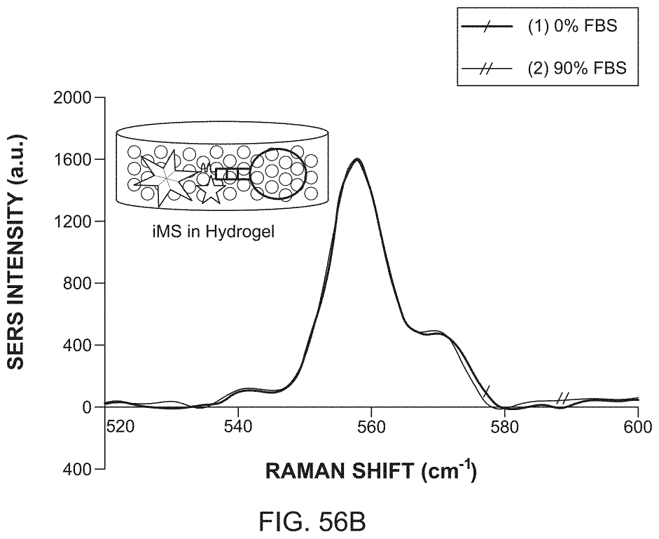

The effect of complex media, such as serum, on the operation of the iMS nanosensor was investigated. Cy5-labeled iMS nanosensors (0.05 nM) were prepared and incubated with 1 μM of synthetic target DNA in a PBS buffer solution containing different concentrations of fetal bovine serum (FBS), (1) 0%, (2) 20%, (3) 40% and (4) 80% FBS, for 1 hour. Following incubation, SERS measurements were performed using a Renishaw InVia confocal Raman microscope equipped with a 632.8-nm HeNe laser. FIG. 56a is a chart showing the results of testing. As can be seen, increasing the concentration of FBS decreases the SERS signal of the Cy5-labeled iMS in the ‘On’ status. At 80% FBS (spectrum d), the SERS signal of the iMS drops to 65% of its original signal intensity (0% FBS, spectrum a), indicating that the nanosensor response was affected by the presence of FBS. To protect the nanosensors, they were embedded in a poly-NIPAM gel matrix and the response of the embedded nanosensors in the complex media was evaluated. The embedded nanosensors were evaluated after 3.5-hr incubation in a PBS buffer solution containing (1) 0% or (2) 90% FBS. FIG. 56b is a chart showing the results of testing. As can be seen, even at 90% FBS (spectrum b) the SERS signal of the iMS (in the ‘On’ status) is similar to that in aqueous solution (spectrum a), illustrating the protection capability of a gel matrix against complex media.

### Example 3. SERS Nanosensors for In Vivo Detection of Nucleic Acid Targets in a Large Animal Model

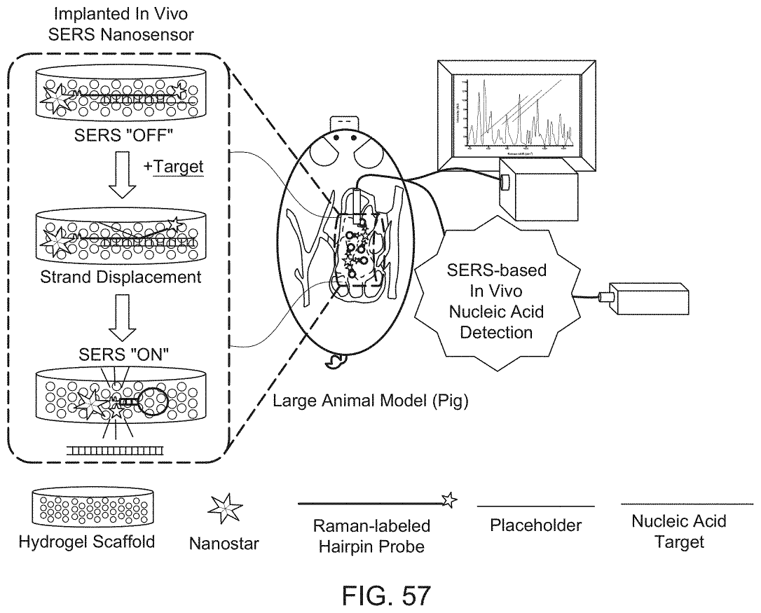

This example provides proof-of-principle of in vivo detection of nucleic acid targets using a SERS nanosensor implanted in the skin of a large animal model (pig). FIG. 57 is a schematic diagram of exemplary SERS nanosensors for in vivo detection of nucleic acid targets in a large animal model. It will be recognized that this test shows proof of concept for other large animals, in addition to pigs, as well. The in vivo nanosensor involves the “inverse Molecular Sentinel” (iMS) detection scheme using plasmonics-active nanostars, which have tunable absorption bands in the near infrared (NIR) region of the ‘tissue optical window’, rendering them an efficient sensing platform for in vivo optical detection.

### Example 4. Toxicity Evaluation of Gold Nanostars

Toxicity evaluation of gold nanostars (GNS) was performed to demonstrate that they are a biocompatible platform for in vivo applications. Most of the GNS nanoparticles were cleared from the blood circulation by macrophages in the spleen and liver when examined at one week after IV injection with a dose of 20 mg/kg. For the 6-month long-term toxicity study, mouse body weight was monitored weekly and there was no statistically significant weight difference by mixed-model ANOVA analysis between the control group and groups receiving GNS doses up to 80 mg/kg. All mice were carefully monitored and did not exhibit stress or any other abnormal behavior. Mice were sacrificed 6 months after GNS injection and plasma was harvested for blood chemistry evaluation that included metabolic function of kidney and liver. One-way ANOVA statistical analyses demonstrated no statistically significant difference between control and treated groups receiving 20 mg/kg or 80 mg/kg GNS. We also performed histopathological examination of H&E stained brain, heart, liver, kidney, spleen and lung 6 months after GNS injection. No findings indicative of GNS-related toxicity were identified in mice receiving a GNS dose up to 80 mg/kg. Test groups were comparable to the control group and no deleterious effects were noted from systemic administration of GNS.

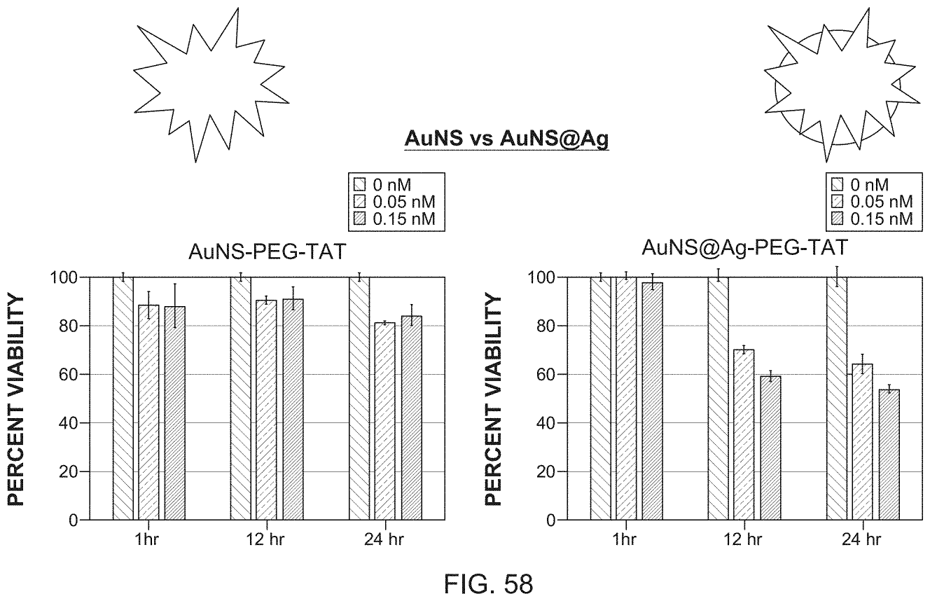

Testing was performed to demonstrate that gold nanostars with silver nanoshells, i.e., AuNS@Ag, exhibit little toxicity and can be used for in vivo studies. FIG. 58 provides charts showing biosafety data for two of different types of GNS: AuNS@Ag-PEG-TAT compared to AuNS-PEG-TAT.

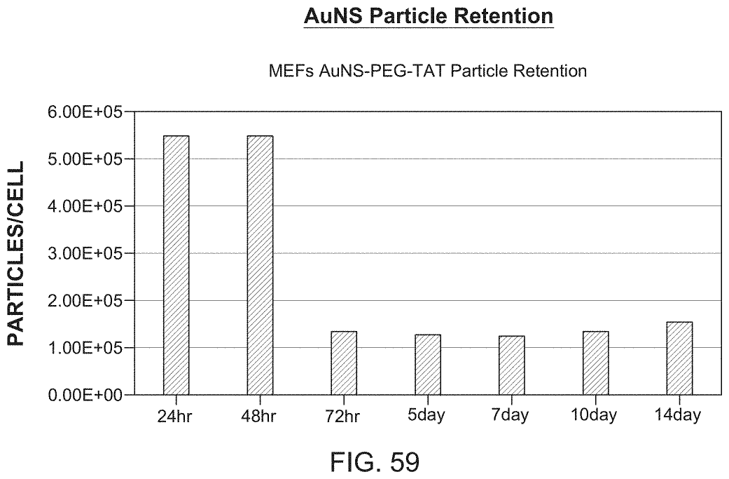

Additionally, a particle retention study was performed using gamma irradiated mouse embryonic fibroblasts. There was a significant loss of particles between 48 and 72 hrs, but thereafter, a steady state was reached. Although many particles were expelled within the first 72 hrs, data showed that cells remained alive for 2 weeks with AuNS inside and retained about 100,000 particles/cell. FIG. 59 is a chart showing results of the GNS particle retention study.

### Example 5. SERS Nanoprobe Systems in Plant Cells

The Tritech Research microINJECTOR was used to inject colloidal nanoprobes into single plant cells through Femtotips microinjection capillary tips (Eppendorf), which have opening diameters of 0.5 μm±0.2 μm. An MES-KCl buffer (100 mM KCl in 5 mM MES, pH 6.5) was used atop plant cells throughout microinjection.

A Renishaw inVia Raman microscope equipped with a 633 nm He—Ne laser, running WiRE 2.0 software, was used to acquire the Raman spectra. Cells were located under brightfield transillumination with a 10× objective. The motorized stage was then set to scan the sample in a grid pattern with 30 pm step size while acquiring a spectrum at each point. For the Cy5 labeled probes, the grating was set to 558 cm-1, and the exposure time was 1 s. The false-color Raman maps were created by integrating the signal to baseline of the 558 cm-1 Raman peak of Cy5 from 533 to 583 cm-1 in the WiRE 2.0 software. For the Cy5.5 labeled probes, the grating was set to 1468 cm-1, and the exposure time was 1 s. The false-color Raman maps were created by integrating the signal to baseline of the 1468 cm-1 Raman peak of Cy5.5 from 1443 to 1493 cm-1 in the WiRE 2.0 software. The color scale between samples was kept the same.

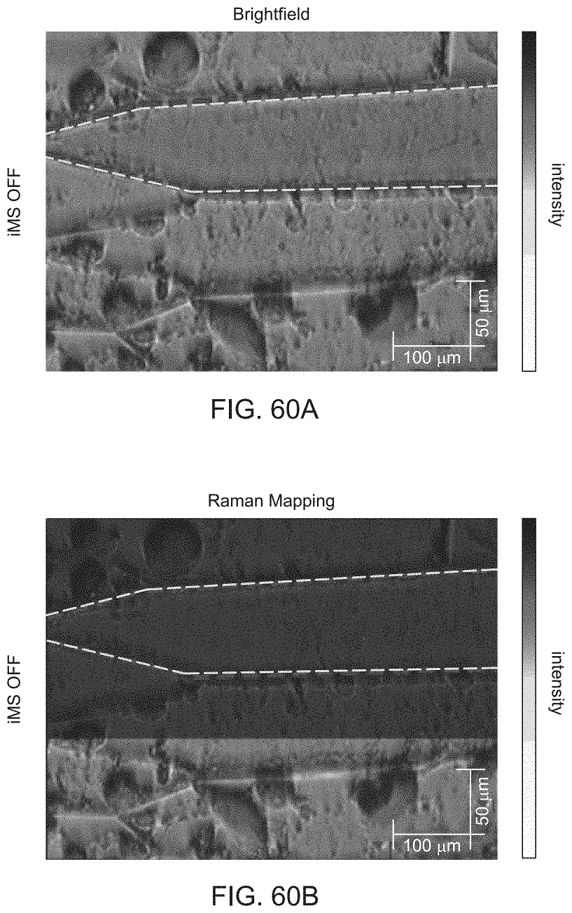

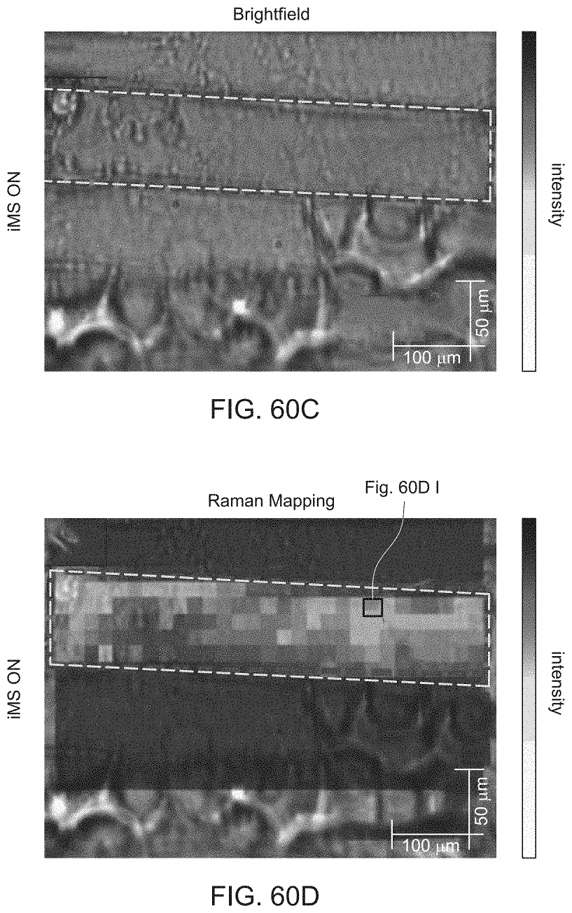

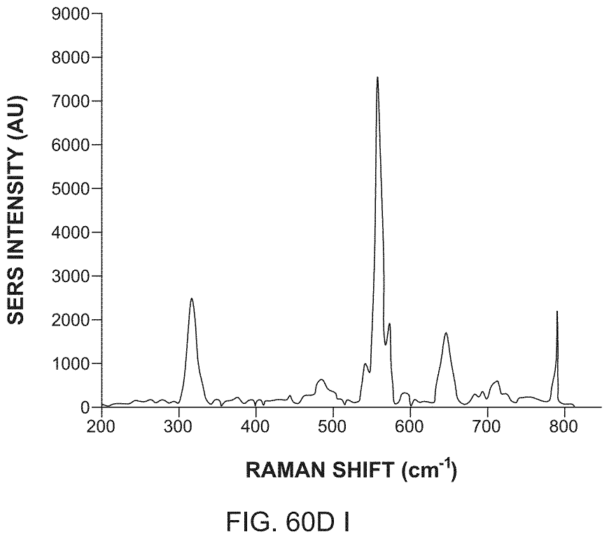

Particle-based nanoprobe injection and detection within single plant cells was first accomplished using onion epidermal cells. The iMS probe specific to microRNA21, which was labeled with the Cy5 dye, was used. The iMS-OFF (probe open) and iMS-ON SERS nanoprobes (probe closed) were injected into single onion cells and Raman mapping was performed. FIG. 60 is an image of Raman mapping of plant cells incubated with iMS nanoprobes.

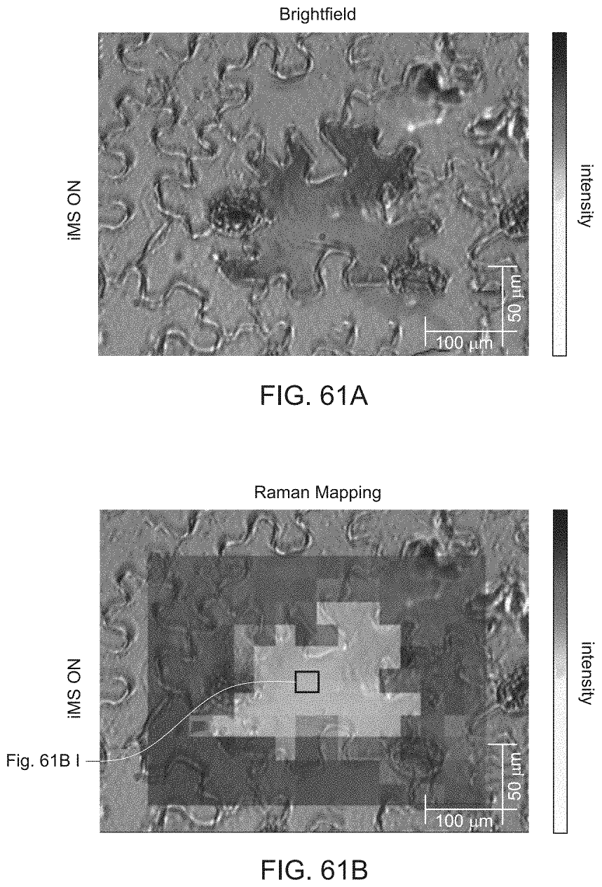

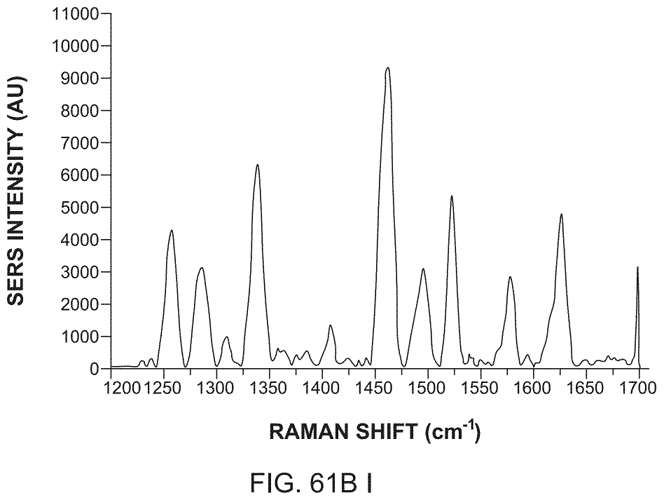

Nanoprobe injection and detection was then demonstrated within single Vicia Faba pigment cells using the microRNA21 iMS SERS nanoprobe. The iMS-ON SERS nanoprobes (probe closed) were injected into pigment cells. FIG. 61 is an image of Raman mapping of plant cells incubated with iMS nanoprobes. As shown in FIG. 61, Raman mapping confirms nanoprobe localization to single cells.

### Example 6. In Vivo Diagnostics Using a Nanoprobe System

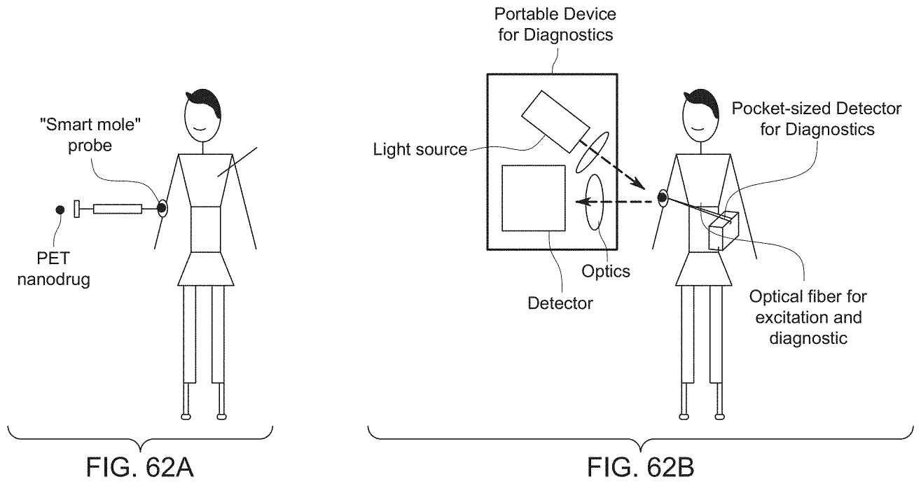

FIGS. 62A and 62B are schematic diagrams showing an exemplary operating procedure using nanoprobe systems for in vivo diagnostics for real time, permanent and continuous ‘health monitor’ of systems (stem cells, tissue-engineered organs, etc.) implanted in a subject (for example, a human subject).

Various diagnostics systems are possible, depending on the degree of miniaturization. For example, detection of a target can use a portable Raman diagnostic system having an excitation light source and an optical detector. FIG. 62B illustrates this concept. An alternative diagnostic system can include a pocket-sized (or palm-sized) battery-operated Raman diagnostics system that is linked to a ‘smart mole’ by fiberoptic excitation and detection. FIG. 62A illustrates this concept. The pocket-sized system can be operated remotely by a smart phone or similar device. Miniaturization can shrink the size of the portable diagnostic system into the size of a ‘wristwatch-sized’ battery-operated Raman diagnostics device. Further miniaturization can provide a wearable (matchbox or coin-size sized) detection system.

### Example 7. iMS Nanoprobes with Locked Nucleic Acids (LNA) for Improved Stability

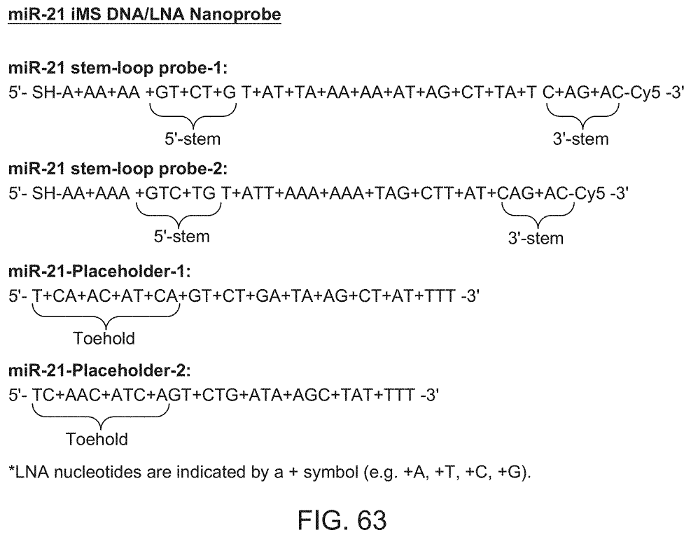

An exemplary embodiment of a DNA/LNA chimeric iMS was designed to allow for a longer lifetime of the nanoprobe system within cells. FIG. 63 is an illustration showing the design of iMS nanoprobes targeted to miR-21. Sequences with DNA/LNA alternating bases or all LNA bases can resist nonspecific protein binding and DNase I digestion. Additionally, in testing, a sequence consisting of a DNA stretch having less than three bases between LNA bases was able to block RNase H function. In FIG. 63, LNA is indicated by +. An oligonucleotide having a five base-pair stem and alternating DNA/LNA bases is effective for intracellular applications as it ensures reasonable hybridization rates, reduces protein binding and resists nuclease degradation for both target and probes.

### Example 8. Multiplexed Detection of miRNAs Using Differently Labeled iMS Nanoprobes

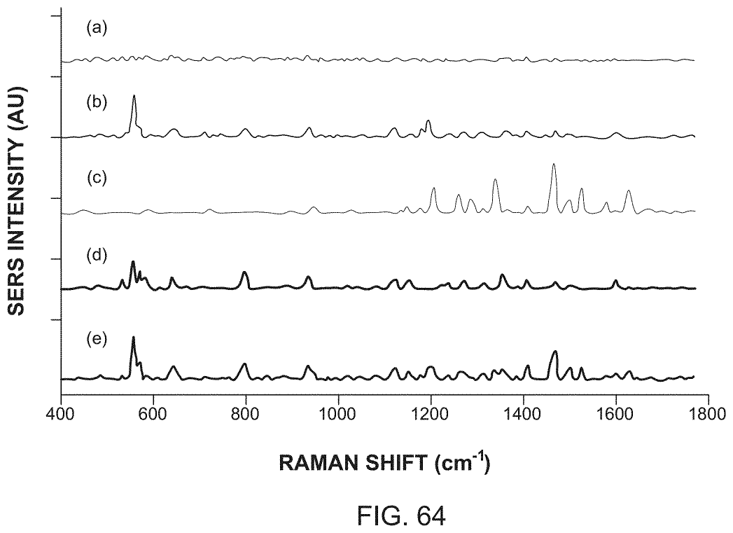

Due to narrow Raman bandwidths, SERS nanoprobe systems have multiplexing capability. Three iMS nanoprobes differently labeled with Cy5, Cy5.5, and TYE665 were tested for the detection of two miRNA biomarkers, namely miR-21 and miR-194, for GI cancer, as well as, a control miRNA, miR-39. FIG. 64 is a chart showing SERS spectra of the three differently labeled iMS nanoprobes targeting to miR-21, miR-194 and miR-39. In FIG. 64, spectra (a) is blank, spectra (b) is in the presence of miR-21 target, spectra (c) is in the presence of miR-194 target, spectra (d) is in the presence of miR-39 target, and spectra (e) is in the presence of a mixture of miR-21, miR-194 and miR-39 targets. FIG. 64 shows that the iMS nanoprobes are capable of simultaneously detecting and discriminating the three miRNA targets. Spectra were baseline corrected and offset for clarity.

The SERS spectra of the three nanoprobes exhibit multiple unique Raman peaks. The SERS sensing modality provides specific spectral “fingerprints” with very sharp peaks allowing sensing multiple targets simultaneously in a single assay platform. The SERS measurements were performed immediately following the incubation of samples without any washing steps to simplify and accelerate the assay procedure.

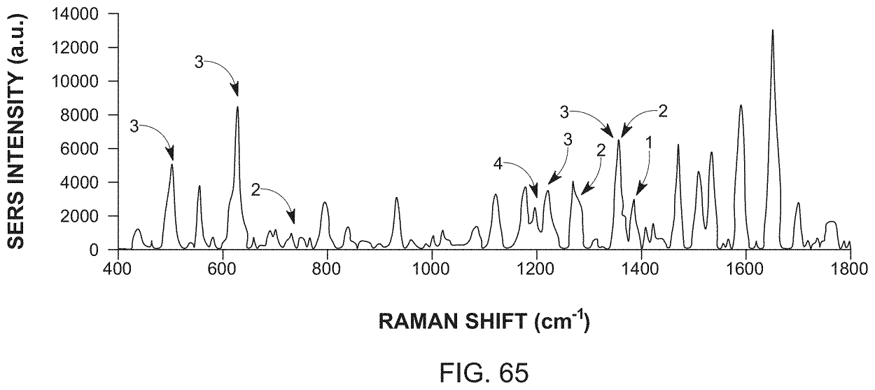

Multiplex detection was also demonstrated by mixing nanoprobes labeled with different dyes, including TYE563, Rhodamine Red, TAMRA, and Cy3. The dyes absorb light around 633 nm, which is required for resonance Raman and strong SERS signal while using a 633-nm laser as the excitation light source. Their distinctly different SERS peaks allow for their simultaneous detection. FIG. 65 shows an example of a mixture of the four distinct dyes measured using 633 nm laser excitation for non-resonant SERS. In FIG. 65, a SERS spectrum of a mixture of four nanoprobes differently labeled with (1) TYE563, (2) Rhodamine Red, (3) TAMRA, and (4) Cy3 is shown. The numbers denote the corresponding unique SERS peaks of the dyes.

### Example 9. Raman/SERS Hyperspectral Imaging (HSI) and Raman Data Cube for Cellular Systems

We can use Raman spectroscopy as a modality for multiplex in vivo monitoring of cellular systems as well as in vitro detection in ultra-high throughput microarray systems. Using different tags for each reaction, it is possible to monitor several reactions simultaneously (label-multiplexing). Fluorescence has been often used as a detection method in microarrays. However, because fluorescence spectra have relatively broad band features, spectral overlap is a limitation for the use of a large number of fluorescence labels simultaneously. Due to narrow absorption bands and large spectral range, Raman provides much greater capabilities for multiplexing than fluorescence. Thus, Raman spectroscopy can be used for detection in ultra-high throughput microarray systems. The SERS technique enables the use of many different probe molecules, allowing the narrow band spectral characteristics of Raman-based probes to be used for sensitive, specific analysis of microarrays.

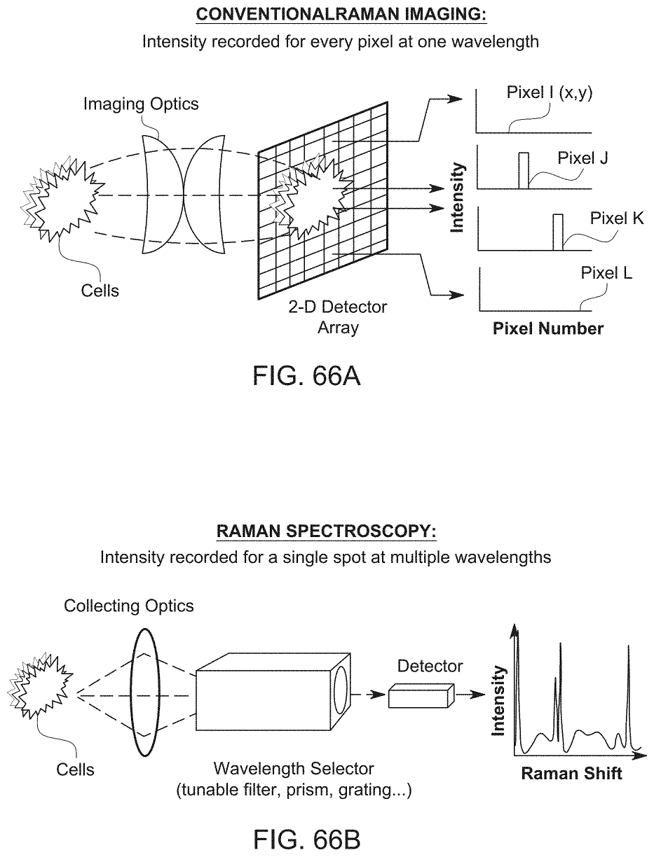

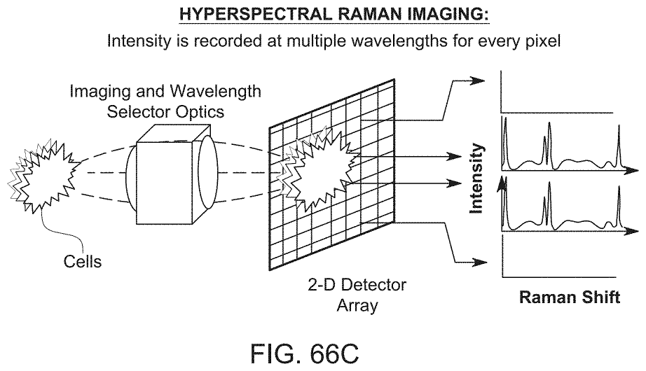

FIG. 66 is a schematic diagram illustrating the basic principle of Raman/SERS Hyperspectral Imaging (HSI). HSI represents a hybrid modality for optical diagnostics, whereby spectroscopic information is collected and rendered into an image. FIG. 66(A) relates to conventional Raman imaging, wherein every pixel of an entire two-dimensional image can be recorded, but only at a specific wavelength or spectral bandpass. FIG. 66(B) illustrates that in conventional Raman spectroscopy, the optical spectrum at every wavelength within a spectral range can be recorded, but for only a single spot (or pixel); and FIG. 66(C) illustrates that the HSI concept combines the two recording modalities and allows recording the entire optical spectrum for every pixel on the entire two-dimensional image in the field of view. HSI combines two recording modalities (spectroscopy and imaging) and allows the recording of the entire scattering spectrum for every pixel on the entire image in the field of view.

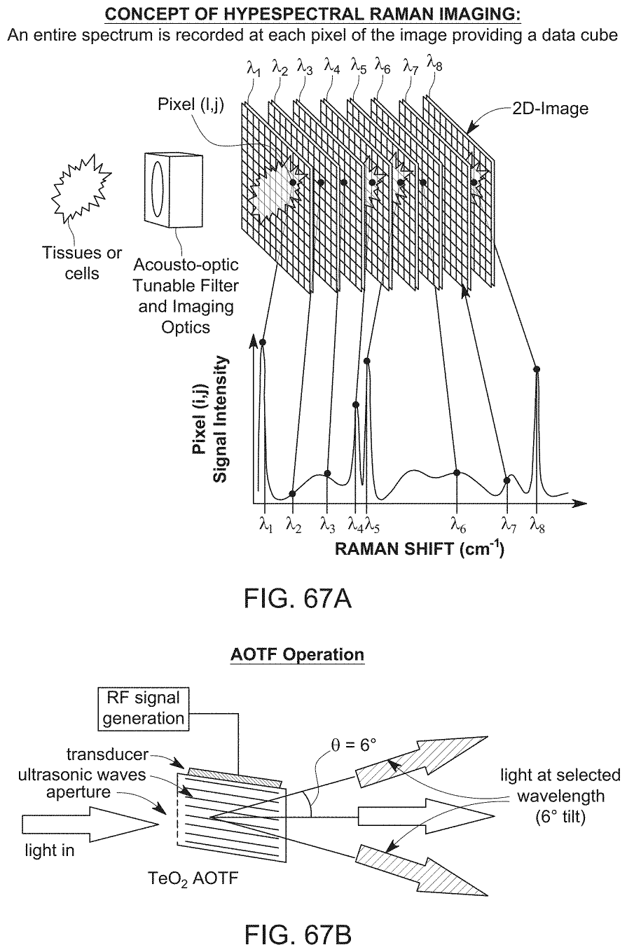

The resulting hyperspectral image may be presented as a 3-D data cube consisting of two spatial dimensions (x, y) defining the image area of interest and the spectral dimension (2) used to identify chemically the material at each pixel in the image. FIG. 67(A) is a schematic diagram illustrating a hyperspectral data cube showing the spectral information of a series of Raman images collected at various wavelength of interest. FIG. 67(B) is a schematic diagram of a noncollinear AOTF device showing the diffraction of unpolarized light into three beams: Two narrow-band diffracted (dark arrows) and a broadband undiffracted beam (white).

Hyperspectral Imaging Instrumentation

The HSI system uses a rapid wavelength-scanning solid-state device, a non-collinear TeO₂ AOTF, which operates as a tunable optical bandpass filter. An AOTF offers the advantage of having no moving parts and high transmission efficiency (as high as 98% at selected wavelengths) that translates into high sensitivity, thus allowing fast data acquisitions. Since AOTFs with high spatial resolution and large optical apertures are commercially available, they can be applied for spectral imaging applications. A confocal SERI system that combines a two-dimensional ICCD, APD detector and an AOTF device for hyperspectral Raman imaging applications is used.

Tunable filters, such as AOTFs and liquid crystal tunable filters (LCTFs), allow the investigator to rapidly record images at various wavelengths. An AOTF is a compact, electronically controlled bandpass filter which operate over a wide wavelength range from the ultraviolet (UV) to the far infrared (IR). The operation of an AOTF is based on the interaction of light (incident light) with an acoustic wave in a birefringent crystal. The device consists of a piezoelectric transducer bonded to one side of the crystal. The transducer emits vibrations (acoustic waves) when a radio frequency (RF) is applied to it. When an acoustic wave is generated in the crystal, a periodic modulation of the index of refraction of the crystal is established via the elasto-optic effect. This then creates a grating by alternately compressing and relaxing the lattice. Unlike a classical diffraction grating, AOTF only diffract one specific wavelength of light and as a result act as a tunable filter.

The wavelength of the diffracted beam is varied by changing the RF signal applied to the crystal, thereby adjusting the grating spacing. The Bragg grating diffracts only light that enters the crystal within an angle normal to the face of the crystal. This range is called the acceptance angle of the AOTF while the percentage of light diffracted is the diffraction efficiency of the device. The latter parameter greatly depends on the incidence angle, the wavelength selected and the power of the RF signal. Unlike commercial spectrometers where the bandwidth is fixed using interference filters incorporated into filter wheels, AOTF and LCTFs can rapidly vary (typically in less than 50 μs) the bandwidth by using closely spaced rf signals simultaneously. AOTFs can either be collinear (the incident and the diffracted light and the acoustic wave travel in the same direction) or non-collinear (the incident and the diffracted light beams travel in different directions from the acoustic wave. A non-collinear AOTF separates the first-order beam from the undiffracted beam (zero-order beam). The undiffracted light exits the crystal at the same angle as the incident light while the diffracted beam exits the AOTF at a small angle (˜6°) with respect to the incident light. The principle of operation of a non-collinear AOTF is schematically illustrated in FIG. 67B.

Instrumentation

A confocal hyperspectral Raman imaging system was used. The system consists of a Nikon Diaphot 300 Inverted microscope (Nikon, Melville, N.Y.) coupled with a 15 mW HeNe Laser (Melles Griot, 05-LHR-171) operating at 632.8 nm as the SERS excitation source. The light from the laser was passed through a set of diverging and collimating lenses (L1), an iris, and then diverted into a microscope objective (60×, 0.85 NA) using a dichroic filter (Omega Optical, 630DRLP,) and focused on a sample mounted onto the X-Y-Z translation stage. SERS signals were collected by the same objective, transmitted through the dichroic mirror, and then through a holographic notch filter (HNF) (Kaiser Optical System, 633 nm) into the AOTF device. The AOTF (Brimrose, TEAFS-0.6-0.9-UH) projected the diffracted (first-order) light at an angle different from the undiffracted (zero-order) light. The AOTF has a spectral operating range from 600-900 nm which corresponds to the relative wavenumber range (from 0 to 4691.7 cm⁻¹ with respect to a 632.8 nm excitation and a spectral resolution of 7.5 cm⁻¹ at 633 nm. The first order beam exiting the AOTF was passed through a beamsplitter (BS) (70/30 ratio), then through a second Iris and imaged onto a thermoelectrically cooled intensified charged-coupled device (ICCD) containing a front-illuminated chip with 512×512 two-dimensional array of 19×19 um² (PI-Max:512 GEN II, Roper Scientific, Trenton N.J.). The ICCD was computer controlled with WinView software. The reflected beam (30%) was focused down onto the active area of an avalanche photodiode (APD) (SPCM-AQR-14, Perkin Elmer). An APD is an ideal detector for several reasons: small size, high quantum efficiency (QE) and large amplification capabilities. The APD used has a QE of ˜70% and very low dark count (<100 c/s) thus reducing the possible noise arising from the use of amplifiers. A TTL pulse of 2.5 volts is sent to a universal counter where the pulses are counted for a specified acquisition time. The APD detector is controlled by an integrated LabVIEW program developed in house. The AOTF based HSERI system is also integrated with incandescent tungsten light for bright field imaging and a mercury lamp for fluorescence imaging. In addition the SERS excitation source and the optics can be easily changed to suite any other application requiring an alternative excitation. SERS spectra and images were acquired after focusing the laser beam to an area of interest on sample or cells adhered to the glass chamber slides. The images were acquired upon excitation with 15-mW laser power and an accumulation time of 6 s (0.6 s/frame). The SERS spectra were recorded with accumulation times of 25-50 s.

### Example 10. Design of iMS Nanoprobe Systems Using Poly(A) Spacers to Decrease Background

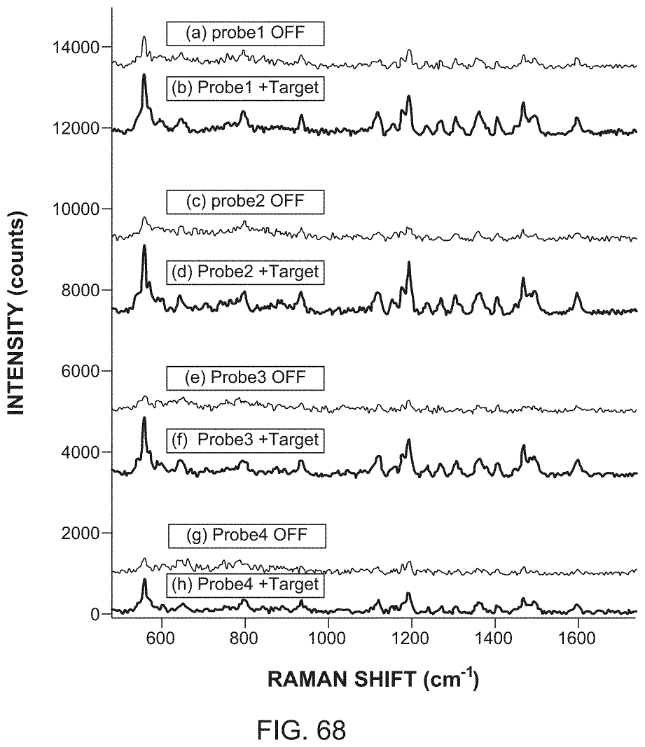

For in-vivo sensing in this example, gold nanostars (AuNS) were used in the nanoprobe system. To examine the functionality of iMS nanoprobe system with AuNS, four different probes were tested with their corresponding synthetic targets. Probe-1 contained both an internal poly(adenine) spacer and a 5′-end spacer with 5 adenine bases between the hairpin probe and the AuNS surface. Probe-2 and probe-3 only contained the 5′-end spacer. Probe-4 contained neither the internal spacer nor the 5′-end spacer. The results showed that all probes can be turned ON in the presence of their specific targets. However, probe-1, with the internal poly(adenine) spacer had higher background signal than the other 3 probes when the probes were OFF. FIG. 68 is a chart showing SERS spectra for the exemplary probes. Adenine-Au interactions are stronger than cytosine-Au, guanine-Au and thymine-Au interactions, thus, the poly(adenine) spacer interacted with the AuNS surface and affected the hybridization between the oligonucleotide (hairpin probes) and the placeholder strands, leading to a high background signal.

### Example 11. Gold Nanostars as Position Sensors and Trackers of Adipose Stem Cells Using Two-Photon Luminescence (TPL)

Surfactant-free gold nanostars (AuNS) having multiple sharp branches and a plasmon tunable in the near-infrared ‘tissue optical window’ can be used in biomedical applications. The multiple sharp tips create a “lightning rod” effect that further enhances the local surface plasmon. The combined effect from the two properties brings forth high SERS intensity and enhanced two-photon photoluminescence (TPL). The AuNS have a two-photon action cross section (TPACS) up to 10⁶-10⁷ Goeppert-Mayer units (GM), which is higher than that of quantum dots (QDs; 10⁴-10⁵ GM) and organic fluorophores (10²-10³ GM). The AuNS can be used to study particle uptake mechanism in vitro and real-time particle tracking in vivo. In addition, high extinction coefficient (10⁹-10¹⁰ M⁻¹ cm⁻¹) in the NIR region allows AuNS to be used as an exogenous contrast for in vivo photoacoustic mapping. The TPL method allows the use of NIR light for deep tissue excitation into the “optical window” of tissue.

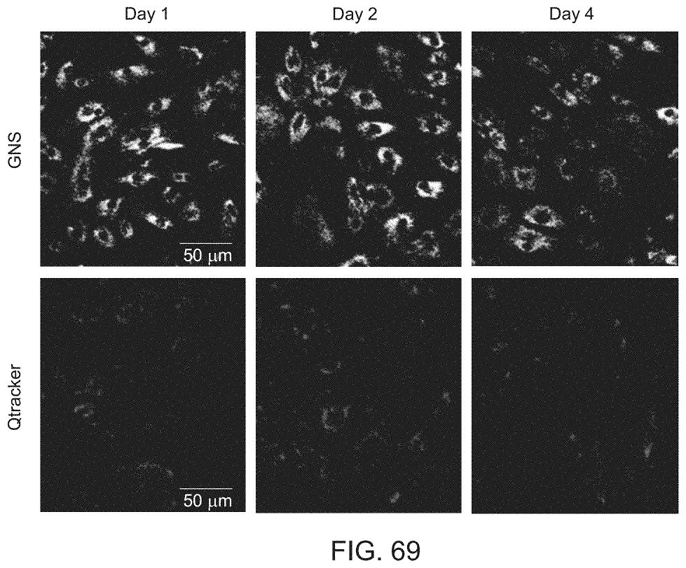

AuNS were used to track adipose-derived stem cells (ASCs). To test the labeling efficiency of AuNS, undifferentiated ASCs were incubated for 24 hours with 0.14 nM of either Qtracker or AuNS that were functionalized with PEG and the HIV-1 protein-derived TAT peptide to promote uptake. Undifferentiated adipose-derived stem cells were stained with Hoechst33342 after a 24-hour incubation with 0.14-nM gold nanostars, and imaged with multiphoton microscopy. Gold nanostars localized to the cytoplasm of ASCs. Furthermore, cells were fixed on days 1, 2, or 4 after incubation with particles or QTracker, and imaged using two-photon photoluminescence (TPL) microscopy (FIG. 69). FIG. 69 provides a series of images enabling comparison of two-photon photoluminescence in gold nanostar- versus Qtracker-labeled adipose-derived stem cells. Undifferentiated adipose-derived stem cells are shown after a 24-hour incubation with 0.14-nM gold nanostars (above) and 0.14-nM Qtracker (below) (scale bars=50 pm). Cells were fixed on days 1, 2, and 4 and then imaged to determine the relative fluorescence of each optical label. Each cell could be roughly distinguished by its predominant fluorescence. Qtracker (red) and gold nanostars (white) could be seen in each cell. Fluorescence was measured over a period of 4 days. Calculated total cell fluorescence was calculated with ImageJ and analyzed using a two-way analysis of variance. There was a significantly greater degree of fluorescence emitted by those cells labeled with gold nanostars versus Qtracker throughout all 4 days of cellular proliferation (p<0.0001). Multiphoton microscopic images were taken at 800 nm using a laser power of 3.7 mW.

The effects of AuNS on cell phenotype, proliferation, and viability was assessed with flow cytometry, trypan blue, and MTT assays (3-(4,5-dimethylthiazol-2-yl)-2,5-diphenyltetrazolium bromide) respectively. Over 4 days, AuNS exhibited stronger TPL than Qtracker and did not affect cell phenotype, viability, or proliferation.

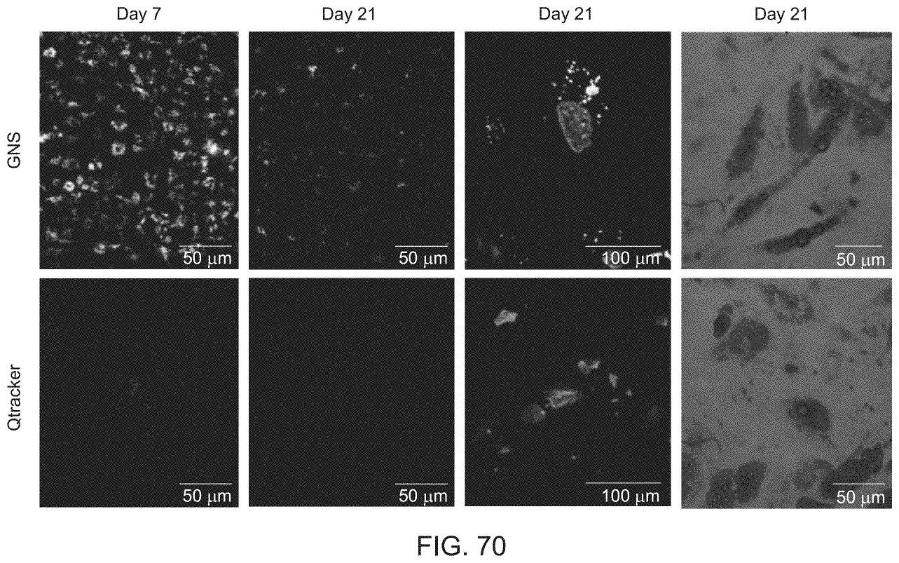

To evaluate whether AuNS-ASCs maintain the capacity for tri-lineage differentiation, AuNS-ASCs were differentiated for 21 days. Qtracker-labeled ASCs were differentiated to compare the TPL properties of both optical labels. After 21 days, cells were stained to validate their differentiated phenotype. Multiphoton mages were captured throughout differentiation. Imaging began at 7 days for adipogenesis (FIG. 55), osteogenesis, and chondrogenesis. FIG. 70 is a series of images showing gold nanostars as position sensors and trackers for adipose stem cells. For all studies, adipose-derived stem cells were incubated with 0.14 nM gold nanostars and 0.14 nM Qtracker for 24 hours before differentiation. (Left and second from left) For adipogenesis on days 7 and 21 of differentiation, images were taken with multiphoton microscopy to monitor the two-photon photoluminescence of the labels (scale bars=50 um). (Second from right) Images show labeled adipose-derived stem cells on day 21 of differentiation at 5× magnification with gold nanostars and Qtracker (scale bars=25 um). (Right) Images stained with Hoechst33342. This column represents the confirmation of phenotypic differentiation with Oil Red 0 for adipogenesis (scale bars=100 um). For adipogenesis, the cells labeled with gold nanostars could be easily visualized over a period of 21 days and to a greater extent than with the Qtracker label. Results similar to those shown in FIG. 70 were seen in osteogenesis and chondrogenesis as well. All samples were imaged with multiphoton microscopy using a wavelength of 800 nm at 3.7 mW.

AuNS-labeled cells exhibited strong TPL throughout differentiation, with only slight decay in signal over time. The decrease in signal intensity in these AuNS-labeled cells did not preclude the use of MPM. In comparison, Qtracker-labeled cells exhibited such rapid signal decay that by the 21st day, cells were not easily visualized. Analysis of AuNS and Qtracker-labeled cells using a two-way ANOVA demonstrated significantly greater TPL (p<0.001) for AuNS throughout tri-lineage differentiation. Lastly, the cytoplasmic localization of the AuNS remained undisrupted in all three lineages as demonstrated with Hoechst33342 staining. These studies show that AuNS effectively label ASCs without altering cell phenotype and exhibited stronger TPL than Qtracker throughout differentiation.

### Example 12. TAT-Peptide-Functionalized iMS Nanoprobes for Improved Intracellular Delivery

Cell-penetrating peptides can be used to overcome the lipophilic barrier of cellular membranes and deliver nanoprobe systems inside a cell for intracellular sensing. The TAT peptide (GRKKRRQRRRPQ), which is derived from the transactivator of transcription (TAT) of human immunodeficiency virus, can be used as a cell-penetrating peptide.

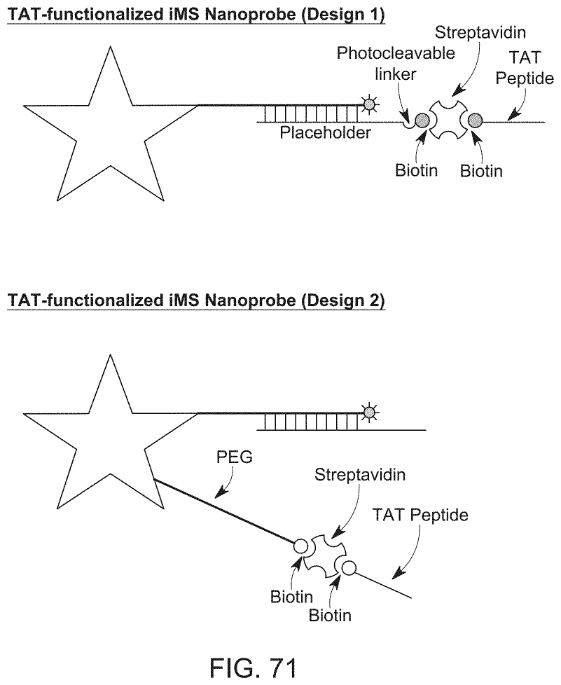

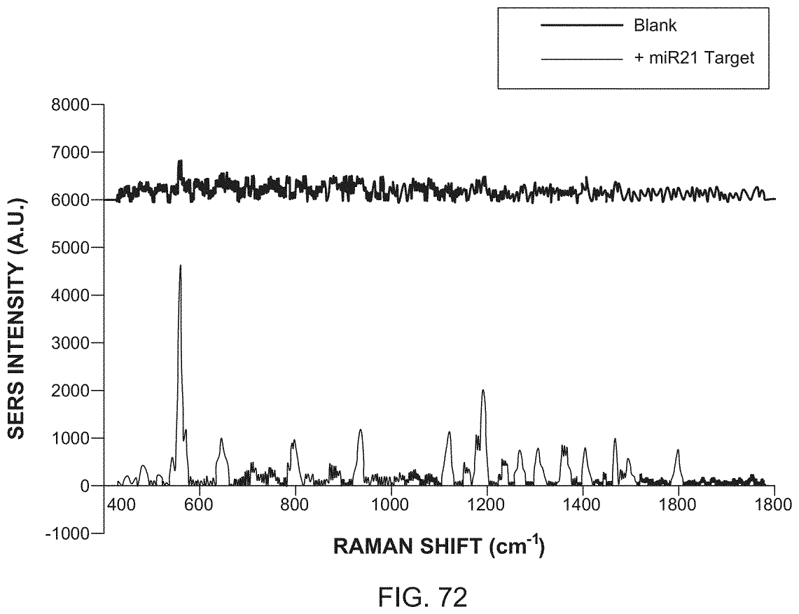

TAT peptide functionalized iMS nanoprobes can be used to improve cellular uptake and intracellular targeting. FIG. 71 is a schematic diagram of 2 embodiments of a TAT peptide funcationalized iMS nanoprobe. In embodiment 1, the TAT peptide is conjugated on a placeholder strand through the biotin-streptavidin linkage. A photocleavable linker can be added between the biotin and the placeholder for controllable release of the TAT-biotin-streptavidin complex inside cells. In embodiment 2, the TAT peptide is conjugated on a PEG polymer through the biotin-streptavidin linkage. FIG. 72 is a chart showing SERS spectra of the TAT-peptide-functionalized miR-21 iMS nanoprobe (embodiment 1) in the presence (+miR-21 Target) or absence (Blank) of miR-21 target sequences. The increased SERS intensity in the presence of targets indicates that the iMS nanoprobe keeps its functionality after conjugating with the TAT peptide.

### Example 13. In Vivo Diagnostics Using Photoactivated Nanoprobes

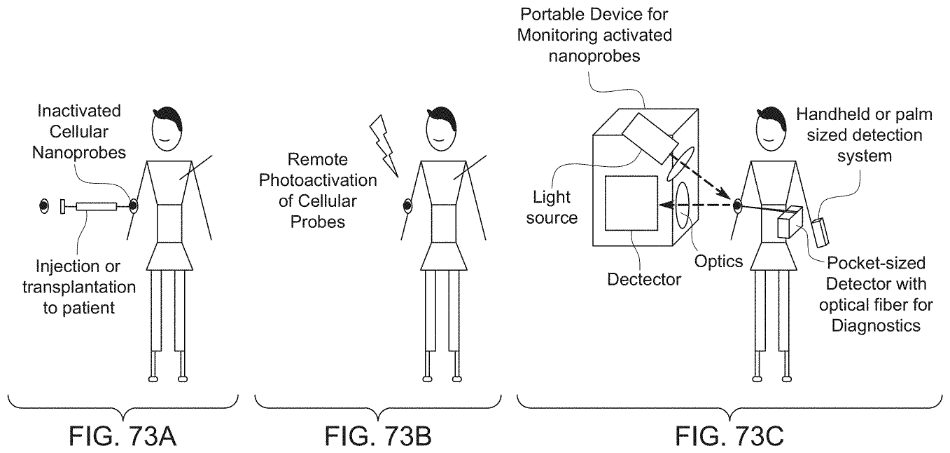

FIGS. 73A, 73B, and 73C are schematic diagrams showing an exemplary in vivo diagnostic modality for cellular systems having photo-activated nanoprobes. The cellular systems with inactivated nanoprobes can be transplanted to a subject. Following photoactivation using a photonics system with appropriate energy (light, microwave, radiowave energy, etc.), the activated nanoprobes are ready to serve as a real time and continuous monitor.

The nanoprobes can be monitored using various detection systems, including, for example: (1) a portable Raman diagnostic system having excitation light source and an optical detector, (2) a pocket-sized Raman diagnostics system with fiber optics excitation and detection; or (3) a handheld battery-operated Raman reader system can be operated remotely by an iPhone or similar device.

One skilled in the art will readily appreciate that the present disclosure is well adapted to carry out the objects and obtain the ends and advantages mentioned, as well as those inherent therein. The disclosure described herein as representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### REFERENCES

Quemener, A. M., Bachelot, L., Forestier, A., Donnou-Fournet, E., Gilot, D., & Galibert, M. D. (2020). The powerful world of antisense oligonucleotides: From bench to bedside. Wiley Interdisciplinary Reviews: RNA, e1594.
Stenvang, J., Petri, A., Lindow, M., Obad, S., & Kauppinen, S. (2012). Inhibition of microRNA function by anti-miR oligonucleotides. Silence, 3(1), 1.
Huynh, A., Madu, C. O., & Lu, Y. (2018) siRNA: A Promising New Tool for Future Breast Cancer Therapy. Oncomedicine, 3, 74-81.
Wang, H. N., Crawford, B. M., Fales, A. M., Bowie, M. L., Seewaldt, V. L., & Vo-Dinh, T. (2016). Multiplexed detection of microRNA biomarkers using SERS-based inverse molecular sentinel (iMS) nanoprobes. The Journal of Physical Chemistry C, 120(37), 21047-21055.
Si, M. L., Zhu, S., Wu, H., Lu, Z., Wu, F., & Mo, Y. Y. (2007). miR-21-mediated tumor growth. Oncogene, 26(19), 2799.
Yamankurt, G., Stawicki, R. J., Posadas, D. M., Nguyen, J. Q., Carthew, R. W., & Mirkin, C. A. (2020). The effector mechanism of siRNA spherical nucleic acids. Proceedings of the National Academy of Sciences.
Duffy, M. J., Synnott, N. C., & Crown, J. (2018). Mutant p53 in breast cancer: potential as a therapeutic target and biomarker. Breast cancer research and treatment, 170(2), 213-219.
Ubby, I., Krueger, C., Rosato, R., Qian, W., Chang, J., & Sabapathy, K. (2019). Cancer therapeutic targeting using mutant—p53-specific siRNAs. Oncogene, 38(18), 3415.
Vester, B., & Wengel, J. (2004). LNA (locked nucleic acid): high-affinity targeting of complementary RNA and DNA. Biochemistry, 43(42), 13233-13241.
Orum, H., & Wengel, J. (2001). Locked nucleic acids: a promising molecular family for gene-function analysis and antisense drug development. Current opinion in molecular therapeutics, 3(3), 239-243.
Nedaeinia, R., Sharifi, M., Avan, A., Kazemi, M., Rafiee, L., Ghayour-Mobarhan, M., & Salehi, R. (2016). Locked nucleic acid anti-miR-21 inhibits cell growth and invasive behaviors of a colorectal adenocarcinoma cell line: LNA-anti-miR as a novel approach. Cancer gene therapy, 23(8), 246.
Elmén, J., Thonberg, H., Ljungberg, K., Frieden, M., Westergaard, M., Xu, Y., . . . & Wahlestedt, C. (2005). Locked nucleic acid (LNA) mediated improvements in siRNA stability and functionality. Nucleic acids research, 33(1), 439-447.
Rosi, N. L., Giljohann, D. A., Thaxton, C. S., Lytton-Jean, A. K., Han, M. S., & Mirkin, C. A. (2006). Oligonucleotide-modified gold nanoparticles for intracellular gene regulation. Science, 312(5776), 1027-1030.
Dougan, J. A., Karlsson, C., Smith, W. E., & Graham, D. (2007) Enhanced oligonucleotide—nanoparticle conjugate stability using thioctic acid modified oligonucleotides. Nucleic acids research, 35(11), 3668-3675.
Lee, J. S., Lytton-Jean, A. K., Hurst, S. J., & Mirkin, C. A. (2007). Silver nanoparticle-oligonucleotide conjugates based on DNA with triple cyclic disulfide moieties. Nano letters, 7(7), 2112-2115.
Wu, Y. Y., & Kuo, H. C. (2020). Functional roles and networks of non-coding RNAs in the pathogenesis of neurodegenerative diseases. Journal of Biomedical Science, 27, 1-23.
Thome, A. D., Harms, A. S., Volpicelli-Daley, L. A., & Standaert, D. G. (2016). microRNA-155 regulates alpha-synuclein-induced inflammatory responses in models of Parkinson disease. Journal of Neuroscience, 36(8), 2383-2390.
Singh, A., & Sen, D. (2017). MicroRNAs in Parkinson's disease. Experimental brain research, 235(8), 2359-2374.
Moore, C., Kennedy, T., Antel, J., Bar-Or, A., & Dhaunchak, A. (2013). MicroRNA dysregulation in multiple sclerosis. Frontiers in genetics, 3, 311.
Henry, R. J., et al. (2019). Inhibition of miR-155 limits neuroinflammation and improves functional recovery after experimental traumatic brain injury in mice. Neurotherapeutics, 16(1), 216-230.
Almehmadi, K. A., Tsilioni, I., & Theoharides, T. C. (2020). Increased Expression of miR-155p5 in Amygdala of Children With Autism Spectrum Disorder. Autism Research, 13(1), 18-23.
Butovsky, O., et al. (2015). Targeting mi R-155 restores abnormal microglia and attenuates disease in SOD 1 mice. Annals of neurology, 77(1), 75-99.
Tian, J., An, X., & Niu, L. (2016). Role of microRNAs in cardiac development and disease. Experimental and therapeutic medicine, 13(1), 3-8.
Montgomery, R. L., et al. (2011). Therapeutic inhibition of miR-208a improves cardiac function and survival during heart failure. Circulation, 124(14), 1537-1547.
Hannus, M., Beitzinger, M., Engelmann, J C, Weickert, M. T., Spang, R., Hannus, S., & Meister, G. (2014). siPools: highly complex but accurately defined siRNA pools eliminate off-target effects. Nucleic acids research, 42(12), 8049-8061.
Lewis, H. D., Husain, A., Donnelly, R. J. et al. Creation of a novel peptide with enhanced nuclear localization in prostate and pancreatic cancer cell lines. BMC Biotechnol 10, 79 (28 Oct. 2010). https://doi.org/10.1186/1472-6750-10-79.
Byrnes C K, Nass P H, Duncan M D, Harmon J W. A nuclear targeting peptide, M9, improves transfection efficiency in fibroblasts. J Surg Res. 2002 November; 108(1):85-90. doi: 10.1006/jsre.2002.6517. PMID: 12443719.

That which is claimed:

1. A nanoprobe system for in vivo use, comprising
a plasmonic-active nanoparticle, and
a molecular probe system, comprising
an oligonucleotide capable of forming a stem-loop configuration, having a first end and a second end, wherein the oligonucleotide is immobilized to the plasmonic-active nanoparticle at the first end and labeled with a Raman reporter at the second end,
a placeholder strand at least partially bound to the oligonucleotide, and
an attachment mechanism for attachment to a cell membrane.

2. The nanoprobe system of claim 1, wherein the plasmonic-active nanoparticle comprises a metal.

3. The nanoprobe system of claim 1, wherein the plasmonic-active nanoparticle comprises a shell and a core.

4. The nanoprobe system of claim 1, wherein the plasmonic-active nanoparticle comprises a metal nanoparticle, a dielectric nanoparticle core covered with metal nanocap, a spherical metal nanoshell covering dielectric spheroid core, an oblate metal nanoshell covering dielectric spheroid core, a metal nanoparticle core covered with dielectric nanoshell, a metal nanoshell with protective coating layer, a multi-layer metal nanoshell covering a dielectric spheroid core, a multi-nanoparticle structure, a metal nanocube and nanotriangle/nanoprism, a metal cylinder, or a metal nanostar.

5. The nanoprobe system of claim 4, wherein the plasmonic-active nanoparticle is a metal nanoparticle and a surface of the metal nanoparticle is altered via fractionalization with PEG linkers having different functional groups.

6. The nanoprobe system of claim 1, wherein the plasmonic-active nanoparticle is a gold nanostar.

7. The nanoprobe system of claim 6, wherein the oligonucleotide comprises an internal spacer, and the internal spacer does not comprise adenine.

8. The nanoprobe system of claim 6, wherein the nanostar has a two-photon action cross section of up to 10⁶-10⁷ Goeppert-Mayer units (GM).

9. The nanoprobe system of claim 6, wherein the nanostar is functionalized with a TAT peptide.

10. The nanoprobe system of claim 1, wherein the nanoparticle is functionalized with folic acid.

11. The nanoprobe system of claim 1, wherein the nanoparticle is coated with silver.

12. The nanoprobe system of claim 11, wherein the silver-coated nanoparticle is coated with silica.

13. The nanoprobe system of claim 1, wherein the oligonucleotide is immobilized to the plasmonic-active nanoparticle with a metal-thiol bond.

14. The nanoprobe system of claim 1, wherein the oligonucleotide comprises a stem-L, a stem-R, a spacer and a placeholder segment.

15. The nanoprobe system of claim 1, wherein the oligonucleotide comprises one or more of DNA bases, 2′-O-methyl purines, 2′-fluoropyrimidines, PS linkage modifications, and terminal inverted-dT bases.

16. The nanoprobe system of claim 1, wherein the oligonucleotide comprises a phosphorothioate bond modification.

17. The nanoprobe system of claim 1, wherein the oligonucleotide comprises a 2′-O-methylation (2′OMe) modification.

18. The nanoprobe system of claim 1, wherein the oligonucleotide comprises a locked nucleic acid (LNA) modification.

19. The nanoprobe system of claim 18, wherein the oligonucleotide further comprises a 5 base-pair stem and alternating DNA/LNA bases.

20. The nanoprobe system of claim 1, wherein the attachment mechanism comprises a membrane anchor, an antibody, a peptide, or ligand.

21. The nanoprobe system of claim 20, wherein the membrane anchor is selected from the group consisting of lipids, cholesterol, porphyrin, tocopherol, acyl chain, oleyl chain, a monothiol or multi-thiol anchoring group, and dioleylphosphatidylethanolamine.

22. The nanoprobe system of claim 1, wherein the system is encapsulated in a gel matrix.

23. The nanoprobe system of claim 22, wherein the gel matrix comprises a poly-NIPAM gel matrix.

24. The nanoprobe system of claim 1, comprising at least two plasmonic-active nanoparticles, each nanoparticle having an oligonucleotide with a Raman reporter immobilized thereto, a placeholder strand and an attachment mechanism, wherein the at least two Raman reporters are different.

25. The nanoprobe system of claim 24, wherein the Raman reporters are selected from the group consisting of Cy5, Cy5.5, TYE665, TYE563, Rhodamine Red, TAMRA, and Cy3, and wherein different Raman reporters are used with different oligonucleotides.

26. The nanoprobe system of claim 1, wherein the placeholder strand comprises an aptamer.

27. The nanoprobe system of claim 1, wherein the placeholder strand acts as an antisense oligonucleotide and is linked to a small interfering RNA (siRNA).

28. The nanoprobe system of claim 27, wherein the placeholder strand targets one or more of DNA or RNA in a nucleus or cytoplasm, such as genomic DNA, pre-mRNA, mRNA, microRNA (miRNA) as well as various non-coding RNAs (ncRNAs)

29. The nanoprobe system of claim 28, wherein the placeholder strand is an anti-miR-21.

30. The nanoprobe system of claim 28, wherein the siRNA targets R175H p53 mutant.

31. The nanoprobe system of claim 27, wherein the siRNA is single stranded siRNA or double stranded siRNA.

32. The nanoprobe system of claim 27, wherein the siRNA is linked to the placeholder strand with a spacer, a linker, or both a spacer and a linker

33. A method of in vivo monitoring and/or detection comprising
administering a nanoprobe system to a subject, the nanoprobe system comprising a plasmonic-active nanoparticle; an oligonucleotide having a first end and a second end, wherein the oligonucleotide is immobilized to the plasmonic-active nanoparticle at the first end and labeled with a Raman reporter at the second end; a placeholder strand complimentary to and at least partially bound to the oligonucleotide; and an attachment mechanism for attachment of the nanoprobe system to a cell membrane,
wherein the placeholder strand targets a specific sequence and upon exposure of the nanoprobe system to the specific target sequence, the placeholder strand leaves the oligonucleotide in favor of the target sequence, allowing the oligonucleotide to fold into a closed stem-loop configuration whereby the Raman reporter is near or on the plasmonic-active nanoparticle surface thereby yielding a SERS signal, and
detecting the SERS signal with a detection device.

34. The method of claim 33, further comprising ongoing detecting and monitoring of the SERS signal for a time period.

35. The method of claim 33, wherein the detection device is portable.

36. The method of claim 33, wherein the detection device is pocket-sized, hand-sized, or wristwatch sized.

37. The method of claim 33, wherein the specific target sequence comprises one or more of DNA or RNA in a nucleus or cytoplasm, such as genomic DNA, pre-mRNA, mRNA, microRNA (miRNA) as well as various non-coding RNAs (ncRNAs).

38. The method of claim 33, wherein the specific target sequence is related to one or more of brain disease, cancer, neuroinflammatory and neurodegenerative disorders, and cardiac disorders or diseases.

39. The method of claim 33, wherein the specific target sequence is related to one or more of Parkinson's disease, Alzheimer's disease, multiple sclerosis, amyotrophic lateral sclerosis (ALS), traumatic brain injury, and autism.

40. The method of claim 33, wherein the specific target sequence includes one or more of miR-21, miR-155, and miR-208a.

41. The method of claim 33, wherein administration comprises intravenous injection, intra-arterial infusion, intrathecal infusion, or subcutaneous implantation.

42. The method of claim 33, wherein the nanoprobe system is used for real-time, continuous detection of a specific target sequence.

43. The method of claim 42, wherein the nanoprobe system is used to monitor stem cells or tissue engineering organs.

44. The method of claim 33, wherein the method comprises using two-photon photoluminescence (TPL) microscopy.

45. The method of claim 33, wherein the nanoprobe system is administered to the subject in an inactive state, and the method further comprises externally activating the nanosensor at a time after administration.

46. The method of claim 45, wherein the nanoprobe system is externally photoactivatable via a photocleavable linker in the oligonucleotide.

47. The method of claim 33, wherein the nanoprobe system comprises a plurality of differently labeled nanoprobe systems thus enabling sensing of multiple targets or biological macromolecules simultaneously in a single assay platform.

48. A method of in vivo therapy comprising
administering a nanoprobe system to a subject in need of a desired therapy, the nanoprobe system comprising a plasmonic-active nanoparticle; an oligonucleotide having a first end and a second end, wherein the oligonucleotide is immobilized to the plasmonic-active nanoparticle at the first end and labeled with a Raman reporter at the second end; a placeholder strand complimentary to and at least partially bound to the oligonucleotide; a component for inducing molecular regulation linked to the placeholder strand; and an attachment mechanism for attachment of the nanoprobe system to a cell membrane,
wherein the placeholder strand targets a specific sequence, and upon exposure of the nanoprobe system to the specific target sequence, the placeholder strand leaves the oligonucleotide in favor of the target sequence, allowing the oligonucleotide to fold into a closed stem-loop configuration whereby the Raman reporter is near or on the plasmonic-active nanoparticle surface thereby yielding a SERS signal, and wherein the component for inducing molecular regulation separates from the placeholder strand thereby initiating the desired therapy, and detecting the SERS signal with a detection device.

49. The method of claim 48, wherein the molecular regulation mechanism induced by the component for inducing molecular regulation comprises targeted gene silencing, antisense-based blocking, mRNA blocking, miRNA blocking, or aptamer-based molecular regulation.

50. The method of claim 48, wherein the nanoprobe system is administered in an inactive state, and the method further comprises externally activating the nanoprobe system at a time after administration.

51. The method of claim 50, wherein the nanoprobe system is externally photoactivatable.

52. The method of claim 50, wherein administration comprises injection, infusion, implantation or transplantation.

53. The method of claim 48, wherein the specific target sequence comprises one or more of DNA or RNA in a nucleus or cytoplasm, such as genomic DNA, pre-mRNA, mRNA, microRNA (miRNA) as well as various non-coding RNAs (ncRNAs).

54. The method of claim 48, further comprising ongoing detecting and monitoring of the SERS signal for a time period.

55. The method of claim 48, wherein the specific target sequence is related to one or more of brain disease, cancer, neuroinflammatory and neurodegenerative disorders, and cardiac disorders or diseases.

56. The method of claim 48, wherein the specific target sequence is related to one or more of Parkinson's disease, Alzheimer's disease, multiple sclerosis, amyotrophic lateral sclerosis (ALS), traumatic brain injury, and autism.

57. The method of claim 48, wherein the detection device is portable.

58. The method of claim 48, wherein the detection device is pocket-sized, hand-sized, or wristwatch sized.
